(19) United States
(12) Patent Application Publication
(10) Pub. No.: US 2021/0290514 A1
(43) Pub. Date: Sep. 23, 2021

(54) An Antimicrobial Combination Composition Comprising Glycerol Derivatives and Bicyclic Compounds

(71) Applicants: CLARIANT INTERNATIONAL LIMITED, Muttenz 1 (CH); SACHEM EUROPE B.V., Zalbommel (NL)

(72) Inventors: Tom Fricke, Liederbach (DE); Tim Engel, Tilburg (NL); Paul Janssen, Nijmegen (NL)

(21) Appl. No.: 17/257,718

(22) PCT Filed: Jun. 7, 2019
(86) PCT No.: PCT/EP2019/064952

§ 371 Date: Jan. 4, 2021

Related U.S. Application Data
(60) Provisional application No. 62/694,442, filed on Jul. 5, 2018.

(51) Int. Cl. A61K 8/60; A61K 8/34; A61K 8/49; A61Q 19/00

(52) U.S. Cl. CPC A61K 8/602; A61K 8/345; A61Q 17/005; A61Q 19/00; A61K 8/498

(57) ABSTRACT

The present disclosure relates to inter alia an antimicrobial combination composition comprising: (a) one or more glycerol derivatives; and (b) one or more bicyclic compounds. The antimicrobial combination composition can be used in cosmetic formulations or pharmaceutical formulations.

### FIELD OF THE INVENTION

The present disclosure relates to inter alia an antimicrobial combination composition comprising: (a) one or more glycerol derivatives; and (b) one or more bicyclic compounds. The antimicrobial combination composition can be used in cosmetic formulations or pharmaceutical formulations.

### BACKGROUND OF THE INVENTION

Preservation of household formulations, such as cosmetic formulations, extends their shelf life and therefore provides greater value for money for consumers. Furthermore, preservatives prevent consumers from distributing microbes around their home or on themselves and hence provide health benefits. Anti-microbial actives are well-described in the art and there are many available that provide excellent performance.

Pesaro et al in WO2014191258A2 (Symrise) relates to Antimicrobial Compositions Comprising Glyceryl Ethers. Pesaro et al discloses octylglycerin in paragraphs 0024 and 00234.

### SUMMARY OF THE INVENTION

In a first aspect, the present invention relates inter alia to an antimicrobial combination composition including:
(a) one or more glycerol derivatives selected from the group consisting of: 3-[(2-ethylhexyl)oxy]-1,2-propanediol, 3-[(n-octyl)oxy]-1,2-propanediol, and 3-[(2-octyl)oxy]-1,2-propanediol; and
(b) one or more bicyclic compounds selected from the group consisting of: 5,7-dihydroxy-2-(4-hydroxyphenyl)chroman-4-one (naringenin), 2-(3,4-dihydroxyphenyl)-3,5,7-trihydroxychromen-4-one (quercetin), 5,7-dihydroxy-2-(3-hydroxy-4-methoxyphenyl)chroman-4-one (hesperetin), and 5,7-dihydroxy-2-(3,4-dihydroxyphenyl)-chroman-4-one (eriodyctiol).

A second aspect relates to the antimicrobial combination composition of the first aspect for use as a medicament. Another embodiment of the second aspect relates to the antimicrobial agent, of the first aspect, for use in (prophylactically) treating infections on the human or animal body.

A third aspect relates to a cosmetic composition comprising the antimicrobial combination composition according to first aspect; specifically the third aspect relates to a cosmetic composition including:
(a) one or more glycerol derivatives selected from the group consisting of: 3-[(2-ethylhexyl)oxy]-1,2-propanediol, 3-[(n-octyl)oxy]-1,2-propanediol, and 3-[(2-octyl)oxy]-1,2-propanediol; and
(b) one or more bicyclic compounds selected from the group consisting of: 5,7-dihydroxy-2-(4-hydroxyphenyl)chroman-4-one (naringenin), 2-(3,4-dihydroxyphenyl)-3,5,7-trihydroxychromen-4-one (quercetin), 5,7-dihydroxy-2-(3-hydroxy-4-methoxyphenyl)chroman-4-one (hesperetin), and 5,7-dihydroxy-2-(3,4-dihydroxyphenyl)-chroman-4-one (eriodyctiol).

In certain embodiments, the cosmetic composition is for leave-on skin care use having a pH in the range of 4-6.

A fourth aspect relates to a method of caring for keratinous material, comprising:
(1) Applying the cosmetic composition according to the third aspect or the antimicrobial composition according to the first aspect onto keratinous material; and
(2) Allowing the composition to remain on the keratinous material for at least 1 hour.

A fifth aspect relates to the use of the antimicrobial combination composition of the first aspect, in preserving cosmetic compositions.

### DETAILED DESCRIPTION OF THE INVENTION

### Definitions and General

In this document, including in all embodiments of all aspects of the present invention, the following definitions apply unless specifically stated otherwise. All percentages are by weight (w/w) of the total composition. All ratios are weight ratios. “wt %” means percentage by weight. References to ‘parts’ e.g. a mixture of 1 part X and 3 parts Y, is a ratio by weight. “QS” or “QSP” means sufficient quantity for 100% or for 100 g. +/− indicates the standard deviation. All ranges are inclusive and combinable. The number of significant digits conveys neither a limitation on the indicated amounts nor on the accuracy of the measurements. All numerical amounts are understood to be modified by the word “about”. All measurements are understood to be made at 23° C. and at ambient conditions, where “ambient conditions” means at 1 atmosphere (atm) of pressure and at 50% relative humidity. “Relative humidity” refers to the ratio (stated as a percent) of the moisture content of air compared to the saturated moisture level at the same temperature and pressure. Relative humidity can be measured with a hygrometer, in particular with a probe hygrometer from VWRO International. Herein “min” means “minute” or “minutes”. Herein “mol” means mole. Herein “g” following a number means “gram” or “grams”. “Ex.” means “example”. All amounts as they pertain to listed ingredients are based on the active level (‘solids’) and do not include carriers or by-products that may be included in commercially available materials. Herein, “comprising” means that other steps and other ingredients can be in addition. “Comprising” encompasses the terms “consisting of” and “consisting essentially of”. The compositions, formulations, methods, uses, kits, and processes of the present invention can comprise, consist of, and consist essentially of the elements and limitations of the invention described herein, as well as any of the additional or optional ingredients, components, steps, or limitations described herein. Embodiments and aspects described herein may comprise or be combinable with elements, features or components of other embodiments and/or aspects despite not being expressly exemplified in combination, unless an incompatibility is stated. “In certain embodiments” means that one or more embodiments, optionally all embodiments or a large subset of embodiments, of the present invention has/have the subsequently described feature. Where amount ranges are given, these are to be understood as being the total amount of said ingredient in the composition, or where more than one species fall within the scope of the ingredient definition, the total amount of all ingredients fitting that definition, in the composition. For example, if the composition comprises from 1% to 5% fatty alcohol, then a composition comprising 2% stearyl alcohol and 1% cetyl alcohol and no other fatty alcohol, would fall within this scope.

“Independently selected from,” means that the referenced groups can be the same, different, or a mixture thereof, unless the context clearly indicates otherwise. Thus, under this definition, the phrase “X1, X2, and X3 are independently selected from noble gases” would include the scenario where X1, X2, and X3 are all the same, where X1, X2, and X3 are all different, and where X1 and X2 are the same but X3 is different.

“Molecular weight” or “M.Wt.” or “MW” and grammatical equivalents mean the number average molecular weight.

“Viscosity” is measured at 25° C. using a HAAKE Rotation Viscometer VT 550 with cooling/heating vessel and sensor systems according to DIN 53019 at a shear rate of 12.9 s⁻¹.

“Water-soluble” refers to any material that is sufficiently soluble in water to form a clear solution to the naked eye at a concentration of 0.1% by weight of the material in water at 25° C. The term “water-insoluble” refers to any material that is not “water-soluble”.

“Dry” or “substantially dry” means comprising less than 5%, less than 3% or, less than 2%, less than 1%, or about 0% of any compound or composition being in liquid form when measured at 25° C. at ambient conditions. Such compounds or compositions being in liquid form include water, oils, organic solvents and other wetting agents. “Anhydrous” means that the composition comprises less than 5%, less than 3% or, less than 2%, less than 1%, or about 0% water by total weight of the composition.

“Substantially free from” or “substantially free of” means less than 1%, or less than 0.8%, or less than 0.5%, or less than 0.3%, or about 0%, by total weight of the composition or formulation.

“Hair” means mammalian keratin fibres including scalp hair, facial hair and body hair. It includes such hair still being attached to a living subject and also hair that has been removed therefrom such as hair swatches and hair on a doll/mannequin. In certain embodiments, “hair” means human hair. “Hair shaft” or “hair fibre” means an individual hair strand and may be used interchangeably with the term “hair.”

“Proximal to the scalp” means that portion of an extended, or substantially straightened, hair shaft that is closer in distance to the scalp than to the end of the hair. Thus, about 50% of the hair fibre length would be considered proximal to the scalp, and about 50% of the hair fibre would be distal to the scalp. “z cm proximal to the scalp” means a distance “z” along the hair, with one endpoint being on or directly adjacent to the scalp, and the second endpoint being measured “z” centimetres along the length of the extended or substantially straightened hair.

“Chemically modify” or grammatical equivalents thereof, means that a chemical moiety such as monomer and/or crosslinker and/or polymer, stably affixes to a second chemical moiety, for example, a keratin protein, another component of hair, and/or another monomer or crosslinker or polymer. Normally, “chemically modify” means stably affix via a covalent bond, unless otherwise stated. “Cosmetically acceptable” means that the compositions, formulations or components described are suitable for use in contact with human keratinous tissue without undue toxicity, incompatibility, instability, allergic response, and the like. All compositions and formulations described herein which have the purpose of being directly applied to keratinous tissue are limited to those being cosmetically acceptable.

“Derivatives” includes but is not limited to, amide, ether, ester, amino, carboxyl, acetyl, acid, salt and/or alcohol derivatives of a given compound. In certain embodiments, “derivatives thereof” means the amide, ether, ester, amino, carboxyl, acetyl, acid, salt and alcohol derivatives.

“Monomer” means a discrete, non-polymerised chemical moiety capable of undergoing polymerisation in the presence of an initiator or any suitable reaction that creates a macromolecule e.g. such as polycondensation, polyaddition, anionic or cationic polymerization. “Unit” means a monomer that has already been polymerised i.e. is part of a polymer.

“Polymer” means a chemical formed from the polymerisation of two or more monomers. The term “polymer” shall include all materials made by the polymerisation of monomers as well as natural polymers. Polymers made from only one type of monomer are called homopolymers. Herein, a polymer comprises at least two monomers. Polymers made from two or more different types of monomers are called copolymers. The distribution of the different monomers can be random, alternating or block-wise (i.e. block copolymer). The term “polymer” used herein includes any type of polymer including homopolymers and copolymers.

“Kit” means a package comprising a plurality of components. “Kit” may be referred to as “kit-of-parts”. An example of a kit is, for example, a first composition and a separately packaged second composition and optionally application instructions.

The details of the invention and its aspects are provided hereinafter.

### First Aspect

The first aspect relates to an antimicrobial combination. As descried above, the antimicrobial combination composition includes (a) one or more glycerol derivatives, and (b) one or more bicyclic compounds.

As described above, the antimicrobial composition includes one or more glycerol derivatives. In certain embodiments of the disclosure, the glycerol derivative is selected from the group consisting of: 3-[(2-ethylhexyl)oxy]-1,2-propanediol, 3-[(n-octyl)oxy]-1,2-propanediol, and 3-[(2-octyl)oxy]-1,2-propanediol. In certain embodiments, the glycerol derivative is selected from the group consisting of: 3-[(n-octyl)oxy]-1,2-propanediol, and 3-[(2-octyl)oxy]-1,2-propanediol. In certain embodiments, the one or more glycerol derivatives is 3-[(n-octyl)oxy]-1,2-propanediol and 3-[(2-octyl)oxy]-1,2-propanediol. In certain embodiments, the one or more glycerol derivatives is 3-[(n-octyl)oxy]-1,2-propanediol. In certain embodiments, the one or more glycerol derivatives is 3-[(2-octyl)oxy]-1,2-propanediol. In certain embodiments, the one or more glycerol derivatives is 3-[(2-ethylhexyl)oxy]-1,2-propanediol.

In certain embodiments, the glycerol derivative may be selected from the compounds of formula (I) provided in International Patent Publication WO 2018/125734, incorporated by reference herein in its entirety.

In certain embodiments, the antimicrobial combination composition is a concentrate (i.e., in a concentration suitable for further dilution prior to end use). In certain embodiments, the antimicrobial combination composition is a working mixture (i.e., in a concentration suitable for end use).

The glycerol derivative of the disclosure is preferably provided at a purity of at least 92%, prior to its introduction into the antimicrobial combination composition. In certain embodiments, the glycerol derivative of the disclosure is provided at a purity of at least 99.99%, or at least 99.90%, or at least 99%, or at least 98%, or at least 94%, prior to its introduction into the antimicrobial combination composition. In certain embodiments, the glycerol derivative of the disclosure is provided at a purity of at least 99.99%, prior to its introduction into the antimicrobial combination composition. These levels of purity of the one or more glycerol derivatives ensure that the composition contains few, if any, impurities that might be ingested or absorbed (e.g., through the skin) by a subject using the antimicrobial combination composition of the disclosure, either as a concentrate (or the resultant a cosmetic or pharmaceutical formulation comprising the antimicrobial combination composition of the disclosure) or as a working mixture.

As described above, the antimicrobial combination composition includes one or more bicyclic compounds. In certain embodiments of the disclosure, the bicyclic compound is selected from the group consisting of: 5,7-dihydroxy-2-(4-hydroxyphenyl)chroman-4-one (naringenin), 2-(3,4-dihydroxyphenyl)-3,5,7-trihydroxychromen-4-one (quercetin), 5,7-dihydroxy-2-(3-hydroxy-4-methoxyphenyl)chroman-4-one (hesperetin), and 5,7-dihydroxy-2-(3,4-dihydroxyphenyl)-chroman-4-one (eriodyctiol). In certain embodiments, the one of more bicyclic compounds is naringenin. In certain embodiments, the one of more bicyclic compounds is quercetin. In certain embodiments, the one of more bicyclic compounds is hesperetin. In certain embodiments, the one of more bicyclic compounds is eriodyctiol.

In certain embodiments, the bicyclic compound may be selected from the compounds of formula (II) provided in International Patent Publication WO 2018/125734, incorporated by reference herein in its entirety.

The bicyclic compound of the disclosure can be, for example, provided at a purity of at least 85%, prior to its introduction into the antimicrobial combination composition. In certain embodiments, the bicyclic compound of the disclosure is provided at a purity of at least 99.99%, or at least 99.90%, or at least 99%, or at least 98%, or at least 94%, or at least 92%, or at least 90%, or at least 89%, or at least 87%, prior to its introduction into the antimicrobial combination composition. In certain embodiments, the bicyclic compound of the disclosure is provided at a purity of at least 99.99%, prior to its introduction into the antimicrobial combination composition. As described above with respect to the glycerol derivatives, these levels of purity of the bicyclic compound ensure consumer safe antimicrobial combination composition of the disclosure.

In certain embodiment, the one or more bicyclic compounds is present in an amount in the range of about 50 ppm to about 50000 ppm (equivalent to 0.005 wt % to 5 wt %), based on the total weight of the one or more glycerol derivatives. Thus, using this embodiment as an example, to a antimicrobial combination composition (e.g., am antimicrobial concentrate) containing otherwise substantially pure glycerol derivative, there will be added from about 50 ppm to about 50000 ppm of the one or more bicyclic compounds. In certain embodiments, one or more bicyclic compounds is present in an amount in the range of about 100 ppm to about 10000 ppm, or about 200 ppm to about 5000 ppm, or about 500 ppm to about 1000 ppm, or about 300 ppm to about 900 ppm, based on the total weight of the one or more glycerol derivatives.

In certain embodiment, the one or more bicyclic compounds is present in an amount less than 5 wt. %, or less than 3 wt. %, or less than 1 wt. %, or even less than 0.5 wt. %. In certain embodiments, the one or more bicyclic compounds is naringenin (5,7-dihydroxy-2-(4-hydroxyphenyl)chroman-4-one), and is present in an amount less than 5 wt. %, or less than 3 wt. %, or less than 1 wt. %, or even less than 0.5 wt. %, based on the total weight of the one or more glycerol derivatives.

When an antimicrobial combination composition of the disclosure is provided as a concentrate further used in a cosmetic formulation or a pharmaceutical formulation, the antimicrobial combination composition may be further diluted (e.g., by adding it to another solution) yet the amount of the one or more bicyclic compounds as described above will remain relative to the amount of the one or more glycerol derivatives. Optionally, it may be desirable to add additional amount of the one or more bicyclic compounds to the cosmetic formulation or the pharmaceutical formulation, as additional antioxidant for the cosmetic or pharmaceutical formulation.

In one embodiment, the antimicrobial combination composition is a concentrate containing from 0.01 wt % to 5 wt % of the one or more bicyclic compounds, based on the total weight of the one or more glycerol derivatives. In one embodiment, the antimicrobial combination composition is a concentrate containing any above-disclosed amount of the one or more bicyclic compounds, based on the total weight of the one or more glycerol derivatives. In another embodiment, the concentrate includes one or more other ingredients as described below.

In certain embodiments, the antimicrobial combination composition of the disclosure is in a form of a working mixture (i.e., in a concentration suitable for end use). A working mixture may include up to about 60% by weight (e.g., from 10% by weight to about 60% by weight) of the antimicrobial combination composition as defined herein. To obtain such a working mixture, the antimicrobial combination composition as described herein can be dissolved in (i.e., diluted by) a suitable amount of an additive (e.g., a diluent or an excipient). For example, in certain embodiments, the antimicrobial combination composition may be diluted in water, alcohol(s), polyol(s), or mixtures thereof to obtain a working mixture. In such working mixtures, the amount of the one or more bicyclic compounds as described above will remain relative to the amount of one or more glycerol derivatives even after the dilution of the antimicrobial combination composition. Optionally, it may be desirable to add additional amount of the one or more bicyclic compounds to the working mixture.

The antimicrobial combination composition of the disclosure as described herein, either in the form of a concentrate or as a working mixture, can be added to cosmetic and/or pharmaceutical formulation, for example, as is known from the use of glycerol derivatives, such as glycerol monoalkyl ethers. In other embodiments, the antimicrobial combination compositions as described herein may be used in technical products which are intended to be provided with glycerol derivatives and in which peroxides are undesired, e.g., preparations comprising compounds which contain dyes or perfumes or which are unsaturated or sensitive to oxidation. Such formulations or technical products may include, for example, deodorants, skincare products, sunscreens, baby products, cosmetics, aftershaves, disinfectants, antiseptics, washing lotions, in-shower lotions, and hair treatment compositions (e.g., leave-in or rinse out compositions). Thus, in certain embodiments, the antimicrobial combination compositions is completely free or substantially free one or more peroxides (e.g., less than 1000 ppm, or less than 100 ppm, or less than 10 ppm, or even less than 1 ppm of peroxide(s) are present in the antimicrobial combination composition of the disclosure as otherwise described herein).

In an example embodiment of the First Aspect, the disclosure provides the antimicrobial combination compositions as described herein, whether added as concentrates or working mixtures, are used in the preparations such as those examples above, such that the corresponding cosmetic formulation, pharmaceutical formulation, or technical preparation contains from about 0.05 to about 5 wt %, or in another embodiment, from about 0.1 to about 1 wt %, or in another embodiment, from about 0.2 to about 0.6 wt %, or, in other embodiments 0.3 wt % or 0.5%, of the one or more glycerol derivatives as defined herein. It is noted that, as the purity of the compound of the one or more glycerol derivatives increased, e.g., from 94% pure up to 98% pure, prior to distillation, a somewhat higher amount of the one or more bicyclic compounds may be required. Without being bound by a theory, it is believe that a higher purity glycerol derivative may need higher stabilization.

### Second Aspect

A second aspect relates to the antimicrobial combination composition as otherwise described herein (e.g., of the first aspect) for use as a medicament. Another embodiment of the second aspect relates to the antimicrobial combination composition as otherwise described herein (e.g., of the first aspect) for use in (prophylactically) treating infections on the human or animal body.

### Third Aspect

A third aspect relates to a cosmetic composition comprising the antimicrobial combination composition as otherwise described herein (e.g., according to the first aspect). Specifically, the third aspect relates to a cosmetic composition comprising:
(a) one or more glycerol derivatives selected from the group consisting of: 3-[(2-ethylhexyl)oxy]-1,2-propanediol, 3-[(n-octyl)oxy]-1,2-propanediol, and 3-[(2-octyl)oxy]-1,2-propanediol; and
(b) one or more bicyclic compounds selected from the group consisting of: 5,7-dihydroxy-2-(4-hydroxyphenyl)chroman-4-one (naringenin), 2-(3,4-dihydroxyphenyl)-3,5,7-trihydroxychromen-4-one (quercetin), 5,7-dihydroxy-2-(3-hydroxy-4-methoxyphenyl)chroman-4-one (hesperetin), and 5,7-dihydroxy-2-(3,4-dihydroxyphenyl)-chroman-4-one (eriodyctiol).

The cosmetic compositions of the disclosure as described herein can advantageously be used even when having low pH values. For example, in certain embodiments, the cosmetic composition has a pH value in the range of 2.0 to 7.0, or 3.0 to 7.0, or 4.5 to 7.0, or pH 4 to pH 6.5, or pH 4 to pH 6, or pH 4.5 to pH 6.5, or pH 4.5 to pH 6, or pH 5.3 to pH 5.7, or pH 5 to pH 6, or pH of about 5.5. By varying the pH value, the cosmetic composition can be made available that is suitable for different applications.

In certain embodiments, the cosmetic composition as otherwise described herein is for leave-on skin care use having a pH in the range of 4-6.

As noted above, the cosmetic composition of the disclosure as otherwise described herein, in certain embodiments, includes the antimicrobial combination composition as otherwise described herein (e.g., according to the first aspect) in an amount of up to about 60% by weight, based on the total weight of the cosmetic composition. For example, in certain embodiments, the cosmetic composition of the disclosure includes from 1% by weight to about 60% by weight of the antimicrobial combination composition, or in the range of 10 wt % to 60 wt %, or 20 wt % to 60 wt %, or 30 wt % to 60 wt %, or 40 wt % to 60 wt %, or 50 wt % to 60 wt %, or 1 wt % to 40 wt %, or 10 wt % to 40 wt %, or 20 wt % to 40 wt %, or 30 wt % to 40 wt %, or 1 wt % to 30 wt %, or 10 wt % to 30 wt %, or 20 wt % to 30 wt %, or 1 wt % to 20 wt %, or 10 wt % to 20 wt %, or 15 wt % to 20 wt %, or 1 wt % to 15 wt %, or 1 wt % to 10 wt %, or 1 wt % to 5 wt % of the antimicrobial combination composition, based on the total weight of the cosmetic composition.

The amount of the one or more glycerol derivatives present in the cosmetic composition is present in an amount in the range of about 0.0005 wt % to about 5 wt %. For example, in certain embodiments, the one or more glycerol derivatives is present in the range of about 0.001 wt % to about 5 wt %, or from about 0.01 wt % to about 5 wt %, or from about 0.1 wt % to about 5 wt %, or from about 1 wt % to about 5 wt %, or 0.0005 wt % to about 1 wt %, or from about 0.001 wt % to about 1 wt %, or from about 0.01 wt % to about 1 wt %, or from about 0.1 wt % to about 1 wt %, or from about 0.5 wt % to about 1 wt %, or 0.0005 wt % to about 0.5 wt %, or from about 0.001 wt % to about 0.5 wt %, or from about 0.01 wt % to about 0.5 wt %, or from about 0.1 wt % to about 0.5 wt %, or from about 0.5 wt % to about 0.5 wt %, or 0.0005 wt % to about 0.1 wt %, or from about 0.001 wt % to about 0.1 wt %, or from about 0.01 wt % to about 0.1 wt %, about 0.05 wt % to about 5 wt %, or from about 0.1 wt % to about 1 wt %, or from about 0.1 wt % to about 0.6 wt %, or from about 0.1 wt % to about 0.5 wt %, or from about 0.1 wt % to about 0.4 wt %, or from about 0.2 wt % to about 1 wt %, or from about 0.2 wt % to about 0.6 wt %, or from about 0.2 wt % to about 0.5 wt %, or from about 0.2 wt % to about 0.4 wt %, or from about 0.4 wt % to about 1 wt %, or from about 0.4 wt % to about 0.6 wt %, or from about 0.4 wt % to about 0.5 wt %, or about 0.3 wt %, or about 0.5%, based on the total weight of the cosmetic composition.

The amount of the one or more bicyclic compounds in the cosmetic composition is present in an amount in the range of about 50 ppm to about 50000 ppm (equivalent to 0.005 wt % to 5 wt %), based on the total weight of the one or more glycerol derivatives present in the cosmetic composition. In certain embodiments, one or more bicyclic compounds is present in an amount in the range of about 100 ppm to about 10000 ppm, or about 200 ppm to about 5000 ppm, or about 500 ppm to about 1000 ppm, or about 300 ppm to about 900 ppm, based on the total weight of the one or more glycerol derivatives present in the cosmetic composition. In certain embodiments, the cosmetic composition of the disclosure may include an additional amount of the one or more bicyclic compounds (i.e., in addition to those provided with respect to the antimicrobial combination composition).

In certain embodiment, the one or more bicyclic compounds is present in an amount less than 5 wt. %, or less than 3 wt. %, or less than 1 wt. %, or even less than 0.5 wt. %. In certain embodiments, the one or more bicyclic compounds is naringenin (5,7-dihydroxy-2-(4-hydroxyphenyl)chroman-4-one), and is present in an amount less than 5 wt. %, or less than 3 wt. %, or less than 1 wt. %, or even less than 0.5 wt. %, based on the total weight of the one or more glycerol derivatives.

In certain embodiments, the cosmetic compositions of the disclosure as described herein may further comprise one or more additives selected from surfactants and auxiliary agents. Suitable surfactants and auxiliary agents are provided in more detail below. In certain embodiments, the one or more additives is acceptable for cosmetically use (i.e., they are cosmetically acceptable).

### Surfactant

In certain embodiments, the cosmetic composition of the disclosure as otherwise described herein comprises one or more surfactants. In certain embodiments, the cosmetic composition as otherwise described herein comprises a surfactant system comprising a plurality of different surfactants. In certain embodiments, the surfactant system comprises a surfactant selected from the group consisting of anionic surfactants, cationic surfactants, non-ionic surfactants, zwitterionic surfactants and/or amphoteric surfactants. In certain embodiments, the surfactant is selected from the group consisting of anionic surfactants, cationic surfactants, non-ionic surfactants, zwitterionic surfactants and/or amphoteric surfactants.

In certain embodiments, the cosmetic composition comprises a total amount of surfactant of from 0.01 wt % to 70 wt %, from 0.1 wt % to 40%, from 1 wt % to 30%, from 2 wt % to 20 wt %.

In certain embodiments, the cosmetic composition as otherwise described herein comprises an anionic surfactant. In certain embodiments, the cosmetic composition comprises an anionic surfactant as cosmetically acceptable component (II). In certain embodiments, the anionic surfactant is selected from the group consisting of (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, acylglutamates, and mixtures thereof. The anionic surfactants (and their mixtures) can be used in the form of their water-soluble or water-dispersible salts, examples being the sodium, potassium, magnesium, ammonium, mono-, di-, and triethanolammonium, and analogous alkylammonium salts. In certain embodiments, the anionic surfactant is the salt of an anionic surfactant comprising 12 to 14 carbon atoms. In certain embodiments, the anionic surfactant is selected from the group consisting of sodium lauryl sulfate, sodium laureth sulfate, sodium tridecyl sulfate, sodium trideceth sulfate, sodium myristyl sulfate, sodium myreth sulfate, and mixtures thereof. Typical anionic surfactants for use in compositions of the invention include sodium oleyl succinate, ammonium lauryl sulphosuccinate, sodium lauryl sulphate, sodium lauryl ether sulphate, sodium lauryl ether sulphosuccinate, ammonium lauryl sulphate, ammonium lauryl ether sulphate, sodium dodecyl benzene sulphonate, triethanolamine dodecylbenzene sulphonate, sodium cocoyl isethionate, sodium lauryl isethionate, lauryl ether carboxylic acid and sodium N-lauryl sarcosinate. Preferred anionic surfactants are selected from sodium lauryl sulphate and sodium lauryl ether sulphate(n)EO, (where n is from 1 to 3); more preferably sodium lauryl ether sulphate(n)EO, (where n is from 1 to 3); most preferably sodium lauryl ether sulphate(n)EO where n=1. Preferably the level of alkyl ether sulphate is from 0.5 wt % to 25 wt % of the total composition, more preferably from 3 wt % to 18 wt %, most preferably from 6 wt % to 15 wt % of the total composition.

The total amount of anionic surfactant in the composition may range from 0.5 wt % to 45 wt %, more preferably from 1.5 wt % to 20 wt %.

In certain embodiments, the cosmetic composition as otherwise described herein comprises a fatty acyl isethionate. In certain embodiments, the cosmetic composition comprises fatty acyl isethionate at a level of from 1 to 10 wt %, more preferably from 2 to 8 wt %, most preferably from 2.5 to 7.5 wt %. A preferred fatty acyl isethionate product comprises fatty acyl isethionate surfactant at a level of from 40 to 80 wt % of the product, as well as free fatty acid and/or fatty acid salt at a level of from 15 to 50 wt. %. Preferably, greater than 20 wt % and less than 45 wt %, more preferably greater than 25 wt % and less than 45 wt % of the fatty acyl isethionate are of chain length greater than or equal to C6; and greater than 50 wt %, preferably greater than 60 wt % of the free fatty acid/soap is of chain length C₆ to C₂₀. In addition, the composition may contain isethionates salts which are present typically at levels less than 5 wt %, and traces (less than 2 wt %) of other impurities. Preferably, a mixture of aliphatic fatty acids is used for the preparation of commercial fatty acyl isethionates surfactants. The resulting fatty acyl isethionate surfactants (e.g., resulting from reaction of alkali metal isethionate and aliphatic fatty acid) preferably should have more than 20 wt %, preferably more than 25 wt %, but no more than 45 wt %, preferably 35% (on basis of fatty acyl isethionates reaction product) of fatty acyl group with 16 or greater carbon atoms to provide both excellent lather and mildness of the resulting fatty acyl isethionate product. These longer chain fatty acyl isethionate surfactants and fatty acids, i.e. fatty acyl group and fatty acid with 16 or more carbons, can typically form insoluble surfactant/fatty acid crystals in water at ambient temperatures.

In certain embodiments, the cosmetic composition as otherwise described herein comprises an acylglycinate surfactant. In certain embodiments, the acylglycinate surfactant conforms to the formula (Y):

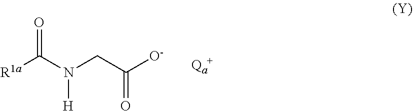

wherein
R^{1a} is a linear or branched, saturated alkanoyl group having 6 to 30, preferably 8 to 22, particularly preferably 8 to 18, carbon atoms or is a linear or branched, mono- or polyunsaturated alkenoyl group having 6 to 30, preferably 8 to 22 and particularly preferably 12 to 18 carbon atoms, and Qₐ⁺ is a cation. In certain embodiments, Qₐ⁺ is selected from the group consisting of Li⁺, Na⁺, K⁺, Mg′, Ca⁺⁺, Al⁺⁺⁺, NH₄⁺, a monoalkylammmonium ion, a dialkylammonium ion, a trialkylammonium ion and a tetraalkylammonium ion, or combinations thereof.

Optionally R^{1a} is independently from one another, are (C₁-C₂₂)-alkyl radicals or (C₂-C₁₀)-hydroxyalkyl radicals. In certain embodiments, the acylglycinate surfactant is selected from sodium cocoylglycinate and potassium cocoylglycinate. In certain embodiments, the acylglycinate surfactant is selected from those conforming to formula (Y), wherein R is C₁₂ alkyl or C₁₄ alkyl. In certain embodiments, the acylglycinate surfactant is selected from those conforming to formula (Y), wherein R is C₁₆ alkyl or C₁₈ alkyl.

In certain embodiments, the cosmetic composition as otherwise described herein comprises from 0.01 wt % to 30 wt %, or 1 wt % to 25 wt %, preferably from 5 wt % to 20 wt %, more preferably from 12 wt % to 18 wt % acylglycinate surfactant.

In certain embodiments, the cosmetic composition as otherwise described herein comprises a glutamate surfactant corresponding to formula (Z) or a salt thereof:

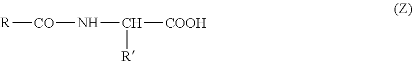

wherein
R′ is HOOC—CH₂—CH₂— or M⁺-OOC—CH₂—CH₂— wherein M⁺ is a cation; and wherein
R is a linear or branched, saturated alkanoyl group having 6 to 30, preferably 8 to 22, more preferably 8 to 18, carbon atoms or is a linear or branched, mono- or polyunsaturated alkenoyl group having 6 to 30, preferably 8 to 22 and more preferably 12 to 18 carbon atoms. In certain embodiments, M⁺ is a metal cation. In certain embodiments, M⁺ is selected from the group consisting of Li⁺, Na⁺, K⁺, Mg⁺⁺, Ca⁺⁺, Al⁺⁺⁺, NH₄⁺, a monoalkylammmonium ion, a dialkylammonium ion, a trialkylammonium ion and a tetraalkylammonium ion, or combinations thereof. In certain embodiments, the glutamate surfactant is selected from sodium cocoyl glutamate and potassium cocoyl glutamate. In certain embodiments, the glutamate surfactant is selected from those conforming to formula (Z), wherein R is C₁₂ alkyl or C₁₄ alkyl. In certain embodiments, the glutamate surfactant is selected from those conforming to formula (Z), wherein R is C₁₆ alkyl or C₁₈ alkyl.

In certain embodiments, the cosmetic composition as otherwise described herein comprises from 0.01 wt % to 30 wt %, or 1 wt % to 25 wt %, preferably from 5 wt % to 20 wt %, more preferably from 12 wt % to 18 wt % glutamate surfactant.

In certain embodiments, the cosmetic composition as otherwise described herein comprises a non-ionic surfactant. The non-ionic surfactants may be present in the range 0 to 5 wt %. The non-ionic surfactants that can be included in the compositions herein include condensation products of aliphatic primary or secondary linear or branched chain alcohols or phenols with alkylene oxides, usually ethylene oxide and generally having from 6 to 30 ethylene oxide groups. Alkyl ethoxylates are particularly preferred. Most preferred are alky ethoxylates having the formula

R—(OCH₂CH₂)ₙOH

where
R is an alkyl chain of C₁₂ to C₁₅, and
n is 5 to 9.

Other suitable nonionic surfactants include mono- or di-alkyl alkanolamides. Examples include coco mono- or di-ethanolamide and coco mono-isopropanolamide.

Further nonionic surfactants which can be included in compositions of the invention are the alkyl polyglycosides (APGs). Typically, APG is one which comprises an alkyl group connected (optionally via a bridging group) to a block of one or more glycosyl groups. Preferred APGs are defined by the following formula:

RO-(G)n

wherein
n in an integer of about 1 to about 50;
R is a branched or straight chain alkyl group which may be saturated or unsaturated; and
G is a saccharide group.

In certain embodiments, R may represent a mean alkyl chain length of from about C₅ to about C₂₀.

Preferably R represents a mean alkyl chain length of from about C₉ to about C₁₂. G may be selected from C₅ or C₆ monosaccharide residues, and is preferably a glucoside. G may be selected from the group comprising glucose, xylose, lactose, fructose, mannose and derivatives thereof. Preferably G is glucose. The degree of polymerisation, n, may have a value of from about 1 to about 10 or more. Most preferably the value of n lies from about 1.3 to about 1.5. Suitable alkyl polyglycosides for use in the invention are commercially available and include for example those materials identified as: Oramix NS10 ex Seppic; Plantaren 1200 and Plantaren 2000 ex Henkel.

In certain embodiments, the non-ionic surfactant has an HLB (Hydrophilic Lipophilic Balance) of greater than 12. Optionally, the non-ionic surfactant is selected from the group consisting of ethoxylated or ethoxylated/propoxylated fatty alcohols with a fatty chain comprising from 12 to 22 carbon atoms, ethoxylated sterols, such as stearyl- or lauryl alcohol (EO-7), PEG-16 soya sterol or PEG-10 soya sterol, polyoxyethylene polyoxypropylene block polymers (poloxamers), and mixtures thereof.

In certain embodiments, the non-ionic surfactant is selected from the group consisting of ethoxylated fatty alcohols, fatty acids, fatty acid glycerides or alkylphenols, in particular addition products of from 2 to 30 mol of ethylene oxide and/or 1 to 5 mol of propylene oxide onto C₈- to C₂₂-fatty alcohols, onto C₁₂- to C₂₂-fatty acids or onto alkyl phenols having 8 to 15 carbon atoms in the alkyl group, C₁₂- to C₂₂-fatty acid mono- and diesters of addition products of from 1 to 30 mol of ethylene oxide onto glycerol, addition products of from 5 to 60 mol of ethylene oxide onto castor oil or onto hydrogenated castor oil, fatty acid sugar esters, in particular esters of sucrose and one or two C₈- to C₂₂-fatty acids, INCI: Sucrose Cocoate, Sucrose Dilaurate, Sucrose Distearate, Sucrose Laurate, Sucrose Myristate, Sucrose Oleate, Sucrose Palmitate, Sucrose Ricinoleate, Sucrose Stearate, esters of sorbitan and one, two or three C₈- to C₂₂-fatty acids and a degree of ethoxylation of from 4 to 20, polyglyceryl fatty acid esters, in particular of one, two or more C₈- to C₂₂-fatty acids and polyglycerol having preferably 2 to 20 glyceryl units, alkyl glucosides, alkyl oligoglucosides and alkyl polyglucosides having C₈ to C₂₂-alkyl groups, e.g. decylglucoside or laurylglucoside, and mixtures thereof.

In certain embodiments, the non-ionic surfactant is selected from the group consisting of fatty alcohol ethoxylates (alkylpolyethylene glycols), alkylphenol polyethylene glycols, alkylmercaptan polyethylene glycols, fatty amine ethoxylates (alkylaminopolyethylene glycols), fatty acid ethoxylates (acylpolyethylene glycols), polypropylene glycol ethoxylates (Pluronics®), fatty acid alkylol amides, (fatty acid amide polyethylene glycols), N-alkyl-, N-alkoxypolyhydroxy-fatty acid amide, sucrose esters, sorbitol esters, polyglycol ethers, and mixtures thereof.

Other sugar-derived nonionic surfactants which can be included in compositions of the invention include the fatty (e.g. C₁₀-C₁₈)N-alkyl (C₁-C₆) polyhydroxy fatty acid amides, such as the C₁₂-C₁₈ N-methyl glucamides, as described for example in WO1992006154A1 (Mao, P&G), and the N-alkoxy polyhydroxy fatty acid amides. In certain embodiments, the cosmetic composition as otherwise described herein comprises a fatty N-methyl-N-glucamide surfactant. In certain embodiments, the fatty N-methyl-N-glucamide surfactant conforms to the formula (X):

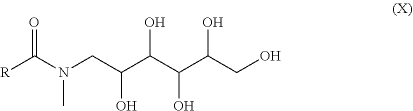

wherein
R is a linear or branched alkyl or alkenyl group having from 3 to 30 carbon atoms. In certain embodiments, R is an alkyl group having from 3 to 30 carbon atoms. In certain embodiments, R is a saturated aliphatic hydrocarbon group which can be linear or branched and can have from 3 to 20 carbon atoms in the hydrocarbon chain, preferably linear or branched. Branched means that a lower alkyl group such as methyl, ethyl or propyl is present as substituent on a linear alkyl chain. In certain embodiments, R is selected from the group consisting of 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl and 1-octadecyl. Suitable fatty N-methyl-N-glucamide surfactants are described in Klug et al. WO2013178700A2 (Clariant) and Connor et al. EP0550637B1 (P&G), which are incorporated herein by reference. In certain embodiments, the fatty N-methyl-N-glucamide surfactant is selected from those conforming to formula (X), wherein R is C₁₂ alkyl or C₁₄ alkyl. In certain embodiments, the fatty N-methyl-N-glucamide surfactant is selected from those conforming to formula (X), wherein R is C₁₆ alkyl or C₁₈ alkyl.

In certain embodiments, the cosmetic composition as otherwise described herein comprises from 1 wt % to 20 wt %, more preferably from 2 wt % to 10 wt %, even more preferably from 3 wt % to 7 wt % non-ionic surfactant.

In certain embodiments, the cosmetic composition as otherwise described herein comprises from 1 wt % to 20 wt %, more preferably from 2 wt % to 10 wt %, even more preferably from 3 wt % to 7 wt % fatty N-methyl-N-glucamide surfactant.

Amphoteric or zwitterionic surfactant(s) can be included in the composition in an amount ranging from 0.5 wt % to about 8 wt %, preferably from 1 wt % to 4 wt % of the total composition.

In certain embodiments, the amphoteric surfactants are selected from the group consisting of N—(C₁₂-C₁₈)-alkyl-β-aminopropionates and N—(C₁₂-C₁₈)-alkyl-β-iminodipropionates as alkali metal salts and mono-, di-, and trialkylammonium salts; N-acylaminoalkyl-N,N-dimethylacetobetaine, preferably N—(C₈-C₁₈)-acylaminopropyl-N,N-dimethylacetobetaine, (C₁₂-C₁₈)-alkyl-dimethylsulfopropylbetaine, amphosurfactants based on imidazoline (trade name: Miranol®, Steinapoe), preferably the sodium salt of 1-(β-carboxymethyloxyethyl)-1-(carboxymethyl)-2-laurylimidazolinium; amine oxide, e.g., (C₁₂-C₁₈)-alkyl-dimethylamine oxide, fatty acid amidoalkyldimethylamine oxide, and mixtures thereof.

In certain embodiments, the cosmetic composition as otherwise described herein comprises a betaine surfactant. Optionally, the betaine surfactant is selected from C₈- to C₁₈-alkylbetaines. In certain embodiments, the betaine surfactant is selected from the group consisting of cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and laurylbis(2-hydroxypropyl)alphacarboxyethylbetaine and combinations thereof. Optionally, the betaine surfactant is selected from C₈- to C₁₈-sulfobetaines. In certain embodiments, the betaine surfactant is selected from the group consisting of cocodimethylsulfopropylbetaine, stearyldimethylsulfopropylbetaine, lauryldimethyl-sulfoethylbetaine, laurylbis(2-hydroxyethyl)sulfopropylbetaine, and combinations thereof. Optionally, the betaine surfactant is selected from carboxyl derivatives of imidazole, the C₈- to C₁₈-alkyldimethylammonium acetates, the C₈- to C₁₈-alkyldimethylcarbonylmethylammonium salts, and the C₈- to C₁₈-fatty acid alkylamidobetaines, and mixtures thereof. Optionally, the C₈- to C₁₈-fatty acid alkylamidobetaine is selected from coconut fatty acid amidopropylbetaine, N-coconut fatty acid amidoethyl-N-[2-(carboxymethoxy)ethyl]glycerol (CTFA name: Cocoamphocarboxyglycinate), and mixtures thereof. A particularly preferred amphoteric or zwitterionic surfactant is cocamidopropyl betaine. Mixtures of any of the foregoing amphoteric or zwitterionic surfactants may also be suitable. Preferred mixtures are those of cocamidopropyl betaine with further amphoteric or zwitterionic surfactants as described above. A preferred further amphoteric or zwitterionic surfactant is sodium cocoamphoacetate.

In certain embodiments, the cosmetic composition as otherwise described herein comprises from 0.5 wt % to 20 wt %, preferably from 1 wt % to 10 wt % amphoteric surfactant.

In certain embodiments, the cosmetic composition as otherwise described herein comprises a surfactant system. In certain embodiments, the surfactant system comprises at least one surfactant selected from the group consisting of lauryl sulfate, laureth sulfate, cocoamidopropylbetaine, sodium cocoylglutamate, lauroamphoacetate, and mixtures thereof. In certain embodiments, the surfactant system comprises sodium laureth sulphate, sodium lauryl sulphate, and optionally cocamidopropyl betaine. In certain embodiments, the surfactant system comprises sodium laureth sulphate, Potassium Cocyl Glutamate, and cocamidopropyl betaine.

### Auxiliary

In certain embodiments, the cosmetic composition as otherwise described herein comprises at least one additive common in cosmetology, pharmacy, and dermatology, which are hereinafter called auxiliaries. In certain embodiments, the composition comprises an auxiliary. In certain embodiments, the auxiliary is cosmetically acceptable. In certain embodiments, the auxiliary is selected from the group consisting of oily substances, waxes, emulsifiers, coemulsifiers, solubilizers, cationic polymers, film formers, superfatting agents, refatting agents, foam stabilizers, stabilizers, active biogenic substances, preservatives, preservation boosting ingredients, anti-fungal substances, anti-dandruff agents, dyes or pigments, particulate substances, opacifiers, abrasives, absorbents, anticaking agents, bulking agents, pearlizing agents, direct dyes, perfumes or fragrances, carriers, solvents or diluents, propellants, functional acids, active ingredients, skin-brightening agents, self-tanning agents, exfoliants, enzymes, anti-acne agents, deodorants and anti-perspirants, viscosity modifiers, thickening and gelling agents, pH adjusting agents, buffering agents, anti-oxidants, chelants, astringents, sunscreens, sun protection agents, UV filters, skin conditioning agents, emollients, humectants, occlusive agents, pediculocides, anti-foaming agents, flavouring agents, electrolytes, oxidizing agents and reducing agents.

In certain embodiments, the cosmetic composition as otherwise described herein comprises an oily substance or wax. In certain embodiments, the cosmetic composition as otherwise described herein comprises an oily substance or wax, wherein the oily substance or wax are selected from the group consisting of silicone oils, volatile or nonvolatile, linear, branched or cyclic, optionally with organic modification; phenylsilicones; silicone resins and silicone gums; mineral oils such as paraffin oil or vaseline oil; oils of animal origin such as perhydrosqualene, lanolin; oils of plant origin such as liquid triglycerides, e.g., sunflower oil, corn oil, soybean oil, rice oil, jojoba oil, babassu oil, pumpkin oil, grapeseed oil, sesame oil, walnut oil, apricot oil, macadamia oil, avocado oil, sweet almond oil, lady's-smock oil, castor oil, triglycerides of caprylic/capric acids, olive oil, peanut oil, rapeseed oil, argan oil, abyssinian oil, and coconut oil; synthetic oils such as purcellin oil, isoparaffins, linear and/or branched fatty alcohols and fatty acid esters, preferably guerbet alcohols having 6 to 18, preferably 8 to 10, carbon atoms; esters of linear (C₆-C₁₃) fatty acids with linear (C₆-C₂₀) fatty alcohols; esters of branched (C₆-C₁₃) carboxylic acids with linear (C₆-C₂₀) fatty alcohols, esters of linear (C₆-C₁₈) fatty acids with branched alcohols, especially 2-ethylhexanol; esters of linear and/or branched fatty acids with polyhydric alcohols (such as dimerdiol or trimerdiol, for example) and/or guerbet alcohols; triglycerides based on (C₆-C₁₀) fatty acids; esters such as dioctyl adipate, diisopropyl dimer dilinoleate; propylene glycols/dicaprylate or waxes such as beeswax, paraffin wax or microwaxes, alone or in combination with hydrophilic waxes, such as cetylstearyl alcohol, for example; fluorinated and perfluorinated oils; fluorinated silicone oils; mixtures of the aforementioned compounds.

In certain embodiments, the cosmetic composition as otherwise described herein comprises an oily substance, which is any fatty substance which is liquid at room temperature (25° C.). In a preferred embodiment, the oily substance is selected from the group consisting of sweet almond oil, caprylic/capric triglycerides, dimethicone, mineral oil, squalane, castor oil, isopropyl isostearate, jojoba oil, dicaprylyl carbonate, isohexadecane, C₁₂-C₁₅ alkyl benzoate, and combinations thereof. In certain embodiments, the cosmetic composition as otherwise described herein comprises from 0.001 wt % to 60 wt %, preferably from 0.05 wt % to 50 wt %, even more preferably from 0.1 wt % to 40 wt % of at least one oily substance.

In a preferred embodiment, the wax is selected from the group consisting of carnauba wax, beeswax, candelilla wax, synthetic wax, polyethylene, paraffin wax, microcrystalline wax, hydrogenated vegetable oil, hydrogenated castor oil, rice bran wax, cetyl dimethicone, bis-PEG-18 methyl ether dimethyl silane, and combinations thereof. In certain embodiments, the cosmetic composition as otherwise described herein comprises from 0.001 wt % to 30 wt %, preferably from 0.05 wt % to 20 wt %, even more preferably from 0.1 wt % to 10 wt % of at least one wax.

In certain embodiments, the cosmetic composition as otherwise described herein comprises an emulsifier, coemulsifier or solubilizer. Non-ionic, anionic, cationic or amphoteric surface active compounds can be used as emulsifiers, coemulsifiers and solubilizers.

As nonionogenic surface active compounds, consideration may preferably be given to: addition products of 0 to 30 mol ethylene oxide and/or 0 to 5 mol propylene oxide on linear fatty alcohols with 8 to 22 carbon atoms, on fatty acids with 12 to 22 carbon atoms, on alkyl phenols with 8 to 15 carbon atoms in the alkyl group and on sorbitan or sorbitol esters; (C₁₂-C₁₈)-fatty acid mono- and diesters of addition products of 0 to 30 mol ethylene oxide on glycerol; glycerol mono- and diesters and sorbitan mono- and diesters of saturated and unsaturated fatty acids with 6 to 22 carbon atoms and optionally their ethylene oxide addition products; addition products of 15 to 60 mol ethylene oxide on castor oil and/or hardened castor oil; polyol and especially polyglycerol esters, e.g. polyglycerol polyricinoleate and polyglycerol poly-12-hydroxystearate. Ethoxylated fatty amines, fatty acid amides, fatty acid alkanolamides and mixtures of compounds from several of these classes of substances are also preferably suitable. Polymeric ethers formed by block polymerization of ethylene or propylene oxide known as poloxamers are also suitable.

Suitable ionogenic coemulsifiers are e.g. anionic emulsifiers, such as mono-, di- or triphosphoric acid esters, soaps (e.g. sodium stearate), fatty alcohol sulfates as well as cationic emulsifiers such as mono-, di- and tri-alkyl quats and polymeric derivatives thereof.

Amphoteric emulsifiers that are available are preferably alkyl aminoalkyl carboxylic acids, betaines, sulfobetaines and imidazoline derivatives.

Fatty alcohol ethoxylates are used especially preferably, and may be selected from the group consisting of ethoxylated stearyl alcohols, isostearyl alcohols, cetyl alcohols, isocetyl alcohols, oleyl alcohols, lauryl alcohols, isolauryl alcohols and cetylstearyl alcohols, especially polyethylene glycol (13) stearyl ether, polyethylene glycol (14) stearyl ether, polyethylene glycol (15) stearyl ether, polyethylene glycol (16) stearyl ether, polyethylene glycol (17) stearyl ether, polyethylene glycol (18) stearyl ether, polyethylene glycol (19) stearyl ether, polyethylene glycol (20) stearyl ether, polyethylene glycol (12) isostearyl ether, polyethylene glycol (13) isostearyl ether, polyethylene glycol (14) isostearyl ether, polyethylene glycol (15) isostearyl ether, polyethylene glycol (16) isostearyl ether, polyethylene glycol (17) isostearyl ether, polyethylene glycol (18) isostearyl ether, polyethylene glycol (19) isostearyl ether, polyethylene glycol (20) isostearyl ether, polyethylene glycol (13) cetyl ether, polyethylene glycol (14) cetyl ether, polyethylene glycol (15) cetyl ether, polyethylene glycol (16) cetyl ether, polyethylene glycol (17) cetyl ether, polyethylene glycol (18) cetyl ether, polyethylene glycol (19) cetyl ether, polyethylene glycol (20) cetyl ether, polyethylene glycol (13) isocetyl ether, polyethylene glycol (14) isocetyl ether, polyethylene glycol (15) isocetyl ether, polyethylene glycol (16) isocetyl ether, polyethylene glycol (17) isocetyl ether, polyethylene glycol (18) isocetyl ether, polyethylene glycol (19) isocetyl ether, polyethylene glycol (20) isocetyl ether, polyethylene glycol (12) oleyl ether, polyethylene glycol (13) oleyl ether, polyethylene glycol (14) oleyl ether, polyethylene glycol (15) oleyl ether, polyethylene glycol (12) lauryl ether, polyethylene glycol (12) isolauryl ether, polyethylene glycol (13) cetylstearyl ether, polyethylene glycol (14) cetylstearyl ether, polyethylene glycol (15) cetylstearyl ether, polyethylene glycol (16) cetylstearyl ether, polyethylene glycol (17) cetylstearyl ether, polyethylene glycol (18) cetylstearyl ether, polyethylene glycol (19) cetylstearyl ether.

Fatty acid ethoxylates are also preferred, and may be selected from the group consisting of ethoxylated stearates, isostearates and oleates, especially polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

Sodium laureth-11-carboxylate can be used advantageously as ethoxylated alkyl ether carboxylic acid or salts thereof.

Polyethylene glycol (60) evening primrose glycerides can be used advantageously as ethoxylated triglycerides.

Furthermore, it is advantageous to select the polyethylene glycol glycerol fatty acid ester from the group polyethylene glycol (20) glyceryl laurate, polyethylene glycol (6) glyceryl caprate/caprinate, polyethylene glycol (20) glyceryl oleate, polyethylene glycol (20) glyceryl isostearate and polyethylene glycol (18) glyceryl oleate/cocoate.

Among the sorbitan esters, the following are especially suitable: polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

Especially advantageous coemulsifiers are glyceryl monostearate, glyceryl monooleate, diglyceryl monostearate, glyceryl isostearate, polyglyceryl-3-oleate, polyglyceryl-3-diisostearate, polyglyceryl-4-isostearate, polyglyceryl-2-dipolyhydroxystearate, polyglyceryl-4-di polyhydroxystearate, PEG-30-dipolyhydroxystearate, diisostearoylpolyglyceryl-3-diisostearate, glycol distearate and polyglyceryl-3-dipolyhydroxystearate, sorbitan monoisostearate, sorbitan stearate, sorbitan oleate, sucrose distearate, lecithin, PEG-7-hydrogenated castor oil, cetyl alcohol, stearyl alcohol, behenyl alcohol, isobehenyl alcohol and polyethylene glycol (2) stearyl ether (steareth-2), alkyl methicone copolyols and alkyl dimethicone copolyols, especially cetyldimethicone copolyol (ABIL® EM 90) or laurylmethicone copolyol.

In a preferred embodiment, the emulsifier, coemulsifier or solubilizer is selected from the group consisting of glyceryl stearate, cetearyl alcohol, polysorbate 20, stearic acid, cetearyl glucoside, PEG-40 hydrogenated castor oil, cetyl phosphate, steareth-2, ceteth-10 phosphate, trilaureth-4 phosphate, polyglyceryl-2 sesquiisostearate, cetyl PEG/PPG-10/1 dimethicone, cetrimonium chloride, and combinations thereof.

In certain embodiments, the cosmetic composition as otherwise described herein comprises from 0.1 wt % to 20 wt %, preferably from 0.5 wt % to 10 wt %, even more preferably from 1.0 wt % to 5.0 wt % of at least one emulsifier, coemulsifier and/or solubilizer.

In certain embodiments, the cosmetic composition as otherwise described herein comprises a cationic polymer. Suitable cationic polymers include those known under the INCI designation “polyquaternium”, especially polyquaternium-31, polyquaternium-16, polyquaternium-24, Polyquaternium-7, polyquaternium-22, polyquaternium-39, polyquaternium-28, polyquaternium-2, polyquaternium-10, polyquaternium-11, and also polyquaternium-37 & mineral oil & PPG trideceth (Salcare SC95), PVP-dimethylaminoethyl methacrylate copolymer, guar-hydroxypropyltriammonium chlorides, and also calcium alginate and ammonium alginate. It is additionally possible to employ cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as amidomethicones, for example; copolymers of adipic acid and dimethylaminohydroxypropyldiethylenetriamine; polyaminopolyamide and cationic chitin derivatives, such as chitosan, for example.

In a preferred embodiment, the cationic polymer is selected from the group consisting of polyquaternium-10, guar hydroxypropyltrimonium chloride, polyquaternium-7, polyquaternium-6, and combinations thereof.

In certain embodiments, the cosmetic composition as otherwise described herein comprises from 0.1 wt % to 10 wt %, preferably from 0.5 wt % to 7.5 wt %, even more preferably from 1.0 wt % to 5.0 wt % of at least one cationic polymer.

In certain embodiments, the cosmetic composition as otherwise described herein comprises a film former. Film formers are materials which produce a continuous film on skin, hair, or nails such as synthetic or natural polymers and their derivatives. The compositions according to the invention can contain film formers, which are, depending on the intended use, selected from salts of phenylbenzimidazole sulfonic acid, water-soluble polyurethanes, for example C10-polycarbamyl polyglyceryl ester, polyvinyl alcohol, polyvinylpyrrolidone (PVP) copolymers, vinylpyrrolidone/vinyl acetate copolymer or PVP/eicosene copolymers, vinylpyrrolidone/alkene copolymers, for example VP/eicosene copolymer or VP/hexadecene copolymer, PVM/MA copolymer or esters thereof, maleinized polypropylene polymers, water-soluble acrylic acid polymers/copolymers or esters or salts thereof, for example partial-ester copolymers of acrylic/methacrylic acid, polyalkylsilsesquioxanes, polyacrylamide, water-soluble cellulose, for example hydroxymethylcellulose, hydroxyethylcellulose, hydroxypropylcellulose, water-soluble quaterniums, polyquaterniums, carboxyvinyl polymers, such as carbomers and salts thereof, polysaccharides, for example polydextrose and glucan, vinyl acetate/crotonate.

In a preferred embodiment, the film former is selected from the group consisting of VP/eicosene copolymer, PVP, VPNA copolymer, styrene/acrylates copolymer, acrylates copolymer, butyl ester of PVM/MA copolymers, hydroxyethylcellulose, polyquaternium-10, polypropylsilsesquioxane, polyurethane-64, and combinations thereof.

In certain embodiments, the cosmetic composition as otherwise described herein comprises from 0.1 wt % to 10 wt %, preferably from 0.5 wt % to 7.5 wt %, even more preferably from 1.0 wt % to 5.0 wt % of at least one film former.

In certain embodiments, the cosmetic composition as otherwise described herein comprises a superfatting agent and/or a refatting agent. As superfatting agents it is possible to use substances such as, for example, lanolin, polyethoxylated lanolin derivatives, lecithin, lecithin derivatives, non-ethoxylated and polyethoxylated or acylated lanolin and lecithin derivatives, polyol fatty acid esters such as glyceryl oleate, mono-, di- and triglycerides and/or fatty acid alkanolamides, can preferably be used as overfatting agents or refatting agents. These compounds can also simultaneously serve as foam stabilizers. In a preferred embodiment, the superfatting agent and/or a refatting agent is selected from the group consisting of, lanolin, glyceryl ricinoleate, PEG-8 glyceryl laurate, glyceryl oleate, cocamide MEA, PEG-75 lanolin, and combinations thereof. In certain embodiments, the cosmetic composition as otherwise described herein comprises from 0.01 wt % to 10 wt %, preferably from 0.1 wt % to 5.0 wt %, even more preferably from 0.5 wt % to 3.0 wt % of at least one superfatting agent and/or a refatting agent.

In certain embodiments, the cosmetic composition as otherwise described herein comprises a stabiliser. As stabiliser it is possible to use metal salts of fatty acids, such as magnesium, aluminum and/or zinc stearate, for example. In a preferred embodiment, the stabilizer is selected from the group consisting of, aluminum stearate, aluminum isostearates/myristates, magnesium stearate, magnesium cocoate, zinc palmitate, zinc stearate, and combinations thereof. In certain embodiments, the cosmetic composition as otherwise described herein comprises from 0.01 wt % to 10 wt %, preferably from 0.5 wt % to 8.0 wt %, even more preferably from 1.0 wt % to 5.0 wt % of at least one stabilizer.

In certain embodiments, the cosmetic composition as otherwise described herein comprises a biogenic substance. In certain embodiments, the cosmetic composition as otherwise described herein comprises a biogenic substance, wherein such substances are selected from plant extracts (e.g. leaf, root, seed, flower and/or stem extracts from aloe vera, camomile, green tea, hamamelis or licorice), local anesthetics, antibiotics, antiphlogistics, antiallergic agents, hormones, beta-glucans, cholesterol, amino acids, ceramides, corticosteroids, sebostatics, Bisabolol®, allantoin, Phytantriol®, proteins, vitamins selected from niacin, biotin, vitamin B2, vitamin B3, vitamin B6, vitamin B5, vitamin B3 derivatives (salts, acids, esters, amides, alcohols), vitamin C and vitamin C derivatives (salts, acids, esters, amides, alcohols), preferably as sodium salt of the monophosphoric acid ester of ascorbic acid or as magnesium salt of the phosphoric acid ester of ascorbic acid, tocopherol and tocopherol acetate, vitamin E and/or its derivatives, protein derivatives such as gelatin, collagen hydrolysates, polypeptides, egg yolk, lecithin, hydrolyzed silk, hydrolyzed keratin, milk protein, cerebrosides or phospholipids. In a preferred embodiment, the biogenic active substance is selected from the group consisting of, aloe vera extract, collagen hydrolysates, bisabolol, vitamin C, vitamin E, allantoin, vitamin B5, tocopherol acetate, retinyl palmitate, and combinations thereof. In certain embodiments, the cosmetic composition as otherwise described herein comprises from 0.001 wt % to 5.0 wt %, preferably from 0.01 wt % to 3.0 wt %, even more preferably from 0.1 wt % to 2.0 wt % of at least one biogenic active substance.

In certain embodiments, the cosmetic composition as otherwise described herein comprises a preservative, preservation boosting ingredient, anti-fungal agent, and/or anti-dandruff agent. In certain embodiments, the preservative is selected from the group consisting of benzyl alcohol, Piroctone Olamine, phenoxyethanol, parabens, pentanediol, benzoic acid/sodium benzoate, sorbic acid/potassium sorbate, and combinations thereof. Other organic acids can also be used to provide antimicrobial protection. In certain embodiments, the preservation boosting ingredient is selected from the group consisting of anisic acid, lactic acid, sorbitan caprylate, ethylhexylglycerin, caprylyl glycol, octanediol, and mixtures thereof. A suitable preservation boosting ingredient is also disclosed in International patent application WO2018002100A1 (PCT/EP2017/065927) by Clariant International Ltd (see in particular claim 1 therein), which is incorporated herein by reference. In certain embodiments, the cosmetic composition as otherwise described herein comprises 0.01 to 5.0 wt %, particularly preferably from 0.05 wt % to 1.0 wt % of at least one preservative. Suitable preservatives include the substances listed in the International Cosmetic Ingredient Dictionary and Handbook, 9^{th} Edition with the function “preservatives”. In certain embodiments, the preservative is selected from the group consisting of phenoxyethanol, benzyl paraben, butyl paraben, ethyl paraben, isobutyl paraben, isopropyl paraben, methyl paraben, propyl paraben, iodopropynyl butylcarbamate, methyldibromoglutaronitrile, DMDM hydantoin and combinations thereof. In certain embodiments, the cosmetic composition as otherwise described herein comprises a preservative selected from the group consisting of cetyltrimethyl ammonium chloride, cetylpyridinium chloride, benzethonium chloride, diisobutylethoxyethyldimethyl benzylammonium chloride, sodium N-lauryl sarcosinate, sodium-N-palmethyl sarcosinate, lauroyl sarcosine, N-myristoylglycine, potassium-N-laurylsarcosine, trimethylammonium chloride, sodium aluminium chlorohydroxylactate, triethylcitrate, tricetylmethylammonium chloride, 2,4,4′-trichloro-2′-hydroxydiphenylether (Triclosan), phenoxyethanol, 1,5-pentandiol, 1,6-hexandiol, 3,4,4′-trichlorocarbanilide (Triclocarban), diaminoalkylamide, L-lysine hexadecylamide, heavy metal citrate salts, salicylate, piroctose, zinc salts, pyrithione and its heavy metal salts, zinc pyrithione, zinc phenol sulfate, farnesol, ketoconazol, oxiconazol, bifonazole, butoconazole, cloconazole, clotrimazole, econazole, enilconazole, fenticonazole, isoconazole, miconazole, sulconazole, tioconazole, fluconazole, itraconazole, terconazole, naftifine, terbinafine, selenium disulfide, methylchloroisothiazolinone, methylisothiazolinone, methyldibromo glutaronitrile, silver chloride (AgCl), diazolidinyl urea, imidazolidinyl urea, dehydroacetic acid, undecylenic acid, chlorphenesin, proprionic acid, salicylic acid, chloroxylenol, sodium salts of diethylhexylsulfosuccinate, sodiumbenzoate, phenoxyethanol, (RS)-1-(4-chlorophenoxy)-1-imidazol-1-yl-3,3-dimethylbutan-2-one (climbazole), benzyl alcohol, phenoxyisopropanol, parabens such as butyl-, ethyl-, methyl- and propylparaben and their salts, 2-Bromo-2-nitropropane-1,3-diol, polyaminopropyl biguanide, phenoxyisopropanol, iodopropynyl butylcarbamate, benzalkonium chloride, benzethonium chloride, pentandiol, 1,2-octanediol, ethylhexylglycerin, sorbic acid, benzoic acid, lactic acid, imidazolidinyl urea, diazolidinyl urea, dimethylol dimethyl hydantoin (DMDMH), chlorhexidine, sodium salts of hydroxymethyl glycinate, hydroxyethylglycine of sorbic acid, and combinations thereof. In certain embodiments, the preservative is selected from the group consisting of phenoxyethanol, benzyl paraben, butyl paraben, ethyl paraben, isobutyl paraben, isopropyl paraben, methyl paraben, propyl paraben, iodopropynyl butylcarbamate, methyldibromoglutaronitrile, DMDM hydantoin and combinations thereof. In certain embodiments, the composition is substantially free of parabens.

In certain embodiments, the cosmetic composition as otherwise described herein comprises from 0.1 wt % to 5.0 wt % antimicrobial agents. In certain embodiments, the antimicrobial combination composition is chlorhexidine.

In certain embodiments, the cosmetic composition as otherwise described herein comprises an anti-fungal substance. In certain embodiments, the anti-fungal substance is selected from the group consisting of ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine, terbinafine, zinc pyrithione, Piroctone Olamine (Octopirox), (RS)-1-(4-chlorophenoxy)-1-imidazol-1-yl-3,3-dimethylbutan-2-one (climbazole), and combinations thereof. In certain embodiments, the cosmetic composition as otherwise described herein comprises a total amount of anti-fungal substance in the composition of from 0.1 wt % to 1.0 wt %. In certain embodiments, the cosmetic composition as otherwise described herein comprises pyridinethione anti-dandruff particulates, for example 1-hydroxy-2-pyridinethione salts, are highly preferred particulate anti-dandruff agents. The concentration of pyridinethione anti-dandruff particulate may ranges from 0.1% to 4.0%, by weight of the formulation, preferably from 0.1% to 3.0%, more preferably from 0.3% to 2.0%. Preferred pyridinethione salts include those formed from heavy metals such as zinc, tin, cadmium, magnesium, aluminum and zirconium, preferably zinc, more preferably the zinc salt of 1-hydroxy-2-pyridinethione (known as “zinc pyridinethione” or “ZPT”), more preferably 1-hydroxy-2-pyridinethione salts in platelet particle form. Salts formed from other cations, such as sodium, may also be suitable. Pyridinethione anti-dandruff agents are described, for example, in US patents U.S. Pat. Nos. 2,809,971; 3,236,733; 3,761,418; 4,345,080; 4,323,683; 4,379,753; and 4,470,982. It is contemplated that when ZPT is used as the anti-dandruff particulate in the compositions herein, that the growth or regrowth of hair may be stimulated or regulated, or both, or that hair loss may be reduced or inhibited, or that hair may appear thicker or fuller.

In certain embodiments, the cosmetic composition as otherwise described herein comprises a preservative system comprising a plurality of different compounds selected from the group consisting of preservatives, preservation boosting ingredients, anti-fungal agents and anti-dandruff agents.

In certain embodiments, the cosmetic composition as otherwise described herein comprises a dye or pigment. In certain embodiments, the cosmetic composition as otherwise described herein comprises at least one dye or pigment. Suitable dyes and pigments are disclosed in WO2013017262A1 in the table spanning pages 36 to 43. These may be colored pigments which impart color effects to the product mass or to hair or skin, or they may be luster effect pigments which impart luster effects to the product mass or to the hair or skin. The color or luster effects on the hair or skin are preferably temporary, i.e. they last until the next hair or skin wash and can be removed again by washing the hair or skin with customary shampoos, body or face cleansers, body washes etc. or by using a make-up remover, micellar water or cleansing wipes. In certain embodiments, the cosmetic composition as otherwise described herein comprises a total amount of from 0.01 wt % to 25 wt %, preferably from 0.1 wt % to 15 wt %, even more preferably from 0.5 wt % to 10 wt % of at least one pigment. In certain embodiments, the particle size of the pigment is from 1 micron to 200 micron, preferably from 3 micron to 150 micron, more preferably 10 micron to 100 micron. The pigments are colorants which are virtually insoluble in the application medium, and may be inorganic or organic. Inorganic-organic mixed pigments are also possible. Preference is given to inorganic pigments. The advantage of inorganic pigments is their excellent resistance to light, weather and temperature. The inorganic pigments may be of natural origin. In certain embodiments, the inorganic pigment is selected from the group consisting of chalk, ochre, umber, green earth, burnt sienna, graphite, and combinations thereof. The pigments may be white pigments, such as, for example, titanium dioxide or zinc oxide, black pigments, such as, for example, iron oxide black, colored pigments, such as, for example, ultramarine or iron oxide red, lustre pigments, metal effect pigments, pearlescent pigments, and fluorescent or phosphorescent pigments, where preferably at least one pigment is a colored, nonwhite pigment. In certain embodiments, the pigment is selected from the group consisting of metal oxides, hydroxides and oxide hydrates, mixed phase pigments, sulfur-containing silicates, metal sulfides, complex metal cyanides, metal sulfates, chromates and molybdates, and the metals themselves (bronze, silver, gold pigments), and combinations thereof. In certain embodiments, the pigment is selected from the group consisting of titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), Prussian blue (ferric ferrocyanide, CI 77510), carmine (cochineal), and combinations thereof. In certain embodiments, the pigment is selected from the group consisting of pearlescent and colored pigments which consist of either single crystals like mica, silica, bismuth oxychloride, boron nitride or titanium dioxide or which have a layer-substrate structure based on mica, aluminium, aluminium oxide, titanium dioxide, silicium dioxide, silicates (e.g. calcium aluminium borosilicate, calcium sodium borosilicate, magnesium aluminium silicate or sodium magnesium fluorosilicate) which are coated with a metal oxide (e.g. iron oxide, chromium oxide, tin oxide) or a metal oxychloride, such as titanium dioxide or bismuth oxychloride, or a metal hydroxide, such as aluminum hydroxide, and optionally further color-imparting substances, such as Prussian blue, ultramarine, carmine or other organic dyes and where the color can be determined by varying the layer thickness. Such pigments are sold, for example, under the trade names RonaFlair®, Colorona®, Xirona®, and Timiron® by Merck or under the trade name Flamenco®, Timica® and Cloisonne® by BASF, Germany. The pearlescent effect can be controlled both by means of the particle size and by means of the particle size distribution of the pigment population. Suitable particle size distributions are e.g. in the range 2-50 μm, 5-25 μm, 5-40 μm, 5-60 μm, 5-95 μm, 5-100 μm, 10-60 μm, 10-100 μm, 10-125 μm, 20-100 μm, 20-150 μm, and <15 μm. A wider particle size distribution e.g. of 20-150 μm, produces glittering effects, whereas a narrower particle size distribution of <15 μm gives a uniform silky appearance. In a preferred embodiment, the pigment is selected from the group consisting of titanium dioxide (CI 77891), black iron oxide (CI 77499), yellow iron oxide (CI 77492), red and brown iron oxide (CI 77491), mica, silica, bismuth oxychloride, and combinations thereof. In certain embodiments, the pigment is selected from the group consisting of organic pigments such as sepia, gamboge, bone charcoal, Cassel brown, indigo, chlorophyll and other plant pigments. In certain embodiments, the pigment is selected from the group consisting of synthetic organic pigments such as azo pigments, anthraquinoids, indigoids, dioxazine, quinacridone, phthalocyanine, isoindolinone, perylene and perinone, metal complex, alkali blue and diketopyrrolopyrrole pigments.

In certain embodiments, the cosmetic composition as otherwise described herein comprises a particulate substance. In certain embodiments, the composition comprises at least one particulate substance. Suitable substances are, for example, substances which are solid at room temperature (25° C.) and are in the form of particles. Suitable substances are, for example, substances which serve as opacifiers, abrasives, absorbents, anti-caking agents, bulking agents or performance fillers. In certain embodiments, the particulate substance is selected from the group consisting of silica, silicates (e.g. sepiolite, montmorillonite, bentonite, kaolin, hectorite), aluminates, clay earths, mica, talc, starch, perlite, charcoal, pulp powder, seed powder, insoluble salts, in particular insoluble inorganic metal salts, metal oxides (e.g. titanium dioxide), minerals and insoluble polymer particles, such as polyamide derivatives (e.g. nylon-12, nylon-6, polyamide-5), silicones (e.g. polymethylsilsesquioxane), polyesters (e.g. polyester-12), polyethylene and polymethyl methacrylates. The particles are present in the composition in undissolved, preferably stably dispersed form, and, following application to the keratin substrate and evaporation of the solvent, can deposit on the substrate in solid form. A stable dispersion can be achieved by providing the composition with a yield point which is large enough to prevent the solid particles from sinking. An adequate yield point can be established using suitable gel formers in a suitable amount. In certain embodiments, the particulate substance is selected from the group consisting of silica (silica gel, silicon dioxide) and metal salts, in particular inorganic metal salts, where silica is particularly preferred. Metal salts are, for example, alkali metal or alkaline earth metal halides, such as sodium chloride or potassium chloride; alkali metal or alkaline earth metal sulfates, such as sodium sulfate or magnesium sulfate. In a preferred embodiment, the particulate substance is selected from the group consisting of, silica, mica, bentonite, kaolin, talc, polymethylsilsesquioxane, polyethylene, clay, and combinations thereof. In certain embodiments, the composition comprises from 0.01 wt % to 20 wt %, preferably from 0.05 wt % to 15 wt %, even more preferably from 0.5 wt % to 10 wt % of at least one particulate substance.

In certain embodiments, the cosmetic composition as otherwise described herein comprises pearlizing agents. In certain embodiments, the cosmetic composition as otherwise described herein comprises at least one pearlizing agent. In certain embodiments, the particulate substance is selected from the group consisting of, fatty acid monoalkanolamides, fatty acid dialkanolamides, monoesters or diesters of alkylene glycols, especially ethylene glycol and/or propylene glycol or oligomers thereof, with higher fatty acids, e.g. palmitic acid, stearic acid and behenic acid, monoesters or polyesters of glycerol with carboxylic acids, fatty acids and metal salts thereof, ketosulfones or mixtures of the aforementioned compounds. In a preferred embodiment, the pearlizing agent is selected from the group consisting of, ethylene glycol distearates and/or polyethylene glycol distearates with 3 glycol units on average, and combinations thereof. In certain embodiments, the cosmetic composition as otherwise described herein comprises from 0.1 wt % to 15 wt %, preferably from 0.5 wt % to 10 wt %, even more preferably from 1.0 wt % to 5.0 wt % of at least one pearlizing agent.

In certain embodiments, the cosmetic composition as otherwise described herein comprises a direct dye. In certain embodiments, the cosmetic composition as otherwise described herein comprises at least one direct dye. Preferred among the direct dyes are the following compounds, alone or in combination with one another: hydroxyethyl-2-nitro-p-toluidine, 2-hydroxyethylpicramic acid, 4-nitrophenylaminourea, tri(4-amino-3-methylphenyl)carbenium chloride (Basic Violet 2), 1,4-di-amino-9,10-anthracenedione (Disperse Violet 1), 1-(2-hydroxy-ethyl)amino-2-nitro-4-[di(2-hydroxyethyl)amino]benzene (HC Blue No. 2), 4-[ethyl-(2-hydroxyethyl)amino]-1-[(2-hydroxyethyl)amino]-2-nitrobenzene hydrochloride (HC Blue No. 12), 1-amino-4-[di(2-hydroxyethyl)amino]-2-nitrobenzene hydrochloride (HC Red No. 13), 4-amino-1-[(2-hydroxyethyl)amino]-2-nitrobenzene (HC Red No. 3), 4-amino-3-nitrophenol, 4-[(2-hydroxyethyl)amino]-3-nitrophenol, 1-amino-5-chloro-4-[(2,3-dihydroxypropyl)amino]-2-nitrobenzene (HC Red No. 10), 5-chloro-1,4-[di(2,3-dihydroxypropyl)amino]-2-nitrobenzene (HC Red No. 11), 2-chloro-6-ethylamino-4-nitrophenol, 2-amino-6-chloro-4-nitrophenol, 4-[(2-hydroxyethyl)amino]-3-nitro-1-trifluoromethylbenzene (HC Yellow No. 13), 8-amino-2-bromo-5-hydroxy-4-imino-6-{[3-(trimethylammonio)-phenyl]amino}-1(4H)-naphthalenone chloride (C.I. 56059; Basic Blue No. 99), 1-[(4-aminophenyl)azo]-7-(trimethylammonio)-2-naphthol chloride (CI 12250; Basic Brown No. 16), 1-[(4-amino-2-nitrophenyl)azo]-7-(trimethylammonio)-2-naphthol chloride (Basic Brown No. 17), 2-hydroxy-1-[(2-methoxyphenyl)azo]-7-(trimethylammonio)naphthalene chloride (C.I. 12245; Basic Red No. 76), 3-methyl-1-phenyl-4-{[3-(trimethylammonio)phenyl]azo}pyrazol-5-one chloride (CA. 12719; Basic Yellow No. 57) and 2,6-diamino-3-[(pyridin-3-yl)azo]pyridine as well as the salts thereof. Particularly preferred among the aforesaid direct dyes are the following compounds, alone or in combination with one another: hydroxyethyl-2-nitro-p-toluidine, 2-hydroxyethylpicramic acid, 4-nitrophenylaminourea, tri(4-amino-3-methylphenyl)carbenium chloride (Basic Violet 2), 1,4-di-amino-9,10-anthracenedione (Disperse Violet 1), 1-(2-hydroxy-ethyl)amino-2-nitro-4-[di(2-hydro-xyethyl)amino]benzene (HC Blue No. 2), 4-[ethyl-(2-hydroxyethyl)amino]-1-[(2-hydroxyethyl)amino]-2-nitrobenzene hydrochloride (HC Blue No. 12), 1-amino-4-[di(2-hydroxyethyl)amino]-2-nitrobenzene hydrochloride (HC Red No. 13), 4-amino-1-[(2-hydroxyethyl)amino]-2-nitrobenzene (HC Red No. 3), 4-amino-3-nitrophenol, 4-[(2-hydroxyethyl)amino]-3-nitrophenol, 1-amino-5-chloro-4-[(2,3-dihydroxypropyl)amino]-2-nitrobenzene (HC Red No. 10), 5-chloro-1,4-[di(2,3-dihydroxypropyl)-amino]-2-nitrobenzene (HC Red No. 11), 2-chloro-6-ethylamino-4-nitrophenol, 2-amino-6-chloro-4-nitrophenol, 4-[(2-hydroxyethyl)amino]-3-nitro-1-trifluoromethylbenzene (HC Yellow No. 13), 8-amino-2-bromo-5-hydroxy-4-imino-6-{[3-(trimethylammonio)-phenyl]amino}-1(4H)-naphthalenone chloride (CI 56059; Basic Blue No. 99), 1-[(4-aminophenyl)azo]-7-(trimethylammonio)-2-naphthol chloride (C.I. 12250; Basic Brown No. 16), 1-[(4-amino-2-nitrophenyl)azo]-7-(trimethylammonio)-2-naphthol chloride (Basic Brown No. 17), 2-hydroxy-1-[(2-methoxyphenyl)azo]-7-(trimethylammonio)naphthalene chloride (C.I. 12245; Basic Red No. 76), 3-methyl-1-phenyl-4-{[3-(trimethylammonio)phenyl]azo}pyrazol-5-one chloride (C.I. 12719; Basic Yellow No. 57) and 2,6-diamino-3-[(pyridin-3-yl)azo]pyridine as well as the salts thereof.

In certain embodiments, the cosmetic composition as otherwise described herein comprises from 0.1 wt % to 15 wt %, preferably from 0.1 wt % to 10 wt %, even more preferably from 0.5 wt % to 8.0 wt % of at least one direct dye.

In certain embodiments, the cosmetic composition as otherwise described herein comprises a perfume or fragrance ingredient. Individual fragrance compounds, e.g. the synthetic products of the type of esters, ethers, aldehydes, ketones, alcohols and hydrocarbons can be used as fragrance or perfume oils. Fragrance compounds of the ester type are e.g. benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl-methylphenyl glycinate, allylcyclohexyl propionate, styrallyl propionate and benzyl salicylate. The ethers include for example benzyl ethyl ether, the aldehydes include e.g. the linear alkanals with 8 to 18 carbon atoms, citral, citronellal, citronellyloxyacetaldehyde, cyclamen aldehyde, hydroxycitronellal, lilial and bourgeonal, the ketones include e.g. the ionones, alpha-isomethylionone and methyl-cedryl ketone, the alcohols include anethole, citronellol, eugenol, geraniol, linalool, phenylethyl alcohol and terpineol, and the hydrocarbons include mainly the terpenes and balsams. Preferably, mixtures of various fragrances are used, which together produce an attractive perfume note. Perfume oils can also contain mixtures of natural odoriferous substances that can be obtained from vegetable or animal sources, e.g. pine oil, citrus oil, jasmine oil, lily oil, rose oil, or ylang-ylang oil. Essential oils of lower volatility, which are used mostly as flavor components, are also suitable as perfume oils, e.g. sage oil, chamomile oil, clove oil, melissa oil, mint oil, cinnamon leaf oil, linden flower oil, juniper berry oil, vetiver oil, olibanum oil, galbanum oil and labdanum oil. In certain embodiments, the cosmetic composition as otherwise described herein comprises from 0.01 wt % to 3.0 wt %, preferably from 0.05 wt % to 2.0 wt %, even more preferably from 0.1 wt % to 1.0 wt % of at least one perfume or fragrance ingredient.

In certain embodiments, the cosmetic composition as otherwise described herein comprises a carrier, solvent or diluent. In certain embodiments, the cosmetic composition as otherwise described herein comprises a solvent, wherein the solvent comprises water and/or alcohol. Solvent is useful for providing the compounds used in present invention in liquid form. In certain embodiments, the solvent is cosmetically acceptable. In certain embodiments, the cosmetic composition as otherwise described herein comprises at least 10 wt % water. Water is useful for economic reasons but also because it is cosmetically acceptable. Optionally the composition comprises water-miscible or water-soluble solvents such as lower alkyl alcohols. In certain embodiments, the cosmetic composition as otherwise described herein comprises C₁-C₅ alkyl monohydric alcohols, preferably C₂-C₃ alkyl alcohols. The alcohols which may be present are in particular lower monohydric or polyhydric alcohols having 1 to 4 carbon atoms customarily used for cosmetic purposes, such as preferably ethanol and isopropanol. Optionally, the composition comprises a water-soluble polyhydric alcohol. In certain embodiments, the water-soluble polyhydric alcohols are polyhydric alcohols having two or more hydroxyl groups in the molecule. In certain embodiments, the water-soluble polyhydric alcohol is selected from the group consisting of: dihydric alcohols such as ethylene glycol, propylene glycol, trimethylene glycol, 1,2-butylene glycol, 1,3-butylene glycol, 1,4-butylene glycol, tetramethylene glycol, 2,3-butylene glycol, pentamethylene glycol, 2-butene-1,4-diol, hexylene glycol, octylene glycol; trihydric alcohols such as glycerine, trimethylol propane, 1,2,6-hexanetriol and the like; tetrahydric alcohols such as penthaerythritol; pentahydric alcohols such as xylytol, etc.; hexahydric alcohols such as sorbitol, mannitol; polyhydric alcohol polymers such as diethylene glycol, dipropylene glycol, polyethylene glycol, polypropylene glycol, tetraethylene glycol, diglycerine, polyethylene glycol, triglycerine, tetraglycerine, polyglycerine; dihydric alcohol alkyl ethers such as ethylene glycol monomethyl ether, ethylene glycol monoethyl ether, ethylene glycol monobutyl ether, ethylene glycol monophenyl ether, ethylene glycol monohexyl ether, ethylene glycol mono-2-methylhexyl ether, ethylene glycol isoamyl ether, ethylene glycol benzyl ether, ethylene glycol isopropyl ether, ethylene glycol dimethyl ether, ethylene glycol diethyl ether, ethylene glycol dibutyl ether; dihydric alcohol alkyl ethers such as diethylene glycol monomethyl ether, diethylene glycol monoethyl ether, diethylene glycol monobutyl ether, diethylene glycol dimethyl ether, diethylene glycol diethyl ether, diethylene glycol butyl ether, diethylene glycol methyl ethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether; dihydric alcohol ether esters such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disuccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate; glycerine monoalkyl ethers such as xyl alcohol, selachyl alcohol, batyl alcohol; sugar alcohols such as sorbitol, maltitol, maltotriose, mannitol, sucrose, erythritol, glucose, fructose, starch sugar, maltose, xylytose, starch sugar reduced alcohol, glysolid, tetrahydrofurfuryl alcohol, POE tetrahydrofurfuryl alcohol, POP butyl ether, POP POE butyl ether, tripolyoxypropylene glycerine ether, POP glycerine ether, POP glycerine ether phosphoric acid, POP POE pentanerythritol ether, and mixtures thereof. In a preferred embodiment, the composition comprises a solvent selected from the group consisting of water, glycols, ethanol, and combinations thereof. In a preferred embodiment, the composition comprises an aqueous, alcoholic or aqueous-alcoholic solvent, and wherein the aqueous, alcoholic or aqueous-alcoholic solvent comprises water, ethanol, propanol, isopropanol, 1,2-propylene glycol, 1,3-propylene glycol, isobutanol, butanol, butyl glycol, butyl diglycol, glycerol, or a mixture thereof; preferably wherein the aqueous, alcoholic or aqueous-alcoholic solvent comprises water, ethanol, propanol, isopropanol, 1,2-propylene glycol, 1,3-propylene glycol, glycerol, or mixtures thereof; more preferably wherein the aqueous, alcoholic or aqueous-alcoholic solvent comprises water, isopropanol, 1,2-propylene glycol, 1,3-propylene glycol, or mixtures thereof; even more preferably wherein the aqueous, alcoholic or aqueous-alcoholic solvent consists of water or consists of a mixture of water and an alcohol wherein the alcohol is selected from the group consisting of isopropanol, 1,2-propylene glycol and 1,3-propylene glycol. Natural solvents can also be used. In certain embodiments, the cosmetic composition as otherwise described herein comprises a solvent selected from the group consisting of plant oil, honey, plant-derived sugar compositions, and mixtures thereof. In certain embodiments, the cosmetic composition as otherwise described herein comprises from 0.5 wt % to 90 wt %, preferably from 1.0 wt % to 80 wt %, even more preferably from 5.0 wt % to 70 wt % of at least one carrier, solvent and/or diluent.

In certain embodiments, the cosmetic composition as otherwise described herein comprises a propellant. In certain embodiments, the propellant is selected from compressed gas propellants and liquefied gas propellants. In certain embodiments, the compressed gas propellants are selected from the group consisting of air, nitrogen (N2), nitrous oxide (N₂O), carbon dioxide (CO₂), and mixtures thereof; preferably air, nitrogen (N2), and mixtures thereof; most preferably nitrogen (N2). In certain embodiments, the liquefied gas propellants are selected from the group consisting of dimethylether (DME), 1,1-difluoroethane (HFC-152a), 1,1,1,2-tetrafluoroethane (HFC-134a), pentane, n-butane, iso-butane, propane, trans-1,3,3,3-tetrafluoropropene (HF0-1234ze), and mixtures thereof, preferably dimethylether (DME), 1,1-difluoroethane (HFC-152a), and mixtures thereof. In a preferred embodiment, the propellant is selected from the group consisting of nitrogen, carbon dioxide, pentane, n-butane, iso-butane, propane, and combinations thereof. In certain embodiments, the cosmetic composition as otherwise described herein comprises from 0.5 wt % to 60 wt %, preferably from 1.0 wt % to 50 wt %, even more preferably from 2.0 wt % to 40 wt % of at least one propellant.

In certain embodiments, the cosmetic composition as otherwise described herein comprises a functional acid or an active ingredient. Functional acids and active ingredients are substances used to impart a clinical functionality to the skin or hair upon application. Functional acids and active ingredients are for example used as exfoliants, skin-brightening agents, self-tanning agents, anti-acne agents and anti-ageing agents. In another preferred embodiment of the invention, the compositions according to the invention contain one or more hydroxy acids, especially preferably one or more substances selected from alpha- and beta-hydroxy acids. The compositions according to the invention can contain, as hydroxy acids, preferably lactic acid, glycolic acid, salicylic acid and alkylated salicylic acids or citric acid. Furthermore, formulations according to the invention can contain other acidic components. Consideration may be given to the following as active ingredient: tartaric acid, mandelic acid, caffeic acid, pyruvic acid, oligo-oxa mono- and dicarboxylic acids, fumaric acid, retinoic acid, sulfonic acids, benzoic acid, kojic acid, fruit acid, malic acid, gluconic acid, pyruvic acid, galacturonic acid, ribonic acid, hyaluronic acid, and all derivatives thereof, polyglycol diacids in free or partial neutralized form, vitamin C (ascorbic acid), vitamin C derivatives (e.g. sodium ascorbyl phosphate, magnesium ascorbyl phosphate and magnesium ascorbyl glucoside), dihydroxyacetone, minoxidil, proteolytic enzymes (e.g. fruit enzymes from papaya, pumpkin and pineapple such as papainase and bromelin ananase), caffeine, niacinamide and its derivatives, diethyl toluamide (DEET), or skin-whitening actives such as arbutin or glycyrrhetic acid and salts thereof, glutathione, cysteine, resveratrol, 4-butylresorcinol, or plant extracts like pancratium maritimum extract or mulberry extract. In a preferred embodiment, the functional acid and/or an active ingredient is selected from the group consisting of salicylic acid, kojic acid, hyaluronic acid, ascorbic acid, and all derivatives thereof, dihydroxyacetone, arbutin, and combinations thereof. In certain embodiments, the cosmetic composition as otherwise described herein comprises from 0.05 wt % to 15 wt %, preferably from 0.1 wt % to 10 wt %, even more preferably from 0.5 wt % to 5.0 wt % of at least one functional acid or/and an active ingredient.

In certain embodiments, the cosmetic composition as otherwise described herein comprises a deodorant or an anti-perspirants. In certain embodiments, the cosmetic composition as otherwise described herein comprises a deodorising agent. In certain embodiments, the deodorising agent is selected from the group consisting of allantoin, bisabolol, and combinations thereof. In certain embodiments, the cosmetic composition as otherwise described herein comprises from 0.001 wt % to 10 wt %, or from 0.01 wt % to 9.0 wt %, or from 0.05 wt % to 8.0 wt %, or from 0.1 wt % to 5.0 wt % of at least one deodorising agent. The composition may comprise an antiperspirant. As antiperspirant it is possible to use aluminium chloride, aluminum chloride hydroxide, aluminum chloride dihydroxide, aluminum chlorohydrex polyethylene glycol complex, magnesium zirconium complexes or aluminum zirconium chloride hydroxide, for example. In certain embodiments, the cosmetic composition as otherwise described herein comprises from 0.001 wt % to 10 wt %, or from 0.01 wt % to 9.0 wt %, or from 0.05 wt % to 8.0 wt %, or from 0.1 wt % to 5.0 wt % of at least one antiperspirant.

In certain embodiments, the cosmetic composition as otherwise described herein comprises at least one viscosity modifier or thickening and/or gelling agent. The desired viscosity and rheology profile of the compositions can be adjusted by adding further thickeners and gelling agents. The viscosity-modifying substance is preferably a thickening polymer. In certain embodiments, the thickening polymer selected from the group consisting of: copolymers of at least one first monomer type, which is chosen from acrylic acid and methacrylic acid, and at least one second monomer type, which is chosen from esters of acrylic acid and ethoxylated fatty alcohol, crosslinked polyacrylic acid, crosslinked copolymers of at least one first monomer type, which is chosen from acrylic acid and methacrylic acid, and at least one second monomer type, which is chosen from esters of acrylic acid with C₁₀- to C₃₀-alcohols; copolymers of at least one first monomer type, which is chosen from acrylic acid and methacrylic acid, and at least one second monomer type, which is chosen from esters of itaconic acid and ethoxylated fatty alcohol; copolymers of at least one first monomer type, which is chosen from acrylic acid and methacrylic acid, at least one second monomer type, which is chosen from esters of itaconic acid and ethoxylated C₁₀- to C₃₀-alcohol and a third monomer type, chosen from C₁- to C₄-aminoalkyl acrylates; copolymers of two or more monomers chosen from acrylic acid, methacrylic acid, acrylic esters and methacrylic esters; copolymers of vinylpyrrolidone and ammonium acryloyldimethyltaurate; copolymers of ammonium acryloyldimethyltaurate and monomers chosen from esters of methacrylic acid and ethoxylated fatty alcohols, hydroxyethylcellulose, hydroxypropylcellulose, hydroxypropylguar, glyceryl polyacrylate, glyceryl polymethacrylate, copolymers of at least one C₂-, C₃- or C₄-alkylene and styrene, polyurethanes, hydroxypropyl starch phosphate, polyacrylamide, copolymer of maleic anhydride and methyl vinyl ether crosslinked with decadiene, carob seed flour, gums such as guar gum, karaya gum, xanthan gum or dehydroxanthan gum, carrageenan, hydrolyzed corn starch; copolymers of polyethylene oxide, fatty alcohols and saturated methylenediphenyl diisocyanate (e.g. PEG-150/stearyl alcohol/SMDI copolymer), and mixtures thereof. In a preferred embodiment, the viscosity modifier or thickening and/or gelling agent is selected from the group consisting of carbomers, acrylates copolymers, xanthan gum, hydroxyethylcellulose, laureth-2, and combinations thereof. In certain embodiments, the cosmetic composition as otherwise described herein comprises from 0.01 wt % to 15 wt %, preferably from 0.1 wt % to 10 wt %, even more preferably from 0.5 wt % to 5.0 wt % of at least one viscosity modifier or thickening and/or gelling agent.

In certain embodiments, the cosmetic composition as otherwise described herein comprises an alkalizing agent or pH adjusting agent. In certain embodiments, ammonia or caustic soda is suitable, but water-soluble, physiologically tolerable salts of organic and inorganic bases can also be considered. Optionally, the pH adjusting agent is selected from ammonium hydrogen carbonate, ammonia, monoethanolamine, ammonium hydroxide, ammonium carbonate. In certain embodiments, the alkalizing agents is selected from the group consisting of 2-amino-2-methyl-1-propanol, 2-amino-2-methyl-1,3-propanediol, 2-amino-2-ethyl-1,3-propanediol, tris(hydroxyl-methyl)-aminomethane, 2-amino-1-butanole, tris-(2-hydroxypropyl)-amine, 2,2-iminobisethanol, lysine, iminourea (guanidine carbonate), tetrahydro-1,4-oxazine, 2-amino-5-guanidin-valeric acid, 2-aminoethansulfonic acid, diethanolamine, triethanolamine, N-methyl ethanolamine, isopropanolamine, diisopropanolamine, triisopropanolamine, glucamine, sodium hydroxide, potassium hydroxide, lithium hydroxide and magnesium oxide, and mixtures thereof. To establish an acidic pH value, and acid can be included. In certain embodiments, the cosmetic composition as otherwise described herein comprises an acid selected from the group consisting of hydrochloric acid, phosphoric acid, acetic acid, formic acid, sulfuric acid, hydrochloric acid, citric acid, ascorbic acid, and mixtures thereof. Citric acid is most preferred in that it has high consumer acceptance. In certain embodiments, the acidic pH is adjusted with a buffer such as a phosphate buffer, a TRIS buffer or a citric buffer. The buffers may be used alone or in combination with an acid. In a preferred embodiment, the alkalizing or pH adjusting agent is selected from the group consisting of triethanolamine, sodium hydroxide, lactic acid, citric acid, and combinations thereof. In certain embodiments, the cosmetic composition as otherwise described herein comprises from 0.001 wt % to 5.0 wt %, preferably from 0.01 wt % to 3.0 wt %, even more preferably from 0.1 wt % to 1.0 wt % of at least one alkalizing or pH adjusting agent.

In certain embodiments, the composition/formulation comprises an anti-oxidant. In certain embodiments, the anti-oxidant is selected from the group consisting of amino acids, peptides, sugars, imidazoles, carotinoids, carotenes, chlorogenic acid, lipoic acid, thiols, thiol glycosyl esters, thiol N-acetyl esters, thiol methyl esters, thiol ethyl esters, thiol propyl esters, thiol amyl esters, thiol butyl esters, thiol lauryl esters, thiol palmitoyl esters, thiol oleyl esters, thiol linoleyl esters, thiol cholesteryl esters, thiol glyceryl esters, dilaurylthiodipropionate, distearylthiodipropionate, thiodipropionic acid, metal chelators, hydroxy acids, fatty acids, folic acids, vitamin C, tocopherol, vitamin A, stilbenes, derivatives and combinations thereof. In certain embodiments, the anti-oxidant is selected from the group consisting of glycine, histidine, tyrosine, tryptophan, urocaninic acid, D,L-carnosine, D-carnosine, L-carnosine, beta-carotene, alpha-carotene, lycopene, dihydrolipoic acid, aurothioglucose, propylthiouracil, thioredoxine, glutathione, cysteine, cystine, cystamine, buthioninsulfoximine, homocysteinsulfoximine, buthioninsulfone, penta-, hexa-, heptathioninsulfoximine, hydroxyfatty acids, palmitic acid, phytinic acid, lactoferrin, citric acid, lactic acid, malic acid, humic acid, bile acid, bilirubin, biliverdin, EDTA, EGTA, linoleic acid, linolenic acid, oleic acid, butylhydroxyanisol, trihydroxybutyrophenone, ubichinon, ubichinol, ascorbylpalmitate, Mg-ascorbylphosphate, ascorbylacetate, vitamin E acetate, vitamin A palmitate, carnosine, mannose, ZnO, ZnSO₄, selenium methionine, stilbenes, superoxide dismutase, and combinations thereof. In certain embodiments, the antioxidant is selected from the group consisting of vitamin A, vitamin A derivatives, vitamin E, vitamin E derivatives, and combinations thereof. In certain embodiments, the cosmetic composition as otherwise described herein comprises from 0.001 wt % to 10 wt %, preferably from 0.05 wt % to 5.0 wt %, even more preferably from 0.1 wt % to 3.0 wt %, most preferably from 0.05 wt % to 1.0 wt % of at least one antioxidant.

In certain embodiments, the composition/formulation comprises a chelant. In certain embodiments, the chelant is selected from the group consisting of EDTA, caprylhydroxamic acid, oxalate derivatives, disodium hydroxyethyliminodiacetate, galacturonic acid and derivatives, glucuronic acid and derivatives, lauroyl ethylenediamine triacetic acid, methyl dihydroxybenzoate, trisodium ethylenediamine disuccinate, phytic acid, itaconic acid, propane tricarboxylic acid, citric acid and derivatives (e.g. diammonium citrate, bismuth citrate and acetyl trihexyl citrate 2,6-dicarboxy pyridine), phosphoric and phosphonic acid derivatives (e.g. diethylenetriamine pentamethylene phosphonic acid, disodium azacycloheptane diphosphonate, glycereth-26 phosphate, disodium pyrophosphate, disodium salicylphosphate, aminotrimethylene phosphonic acid, phosphonobutanetricarboxylic acid, potassium trisphosphonomethylamine oxidebeta-alanine diacetic acid or cyclohexanediamine tetraacetic acid). In certain embodiments, the chelant is selected from the group consisting EDTA, oxalate derivatives, disodium salicylphosphate, and combinations thereof. In certain embodiments, the cosmetic composition as otherwise described herein comprises from 0.01 wt % to 2.0 wt %, preferably from 0.05 wt % to 1.5 wt %, even more preferably from 0.1 wt % to 1.0 wt %, most preferably from 0.05 wt % to 1.0 wt % of at least one chelant.

In certain embodiments, the cosmetic composition as otherwise described herein comprises an astringent. In certain embodiments, the astringent is selected from the group consisting of magnesium oxide, aluminium oxide, titanium dioxide, zirconium dioxide, zinc oxide, oxide hydrates, aluminium oxide hydrate (boehmite) and hydroxide, chlorohydrates of calcium, magnesium, aluminium, titanium, zirconium or zinc. In certain embodiments, the cosmetic composition as otherwise described herein comprises from 0.001 wt % to 10 wt %, or from 0.01 wt % to 9.0 wt %, or from 0.05 wt % to 8.0 wt %, or from 0.1 wt % to 5.0 wt % of at least one astringent.

In certain embodiments, the cosmetic composition as otherwise described herein comprises a sun protection agent and/or UV filter. Suitable sun protection agents and UV filters are disclosed in WO2013017262A1 (published on 7 Feb. 2013), from page 32, line 11 to the end of page 33. The photoprotective substances include, in particular, all of the photoprotective substances specified in EP1084696A1, which is incorporated herein by reference. In another preferred embodiment of the invention, the compositions according to the invention contain one or more substances selected from inorganic and organic UV filters and especially preferably are in the form of sunscreen compositions.

The compositions according to the invention can contain microfine titanium dioxide, mica-titanium oxide, iron oxides, mica-iron oxide, zinc oxide, silicon oxides, ultramarine blue or chromium oxides as pigments/micropigments and as inorganic sunscreen filters or UV filters. The organic sunscreen filters or UV filters are preferably selected from 4-aminobenzoic acid, 3-(4′-trimethylammonium)-benzylidene-bornan-2-one-methylsulfate, camphor benzalkonium methosulfate, 3,3,5-trimethyl-cyclohexylsalicylate, 2-hydroxy-4-methoxybenzophenone, 2-phenylbenzimidazole-5-sulfonic acid and their potassium, sodium and triethanolamine salts, 3,3′-(1,4-phenylenedimethine)-bis(7,7-dimethyl-2-oxobicyclo[2.2.1]-heptane-1-methanesulfonic acid) and salts thereof, 1-(4-tert-butylphenyl)-3-(4-methoxyphenyl)propane-1,3-dione, 3-(4′-sulfo)-benzylidene-bornan-2-one and salts thereof, 2-cyano-3,3-diphenylacrylic acid-(2-ethylhexyl ester), polymers of N-[2(and 4)-(2-oxoborn-3-ylidenemethyl)benzyl]-acrylamide, 4-methoxy-cinnamic acid-2-ethylhexyl ester, ethoxylated ethyl-4-aminobenzoate, 4-methoxy-cinnamic acid isoamyl ester, 2,4,6-tris-[p-(2-ethylhexyloxycarbonyl)anilino]-1,3,5-triazine, 2-(2H-benzotriazol-2-yl)-4-methyl-6-(2-methyl-3-(1,3,3,3-tetramethyl-1-(trimethylsilyloxy)-disiloxanyl)-propyl)phenol, 4,4′-[(6-[4-((1,1-dimethylethyl)-aminocarbonyl)phenylamino]-1,3,5-triazin-2,4-yl)diimino]bis-(benzoic acid-2-ethylhexyl ester), benzophenone-3, benzophenone-4 (acid), 3-(4′-methylbenzylidene)-D,L-camphor, 3-benzylidene-camphor, salicylic acid-2-ethylhexyl ester, 4-dimethylaminobenzoic acid-2-ethylhexyl ester, hydroxy-4-methoxy-benzophenone-5-sulfonic acid (sulfisobenzonum) and the sodium salt, 4-isopropylbenzylsalicylate, N, N, N-trimethyl-4-(2-oxoborn-3-ylidenemethyl)anilium methyl sulfate, homosalate (INN), oxybenzone (INN), 2-phenylbenzimidazole-5-sulfonic acid and their sodium, potassium, and triethanolamine salts, octylmethoxycinnamic acid, isopentyl-4-methoxycinnamic acid, isoamyl-p-methoxycinnamic acid, 2,4,6-trianilino-(p-carbo-2′-ethylhexyl-1′-oxy)-1,3,5-triazine (octyl triazone) phenol, 2-2(2H-benzotriazol-2-yl)-4-methyl-6-(2-methyl-3-(1,3,3,3-tetramethyl-1-(trimethylsilyl)oxy)-disiloxanyl)propyl (drometrizole trisiloxane) benzoic acid, 4,4-((6-(((1,1-dimethylethyl)amino)carbonyl)phenyl)amino)-1,3,5-triazine-2,4-diyl)diimino)bis,bis(2-ethylhexyl)ester) benzoic acid, 4,4-((6-(((1,1-dimethylethyl)amino)-carbonyl)phenyl)amino)-1,3,5-triazine-2,4-diyl)diimino)bis,bis(2-ethylhexyl)ester), 3-(4′-methylbenzylidene)-D,L-camphor (4-methylbenzylidene camphor), benzylidene-camphor-sulfonic acid, octocrylene, polyacrylamidomethyl-benzylidene-camphor, 2-ethylhexyl salicylate (octyl salicylate), 4-dimethyl-aminobenzoic acid ethyl-2-hexyl ester (octyl dimethyl PABA), PEG-25 PABA, 2-hydroxy-4-methoxybenzophenone-5-sulfonic acid (benzophenone-5) and the Na salt, 2,2′-methylene-bis-6-(2H-benzotriazol-2-yl)-4-(tetramethylbutyl)-1,1,3,3-phenol, sodium salt of 2-2′-bis-(1,4-phenylene)1H-benzimidazole-4,6-disulfonic acid, (1,3,5)-triazine-2,4-bis((4-(2-ethylhexyloxy)-2-hydroxy)-phenyl)-6-(4-methoxyphenyl), 2-ethylhexyl-2-cyano-3,3-diphenyl-2-propenoate, glyceryl octanoate di-p-methoxycinnamic acid, p-amino-benzoic acid and esters thereof, 4-tert-butyl-4′-methoxydibenzoylmethane, 4-(2-β-glucopyranoxy)propoxy-2-hydroxybenzophenone, octyl salicylate, methyl-2,5-diisopropylcinnamic acid, cinoxate, dihydroxy-dimethoxybenzophenone, disodium salt of 2,2′-dihydroxy-4,4′-dimethoxy-5,5′-disulfobenzophenone, dihydroxybenzophenone, 1,3,4-dimethoxyphenyl-4,4-dimethyl-1,3-pentanedione, 2-ethylhexyl-dimethoxybenzylidene-dioxoimidazolidine propionate, methylene-bis-benzotriazolyl tetramethylbutylphenol, phenyldibenzimidazole tetrasulfonate, bis-ethylhexyloxyphenol-methoxyphenol-triazine, tetrahydroxybenzophenones, terephthalylidene-dicamphor-sulfonic acid, 2,4,6-tris[4,2-ethylhexyloxycarbonyl)anilino]-1,3,5-triazine, methyl-bis(trimethylsiloxy)silyl-isopentyl trimethoxycinnamic acid, amyl-p-dimethylaminobenzoate, amyl-p-dimethylaminobenzoate, 2-ethylhexyl-p-dimethylaminobenzoate, isopropyl-p-methoxycinnamic acid/diisopropylcinnamic acid ester, 2-ethylhexyl-p-methoxycinnamic acid, 2-hydroxy-4-methoxybenzophenone, 2-hydroxy-4-methoxybenzophenone-5-sulfo acid and the trihydrate, and 2-hydroxy-4-methoxybenzophenone-5-sulfonate sodium salt, phenyl-benzimidazole-sulfonic acid, p-aminobenzoic acid butyl ester, methyl-3-[2,4-bis(methylethyl)phenyl]-2-propenoate, 3-(4-hydroxy)-3-methoxyphenyl)-2-propenoic acid, 5-methyl-2-(1-methylethyl)cyclohexanol-2-aminobenzoate, diethylmalonylbenzylidene oxypropene dimethicone, 2,4,6-tris(biphenyl-4-yl)-1,3,5-triazin and tris(2-hydroxyethyl)ammonium 2-hydroxybenzoate. In a preferred embodiment, the sun protection agent and/or UV filter is selected from the group consisting of 2-ethylhexyl 4-methoxycinnamate, methyl methoxycinnamate, 2-ethylhexyl salicylate, 2-ethylhexyl-2-cyano-3,3-diphenyl-2-propenoate, 2-hydroxy-4-methoxybenzophenone-5-sulfonic acid, polyethoxylated p-aminobenzoates, and combinations thereof. In certain embodiments, the cosmetic composition as otherwise described herein comprises from 0.001 wt % to 30 wt %, preferably from 0.05 wt % to 20 wt %, even more preferably from 0.1 wt % to 10 wt %, most preferably from 0.05 wt % to 5.0 wt % of at least one sun protection agent and/or UV filter.

In certain embodiments, the cosmetic composition as otherwise described herein comprises a skin conditioning agent. Skin conditioning agents such as emollients, humectants and occlusive agents are ingredients which help to maintain the soft and smooth appearance of the skin or which help to improve the condition of dry or damaged skin. In certain embodiments, the skin conditioning agent is selected from the group consisting of oily substances (description see above), functional acids or active ingredients (description see above), fatty acid N-alkylpolyhydroxyalkyl amides, fatty acids, triglycerides, panthenol, allantoin, bisabolol, glycerol, sorbitol, urea and derivatives thereof, trehalose, erythrulose, pyrrolidone carboxylic acid (PCA) and its salts, polyglucuronic acid, gluconolactone, petrolatum, ubichinon-10 and ubiquinol. In a preferred embodiment, the skin conditioning agent is selected from the group consisting of urea, glycerine, pyrrolidone carboxylic acid (PCA) and its salts, panthenol, petrolatum, and combinations thereof. In certain embodiments, the cosmetic composition as otherwise described herein comprises from 0.001 wt % to 5.0 wt %, preferably from 0.05 wt % to 3.0 wt %, even more preferably from 0.1 wt % to 1.0 wt % of at least one skin conditioning agent.

In certain embodiments, the cosmetic composition as otherwise described herein comprises an anti-foaming agent. Antifoams are chemicals which reduce the tendency of finished products to generate foam on shaking or agitation. In certain embodiments, the anti-foaming agent is selected from the group consisting of alcohols (e.g. ethanol, isopropyl alcohol or propyl alcohol), alkoxylated alcohols (e.g. laureth-5 butyl ether), silicon oils and resins (e.g. dimethicone and its derivatives such as cetyl dimethicone, phenyl dimethicone, PEG/PPG-12/18 dimethicone and hydrogen trifluoropropyl dimethicone, trimethylsiloxysilicate/dimethicone crosspolymer or polysilicone-10) and hydrophobic silica derivatives (e.g. silica silylate). In a preferred embodiment, the anti-foaming agent is selected from the group consisting of ethanol, dimethicone, silica silylate, and combinations thereof. In certain embodiments, the cosmetic composition as otherwise described herein comprises from 0.01 wt % to 5.0 wt %, preferably from 0.1 wt % to 3.0 wt %, even more preferably from 0.5 wt % to 2.0 wt % of at least one anti-foaming agent.

In certain embodiments, the cosmetic composition as otherwise described herein comprises a flavouring agent. In certain embodiments, the flavouring agent is selected from the group consisting of 1-acetonaphthalene, 1-decen-3-ol, p-methylbenzaldehyde, p-propenylphenyl methyl ether, aspartame, benzaldehyde, bromocinnamal, calcium cyclohexylsulfamate, calcium o-benzolufimide, carvone, cinnamic aldehyde, 3,7-dimethyl-6-octenoic acid, fruit sugar, glucose, glucosyl stevioside, honey, 3-methyl-1-butanol, 4-hydroxy-3-methoxy-1-propenylbenzene, malt sugar, menthol, eucalyptol, thymol, potassium 6-methyl-1,2,2-oxathiazin-4(3H)-one 2,2′-dioxide, isodulcitol, saccharine, stevioside, 1′,4,6′-trichloro-galacto-sucrose, sorbitol, saccharose, sodium saccharin, methyl salicylate vanillaldehyde, xylite, xylose and plant extracts. In a preferred embodiment, the flavouring agent is selected from the group consisting of benzaldehyde, cinnamic aldehyde, fruit sugar, stevioside and its derivatives, saccharine, saccharose, vanillaldehyde, xylite, and combinations thereof. In certain embodiments, the cosmetic composition as otherwise described herein comprises from 0.001 wt % to 3.0 wt %, preferably from 0.01 wt % to 2.0 wt %, even more preferably from 0.05 wt % to 1.0 wt % of at least one flavouring agent.

In certain embodiments, the cosmetic composition as otherwise described herein comprises an electrolyte. In certain embodiments, the electrolyte is selected from the group consisting of salts preferably ammonium or metal salts, especially preferably halides, for example CaCl₂, MgCl₂, LiCl, KCl and NaCl, carbonates, hydrogen carbonates, phosphates, sulfates, nitrates, especially preferably sodium chloride, sodium fluoride, sodium monofluorophosphate, stannous fluoride, and/or organic salts, preferably ammonium or metal salts, especially preferably of glycolic acid, lactic acid, citric acid, tartaric acid, mandelic acid, salicylic acid, ascorbic acid, pyruvic acid, fumaric acid, retinoic acid, sulfonic acids, benzoic acid, kojic acid, fruit acid, malic acid, gluconic acid or galacturonic acid. These also include aluminum salts, preferably aluminum hydrochloride or aluminum-zirconium complex salts. In a preferred embodiment of the invention the compositions according to the invention therefore contain one or more substances selected from inorganic and organic salts. As electrolyte, the compositions according to the invention can also contain mixtures of various salts. In a preferred embodiment, the electrolyte is selected from the group consisting of sodium chloride, magnesium chloride, sodium citrate, sodium acetate, sodium hyaluronate, and combinations thereof. In certain embodiments, the cosmetic composition as otherwise described herein comprises from 0.001 wt % to 5.0 wt %, preferably from 0.05 wt % to 3.0 wt %, even more preferably from 0.1 wt % to 1.0 wt % of at least one electrolyte.

In certain embodiments, the cosmetic composition as otherwise described herein comprises an oxidizing or reducing agent. In certain embodiments, the oxidizing or reducing agent is selected from the group consisting of ammonium persulfate, calcium peroxide, hydrogen peroxide, hypochlorous acid, sodium hypochlorite, potassium monopersulfate, sodium carbonate peroxide, ammonium thioglycolate, cysteine, glutathione, hydroquinone, mercaptopropionic acid, superoxide dismutase, thioglycerin, thioglycolic acid, thiolactic acid, sodium sulfite, sodium thioglycolate, potassium thioglycolate, cysteine, the compositions according to the invention can also contain mixtures of various agents. In a preferred embodiment, the oxidizing or reducing agent is selected from the group consisting of hydrogen peroxide, sodium hypochlorite, superoxide dismutase, thioglycolic acid, sodium thioglycolate, potassium thioglycolate, cysteine, sodium carbonate peroxide, and combinations thereof. In certain embodiments, the cosmetic composition as otherwise described herein comprises from 0.001 wt % to 10 wt %, preferably from 0.05 wt % to 7.0 wt %, even more preferably from 0.1 wt % to 5.0 wt % of at least one oxidizing or reducing agent.

### Composition Properties

The cosmetic compositions of the disclosure as otherwise described herein, in certain embodiments, may have beneficial properties. For example, in certain embodiments, the cosmetic composition has a viscosity of from 0 cPs to 20,000 cPs. In certain embodiments, the cosmetic composition has a viscosity of from 0.1 cPs to 10,000 cPs, or from 1 cPs to 5,000 cPs, or from 5 cPs to 3,500 cPs. The viscosity measurement conditions are defined in the definitions section above. Viscosity may be important for anti-drip reasons. Dripping can be inconvenient for the user. Furthermore, more viscous compositions can be useful for measured dispensing. In certain embodiments, the cosmetic composition has a viscosity of from 0 cPs to 1,000 cPs. This viscosity range is advantageous when the composition is in the form of a facial cleanser in view of the need for distribution on skin and ability to rinse off.

In certain embodiments, the cosmetic composition as otherwise described herein further comprises a viscosity-modifying substance. The viscosity-modifying substance is preferably a thickening polymer. In certain embodiments, the thickening polymer selected from the group consisting of: copolymers of at least one first monomer type, which is chosen from acrylic acid and methacrylic acid, and at least one second monomer type, which is chosen from esters of acrylic acid and ethoxylated fatty alcohol; crosslinked polyacrylic acid; crosslinked copolymers of at least one first monomer type, which is chosen from acrylic acid and methacrylic acid, and at least one second monomer type, which is chosen from esters of acrylic acid with C₁₀- to C₃₀-alcohols; copolymers of at least one first monomer type, which is chosen from acrylic acid and methacrylic acid, and at least one second monomer type, which is chosen from esters of itaconic acid and ethoxylated fatty alcohol; copolymers of at least one first monomer type, which is chosen from acrylic acid and methacrylic acid, at least one second monomer type, which is chosen from esters of itaconic acid and ethoxylated C₁₀- to C₃₀-alcohol and a third monomer type, chosen from C₁- to C₄-aminoalkyl acrylates; copolymers of two or more monomers chosen from acrylic acid, methacrylic acid, acrylic esters and methacrylic esters; copolymers of vinylpyrrolidone and ammonium acryloyldimethyltaurate; copolymers of ammonium acryloyldimethyltaurate and monomers chosen from esters of methacrylic acid and ethoxylated fatty alcohols; hydroxyethylcellulose; hydroxypropylcellulose; hydroxypropylguar; glyceryl polyacrylate; glyceryl polymethacrylate; copolymers of at least one C₂-, C₃- or C₄-alkylene and styrene; polyurethanes; hydroxypropyl starch phosphate; polyacrylamide; copolymer of maleic anhydride and methyl vinyl ether crosslinked with decadiene; carob seed flour; guar gum; xanthan; dehydroxanthan; carrageenan; karaya gum; hydrolyzed corn starch; copolymers of polyethylene oxide, fatty alcohols and saturated methylenediphenyl diisocyanate (e.g. PEG-150/stearyl alcohol/SMDI copolymer); and mixtures thereof.

In certain embodiments, the cosmetic composition has a pH value in the range of from 2.0 to 12.0, or 3.0 to 9.0, or 4.5 to 7.5, or 2.0 to 7.0, or 3.0 to 7.0, or 4.5 to 7.0, or pH 4 to pH 6.5, or pH 4 to pH 6, or pH 4.5 to pH 6.5, or pH 4.5 to pH 6, or pH 5.3 to pH 5.7, or pH 5 to pH 6, or pH of about 5.5. By varying the pH value, a cosmetic composition can be made available that is suitable for different applications.

In certain embodiments, the cosmetic composition is in liquid form. In an alternative embodiment, the cosmetic composition is in solid form. Optionally, the cosmetic composition is in powdered or granulated form. This is advantageous in that it is not needed to ship the weight of liquid over long distances, which has economic and environmental benefits. A solid form can be achieved by spray drying the composition or the employment of a rotary evaporator. The composition can be converted into liquid form after it has been shipped e.g. by adding water. Clarity: Clear compositions are useful in view of increased consumer acceptance.

### Composition Types

The cosmetic composition of the disclosure as otherwise described herein includes various types depending on the end use. For example, in certain embodiments, the cosmetic composition is selected from the group consisting of shampoo, conditioner (e.g., leave-in or rinse out), body wash, facial cleanser, face mask, bubble bath, intimate wash, bath oil, cleansing milk, micellar water, make-up remover, cleansing wipes, hair mask, perfume, liquid soap, shaving soap, shaving foam, cleansing foam, day cream, anti-ageing cream, body milk, body lotion, in-shower body lotion (i.e., no rinse body lotion), body mousse, face serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm (bb) cream, eyeliner, night cream, eye brow gel, highlighter, lip stain, hand sanitizer, hair oil, nail varnish remover, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, body scrub, cellulite treatment, bar soap, cuticle cream, lip balm, hair treatment, eye shadow, bath additive, body mist, eau de toilette, mouthwash, toothpaste, lubricating gel, moisturizer, serum, toner, aqua sorbet, cream gel, styling mousse, dry shampoo, lip stick, lip gloss, hydro-alcoholic gel, body oil, shower milk, illuminator, lip crayon, hair spray, combing cream, and sunblock.

Preferably, the composition is selected from the group consisting of shampoo, conditioner (e.g., leave-in or rinse out), body wash, facial cleanser, face mask, bubble bath, cleansing milk, micellar water, make-up remover, cleansing wipes, hair mask, liquid soap, shaving soap, shaving foam, cleansing foam, day cream, anti-ageing cream, body milk, body lotion, in-shower body lotion body mousse, face serum, eye cream, sunscreen lotion, sun cream, face cream, after-shave lotion, pre-shaving cream, depilatory cream, skin-whitening gel, self-tanning cream, anti-acne gel, hair oil, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, body scrub, bar soap, hair treatment, mouthwash, toothpaste, moisturizer, serum, toner, aqua sorbet, cream gel, styling mousse, hydro-alcoholic gel, body oil, shower milk, hair spray, combing cream, and sunblock.

In certain embodiments, the cosmetic composition is for use on skin. In certain embodiments, the composition is for use on the face, the neck, the body and/or around the eye area. In certain embodiments, the composition is an emulsion or gel, preferably an oil-in-water (o/w), cream gel, hydro-alcoholic gel or hydrogel composition. In a preferred embodiment, the composition has a viscosity from 100 000 to 200 000 mPa·s, preferably from 1 000 to 100 000 mPa·s, even more preferably from 2 000 to 50 000 mPa·s and very preferably from 5 000 to 30 000 mPa·s (measured at 25° C., Brookfield RVT, T-C spindle at 20 revolutions per minute).

In certain embodiments, the composition is a body or face care composition such as face creams, neck creams, body lotions, body milks, face serums, blemish balm creams, hand creams, foot creams, body butters, lip creams, eye creams, after-sun lotions, make-up removing lotions or body mists, diaper creams or baby lotions. Optionally the body or face care composition comprises from 0.1 wt % to 15 wt %, preferably from 0.5 wt % to 10 wt %, even more preferably from 1.0 wt % to 5.0 wt % of at least one emulsifier, coemulsifier and/or solubilizer. Emulsifiers, coemulsifiers and/or solubilizers are listed above. In certain embodiments, the emulsifier, coemulsifier and/or solubilizer is selected from the group consisting of glyceryl stearate, cetearyl alcohol, polysorbate 20, stearic acid, cetearyl glucoside, PEG-40 hydrogenated castor oil, cetyl phosphate, steareth-2, ceteth-10 phosphate, trilaureth-4 phosphate, polyglyceryl-2 sesquiisostearate, cetyl PEG/PPG-10/1 dimethicone, and combinations thereof.

Optionally the body or face care composition comprises from 0.01 wt % to 40 wt %, preferably from 0.05 wt % to 30 wt %, even more preferably from 0.1 wt % to 20 wt % of at least one oily substance. Oily substances are listed above. In certain embodiments, the oily substance is selected from the group consisting of sweet almond oil, argan oil, caprylic/capric triglyceride, dimethicone, squalane, apricot kernel oil, coconut oil, jojoba oil, shea butter, mineral oil, isopropyl isostearate, dicaprylyl carbonate, isohexadecane, and combinations thereof.

Optionally the body or face care composition comprises from 0.01 wt % to 20 wt %, preferably from 0.05 wt % to 10 wt %, even more preferably from 0.1 wt % to 5.0 wt % of at least one wax. Waxes are listed above. In certain embodiments, the wax is selected from the group consisting of carnauba wax, beeswax, candelilla wax, synthetic wax, polyethylene, paraffin wax, microcrystalline wax, hydrogenated vegetable oil, hydrogenated castor oil, rice bran wax, cetyl dimethicone, and combinations thereof.

Optionally the body or face care composition comprises from 0.01 wt % to 15 wt %, preferably from 0.1 wt % to 10 wt %, even more preferably from 0.3 wt % to 5.0 wt % of at least one viscosity modifier or thickening and/or gelling agent. Viscosity modifiers or thickening and/or gelling agents are listed above. In certain embodiments, the viscosity modifier or thickening and/or gelling agent is selected from the group consisting of carbomers, acrylates copolymers, xanthan gum, hydroxyethylcellulose, polyamides, and combinations thereof.

Optionally the body or face care composition comprises from 0.001 wt % to 5.0 wt %, preferably from 0.05 wt % to 3.0 wt %, even more preferably from 0.1 wt % to 1.0 wt % of at least one skin conditioning agent. Skin conditioning agents are listed above. In certain embodiments, the skin conditioning agent is selected from the group consisting of urea, glycerine, pyrrolidone carboxylic acid (PCA) and its derivatives, panthenol, petrolatum, and combinations thereof.

Optionally the body or face care composition comprises from 0.001 wt % to 10 wt %, preferably from 0.05 wt % to 5.0 wt %, even more preferably from 0.1 wt % to 3.0 wt %, most preferably from 0.05 wt % to 1.0 wt % of at least one antioxidant. Antioxidants are listed above. In certain embodiments, the antioxidant is selected from the group consisting of vitamin A, vitamin A derivatives, vitamin E, vitamin E derivatives, BHT, and combinations thereof.

Optionally the body or face care composition comprises from 0.001 wt % to 5.0 wt %, preferably from 0.01 wt % to 3.0 wt %, even more preferably from 0.1 wt % to 2.0 wt % of at least one biogenic active substance. Biogenic active substances are listed above. In certain embodiments, the biogenic active substance is selected from the group consisting of aloe vera extract, collagen hydrolysates, bisabolol, vitamin C, vitamin E, allantoin, vitamin B5, tocopherol acetate, retinyl palmitate, and combinations thereof.

Optionally the body or face care composition comprises from 0.01 wt % to 4.0 wt %, preferably from 0.1 wt % to 3.0 wt %, even more preferably from 0.4 wt % to 1.0 wt % of at least one preservative or preservation boosting ingredient. Preservatives or preservation boosting ingredients are listed above. In certain embodiments, the preservative or preservation boosting ingredient is selected from the group consisting of methylparben, phenoxyethanol, DMDM hydantoin, ethylhexylglycerin, sodium benzoate, potassium sorbate, levulinic acid, p-anisic acid, sorbitan caprylate, and combinations thereof.

Optionally the body or face care composition comprises from 0.01 wt % to 3.0 wt %, preferably from 0.05 wt % to 2.0 wt %, even more preferably from 0.1 wt % to 1.0 wt % of at least one perfume or fragrance ingredient. Perfume or fragrance ingredients or are listed above.

Optionally the body or face care composition comprises from 0.01 wt % to 20 wt %, preferably from 0.1 wt % to 10 wt %, even more preferably from 0.5 wt % to 5.0 wt % of at least one further auxiliary. Auxiliaries are listed above.

In a preferred embodiment, the body or face care composition has a pH value from 2.0 to 10.0, preferably from 3.0 to 9.0, even more preferably from 4.0 to 8.0 and very preferably from 5.0 to 7.0.

In certain embodiments, the composition is a cleansing composition such as body washes, face washes, micellar waters or gels, body scrubs, face peeling, facial exfoliators, liquid soaps, bath additives, bubble baths, shower creams or milks, shower foams and face masks. Optionally the cleansing composition comprises from 0.5 wt % to 25 wt %, preferably from 1.0 wt % to 20 wt %, even more preferably from 2.0 wt % to 15 wt % of at least one surfactant. Surfactants are listed above. In certain embodiments, the surfactant is selected from the group consisting of sodium laureth sulfate, sodium lauryl sulfate, ammonium lauryl sulfate, sodium lauroyl sarcosinate, sodium methyl cocoyl taurate, cocamidopropyl betaine, sodium cocoyl glutamate, lauryl glucoside, cocoyl methyl glucamide, and combinations thereof.

Optionally the cleansing composition comprises from 0.01 wt % to 15 wt %, preferably from 0.1 wt % to 10 wt %, even more preferably from 0.5 wt % to 5.0 wt % of at least one viscosity modifier or thickening agent. Viscosity modifier or thickening agents are listed above. In certain embodiments, the viscosity modifier or thickening agent is selected from the group consisting of carbomers, acrylates copolymers, xanthan gum, laureth-3 or 4, cocamide DEA, coco glucosides, stearyl alcohol, and combinations thereof.

Optionally the cleansing composition comprises from 0.01 wt % to 15 wt %, preferably from 0.05 wt % to 10 wt %, even more preferably from 0.5 wt % to 5.0 wt % of at least one particulate substance. Particulate substances are listed above. In certain embodiments, the particulate substance is selected from the group consisting of silica, mica, bentonite, kaolin, talc, polyethylene, clay, and combinations thereof.

Optionally the cleansing composition comprises from 0.001 wt % to 30 wt %, preferably from 0.05 wt % to 20 wt %, even more preferably from 0.1 wt % to 10 wt % of at least one oily substance. Oily substances are listed above. In certain embodiments, the oily substance is selected from the group consisting of sweet almond oil, caprylic/capric triglycerides, dimethicone, mineral oil, squalane, castor oil, isopropyl isostearate, jojoba oil, dicaprylyl carbonate, isohexadecane, C₁₂-C₁₅ alkyl benzoate, and combinations thereof.

Optionally the cleansing composition comprises from 0.05 wt % to 15 wt %, preferably from 0.1 wt % to 10 wt %, even more preferably from 0.5 wt % to 5.0 wt % of at least one functional acid or/and an active ingredient. Functional acids or/and an active ingredients are listed above. In certain embodiments, the functional acid or/and an active ingredient is selected from the group consisting of alpha- and beta-hydroxy acids, lactic acid, glycolic acid, salicylic acid, citric acid, vitamin C derivatives, proteolytic enzymes, and combinations thereof.

Optionally the cleansing composition comprises from 0.001 wt % to 5.0 wt %, preferably from 0.05 wt % to 3.0 wt %, even more preferably from 0.1 wt % to 1.0 wt % of at least one electrolyte. Electrolytes are listed above. In certain embodiments, the electrolyte is selected from the group consisting of sodium chloride, magnesium chloride, sodium citrate, sodium acetate, and combinations thereof.

Optionally the cleansing composition comprises from 0.01 wt % to 5.0 wt %, preferably from 0.1 wt % to 3.0 wt %, even more preferably from 0.4 wt % to 1.0 wt % of at least one preservative or preservation boosting ingredient. Preservatives or preservation boosting ingredients are listed above. In certain embodiments, the preservative or preservation boosting ingredient is selected from the group consisting of sodium benzoate, methylisothiazolinone, benzoic acid, methylparaben, phenoxyethanol, DMDM hydantoin, potassium sorbate, and combinations thereof.

Optionally the cleansing composition comprises from 0.01 wt % to 20 wt %, preferably from 0.1 wt % to 10 wt %, even more preferably from 0.5 wt % to 5.0 wt % of at least one further auxiliary. Auxiliaries are listed above.

In a preferred embodiment, the cleansing composition has a pH value from 2.0 to 10.0, preferably from 3.0 to 8.0, even more preferably from 4.0 to 7.0.

In certain embodiments, the composition is a sun care composition such as sun sprays, sun milks, sun lotions, sun gels. Body and face care compositions with sun protection agents and/or UV filters such as day creams, hand creams, foundations, lip balms and face serums can also serve as sun care compositions. Optionally the sun care composition comprises from 0.1 wt % to 15 wt %, preferably from 0.5 wt % to 10 wt %, even more preferably from 1.0 wt % to 5.0 wt % of at least one emulsifier, coemulsifier and/or solubilizer. Emulsifiers, coemulsifiers and/or solubilizers are listed above. In certain embodiments, the emulsifier, coemulsifier and/or solubilizer is selected from the group consisting of glyceryl stearate, cetearyl alcohol, polysorbate 20, stearic acid, cetearyl glucoside, PEG-40 hydrogenated castor oil, cetyl phosphate, steareth-2, ceteth-10 phosphate, trilaureth-4 phosphate, polyglyceryl-2 sesquiisostearate, cetyl PEG/PPG-10/1 dimethicone, and combinations thereof.

Optionally the sun care composition comprises from 0.001 wt % to 50 wt %, preferably from 0.05 wt % to 40 wt %, even more preferably from 0.1 wt % to 30 wt % of at least one oily substance. Oily substances are listed above. In certain embodiments, the oily substance is selected from the group consisting of sweet almond oil, caprylic/capric triglycerides, dimethicone, mineral oil, squalane, castor oil, isopropyl isostearate, jojoba oil, dicaprylyl carbonate, cyclopentasiloxane, isohexadecane, C₁₂-C₁₅ alkyl benzoate, and combinations thereof.

Optionally the sun care composition comprises from 0.001 wt % to 30 wt %, preferably from 0.05 wt % to 20 wt %, even more preferably from 0.1 wt % to 10 wt %, most preferably from 0.05 wt % to 5.0 wt % of at least one sun protection agent and/or UV filter. Sun protection agents and/or UV filters are listed above. In certain embodiments, the sun protection agent and/or UV filter is selected from the group consisting of 2-ethylhexyl 4-methoxycinnamate, methyl methoxycinnamate, 2-ethylhexyl salicylate, 2-ethylhexyl-2-cyano-3,3-diphenyl-2-propenoate, 2-hydroxy-4-methoxybenzophenone-5-sulfonic acid, polyethoxylated p-aminobenzoates, and combinations thereof.

Optionally the sun care composition comprises from 0.1 wt % to 10 wt %, preferably from 0.5 wt % to 7.5 wt %, even more preferably from 1.0 wt % to 5.0 wt % of at least one film former. Film formers are listed above. In certain embodiments, the film former is selected from the group consisting of VP/eicosene copolymer, acrylates/octylacrylamide copolymer, VP/VA copolymer, styrene/acrylates copolymer, acrylates copolymer, butyl ester of PVM/MA copolymer, hydroxyethylcellulose, polypropylsilsesquioxane, polyurethane-64, acrylates/polytrimethylsiloxymethacrylate copolymer, and combinations thereof.

Optionally the sun care composition comprises from 0.01 wt % to 5.0 wt %, preferably from 0.1 wt % to 3.0 wt %, even more preferably from 0.4 wt % to 1.0 wt % of at least one preservative or preservation boosting ingredient. Preservatives or preservation boosting ingredients are listed above. In certain embodiments, the preservative or preservation boosting ingredient is selected from the group consisting of methylparben, phenoxyethanol, DMDM hydantoin, ethylhexylglycerin, sodium benzoate, potassium sorbate, sorbitan caprylate, and combinations thereof.

Optionally the sun care composition comprises from 0.01 wt % to 20 wt %, preferably from 0.1 wt % to 10 wt %, even more preferably from 0.5 wt % to 5.0 wt % of at least one further auxiliary. Auxiliaries are listed above. In a preferred embodiment, the sun care composition has a pH value from 3.0 to 9.0, preferably from 4.0 to 8.0, even more preferably from 5.0 to 7.0.

In certain embodiments, the composition is a face toner. Optionally the face toner composition comprises from 0.001 wt % to 5.0 wt %, preferably from 0.05 wt % to 3.0 wt %, even more preferably from 0.1 wt % to 1.0 wt % of at least one skin conditioning agent. Skin conditioning agents are listed above. In certain embodiments, the skin conditioning agent is selected from the group consisting of glycerin, urea, hydroxyethyl urea, allantoin, bisabolol, panthenol, and combinations thereof.

Optionally the face toner composition comprises from 0.01 wt % to 15 wt %, preferably from 0.1 wt % to 10 wt %, even more preferably from 0.5 wt % to 5.0 wt % of at least one viscosity modifier or thickening agent. Viscosity modifier or thickening agents are listed above. In certain embodiments, the viscosity modifier or thickening agent is selected from the group consisting of carbomers, acrylates copolymers, xanthan gum, carrageenan, and combinations thereof.

Optionally the face toner composition comprises from 0.01 wt % to 5.0 wt %, preferably from 0.1 wt % to 3.0 wt %, even more preferably from 0.4 wt % to 1.0 wt % of at least one preservative or preservation boosting ingredient. Preservatives or preservation boosting ingredients are listed above. In certain embodiments, the preservative or preservation boosting ingredient is selected from the group consisting of methylparaben, phenoxyethanol, methylisothiazolinone, ethylhexylglycerin, and combinations thereof.

Optionally the face toner composition comprises from 0.01 wt % to 20 wt %, preferably from 0.1 wt % to 10.0 wt %, even more preferably from 0.5 wt % to 5.0 wt % of at least one further auxiliary. Auxiliaries are listed above.

In a preferred embodiment, the face toner composition has a pH value from 3.0 to 9.0, preferably from 4.0 to 8.0, even more preferably from 5.0 to 7.0.

In certain embodiments, the composition is a bar soap or syndet composition. Optionally the bar soap or syndet composition comprises from 1.0 wt % to 50 wt %, preferably from 2.0 wt % to 30 wt %, even more preferably from 5.0 wt % to 20 wt % of at least one surfactant. Surfactants are listed above. In certain embodiments, the surfactant is selected from the group consisting of disodium lauryl sulfosuccinate, sodium palm kernelate, sodium palmate, sodium cocoate, sodium tallowate, palm kernel acid, sodium cocoyl isethionate, sodium isethionate, sodium lauryl sulfate, cocamidopropyl betaine, and combinations thereof.

Optionally the bar soap or syndet composition comprises from 0.01 wt % to 20 wt %, preferably from 0.05 wt % to 15 wt %, even more preferably from 0.5 wt % to 10 wt % of at least one particulate substance. Particulate substances are listed above. In certain embodiments, the particulate substance is selected from the group consisting of silica, calcium carbonate, sodium bicarbonate, titanium dioxide, polyethylene, and combinations thereof.

Optionally the bar soap or syndet composition comprises from 0.001 wt % to 5.0 wt %, preferably from 0.05 wt % to 3.0 wt %, even more preferably from 0.1 wt % to 1.0 wt % of at least one electrolyte. Electrolytes are listed above. In certain embodiments, the electrolyte is selected from the group consisting of sodium chloride, magnesium chloride, sodium citrate, sodium acetate, and combinations thereof.

Optionally the bar soap or syndet composition comprises from 0.01 wt % to 20 wt %, preferably from 0.1 wt % to 10 wt %, even more preferably from 0.5 wt % to 5.0 wt % of at least one further auxiliary. Auxiliaries are listed above.

In a preferred embodiment, the bar soap or syndet composition has a pH value from 3.0 to 13.0, preferably from 4.0 to 12.0, even more preferably from 5.0 to 10.0.

In certain embodiments, the composition is a composition suitable for wet wipes. Optionally the composition suitable for wet wipes comprises from 0.1 wt % to 10 wt %, preferably from 0.3 wt % to 5.0 wt %, even more preferably from 0.5 wt % to 3.0 wt % of at least one emulsifier, coemulsifier and/or solubilizer. Emulsifiers, coemulsifiers and/or solubilizers are listed above. In certain embodiments, the emulsifier, coemulsifier and/or solubilizer is selected from the group consisting of glyceryl stearate, cetearyl alcohol, polysorbate 20, stearic acid, cetearyl glucoside, PEG-40 hydrogenated castor oil, and combinations thereof.

Optionally the composition suitable for wet wipes comprises from 0.001 wt % to 30 wt %, preferably from 0.05 wt % to 20 wt %, even more preferably from 0.1 wt % to 10 wt % of at least one oily substance. Oily substances are listed above. In certain embodiments, the oily substance is selected from the group consisting of caprylic/capric triglycerides, dimethicone, mineral oil, squalane, castor oil, isopropyl isostearate, jojoba oil, isohexadecane, C₁₂-C₁₅ alkyl benzoate, and combinations thereof.

Optionally the composition suitable for wet wipes comprises from 0.01 wt % to 5.0 wt %, preferably from 0.1 wt % to 3.0 wt %, even more preferably from 0.4 wt % to 1.0 wt % of at least one preservative or preservation boosting ingredient. Preservatives or preservation boosting ingredients are listed above. In certain embodiments, the preservative or preservation boosting ingredient is selected from the group consisting of methylparaben, phenoxyethanol, sodium benzoate, potassium sorbate, benzoic acid, ethylhexylglycerin, sorbitan caprylate, and combinations thereof.

Optionally composition suitable for wet wipes comprises from 0.01 wt % to 20 wt %, preferably from 0.1 wt % to 10 wt %, even more preferably from 0.5 wt % to 5.0 wt % of at least one further auxiliary. Auxiliaries are listed above.

In a preferred embodiment, the composition suitable for wet wipes has a pH value from 2.0 to 10, preferably from 3.0 to 9.0, even more preferably from 4.0 to 8.0.

In certain embodiments, the composition is a deodorizing and/or antiperspirant composition. In certain embodiments, the composition is in the form of a cream, a roll-on, a solid, an aerosol or a gel. Optionally the deodorizing and/or antiperspirant composition comprises from 0.001 wt % to 10 wt %, or from 0.01 wt % to 9.0 wt %, or from 0.05 wt % to 8.0 wt %, or from 0.1 wt % to 5.0 wt % of at least one antiperspirant and/or deodorizing agent. Antiperspirants and deodorizing agents are listed above. In certain embodiments, the antiperspirant and/or deodorizing agent is selected from the group consisting of aluminium chlorohydrate, allantoin, bisabolol, aluminium chloride, magnesium zirconium complexes or aluminum zirconium chloride hydroxide, and combinations thereof.

Optionally the deodorizing and/or antiperspirant composition comprises 0.01 wt % to 3.0 wt %, preferably from 0.05 wt % to 2.0 wt %, even more preferably from 0.1 wt % to 1.0 wt % of at least one perfume or fragrance ingredient. Perfume or fragrance ingredients or are listed above. In certain embodiments, the perfume or fragrance ingredient is selected from the group consisting of linalool, limonene, geraniol, coumarin, butylphenyl methylpropional, alpha-isomethyl ionone, citral, hexyl cinnamal, and combinations thereof.

Optionally the deodorizing and/or antiperspirant composition comprises from 0.5 wt % to 60 wt %, preferably from 1.0 wt % to 50 wt %, even more preferably from 2.0 wt % to 40 wt % of at least one propellant. Propellants are listed above. In certain embodiments, the propellant is selected from the group consisting of nitrogen, carbon dioxide, pentane, n-butane, iso-butane, propane, and combinations thereof.

Optionally the deodorizing and/or antiperspirant composition comprises from 0.01 wt % to 20 wt %, preferably from 0.1 wt % to 10 wt %, even more preferably from 0.5 wt % to 5.0 wt % of at least one further auxiliary. Auxiliaries are listed above.

In a preferred embodiment, the deodorizing and/or antiperspirant composition has a pH value from 2.0 to 8.0, preferably from 3.0 to 7.0, even more preferably from 4.0 to 6.0.

In certain embodiments, the composition is a shaving composition. Optionally the shaving composition comprises from 0.1 wt % to 10 wt %, preferably from 0.3 wt % to 5.0 wt %, even more preferably from 0.5 wt % to 3.0 wt % of at least one emulsifier, coemulsifier and/or solubilizer. Emulsifiers, coemulsifiers and/or solubilizers are listed above. In certain embodiments, the emulsifier, coemulsifier and/or solubilizer is selected from the group consisting of cetearyl alcohol, polysorbate 20, palmitic acid, laureth-23, stearic acid, cetearyl glucoside, PEG-40 hydrogenated castor oil, and combinations thereof.

Optionally the shaving composition comprises from 0.001 wt % to 5.0 wt %, preferably from 0.05 wt % to 3.0 wt %, even more preferably from 0.1 wt % to 1.0 wt % of at least one skin conditioning agent. Skin conditioning agents are listed above. In certain embodiments, the skin conditioning agent is selected from the group consisting of glycerin, urea, sorbitol, aloe vera leaf juice, and combinations thereof

Optionally the shaving composition comprises from 0.5 wt % to 60 wt %, preferably from 1.0 wt % to 50 wt %, even more preferably from 2.0 wt % to 40 wt % of at least one propellant. Propellants are listed above. In certain embodiments, the propellant is selected from the group consisting of nitrogen, carbon dioxide, pentane, n-butane, iso-butane, propane, and combinations thereof.

Optionally the shaving composition comprises from 0.1 wt % to 20 wt %, preferably from 0.3 wt % to 10 wt %, even more preferably from 0.5 wt % to 5.0 wt % of at least one surfactant. Surfactants are listed above. In certain embodiments, the surfactant is selected from the group consisting of sodium laureth sulfate, sodium lauryl sulfate, ammonium lauryl sulfate, sodium lauroyl sarcosinate, cocamidopropyl betaine, and combinations thereof.

Optionally the shaving composition comprises from 0.01 wt % to 20 wt %, preferably from 0.1 wt % to 10 wt %, even more preferably from 0.5 wt % to 5.0 wt % of at least one further auxiliary. Auxiliaries are listed above.

In certain embodiments, the cosmetic, dermatological or pharmaceutical composition is for use on hair and/or scalp. In certain embodiments, the composition is an emulsion or gel, preferably an oil-in-water (o/w), cream gel, hydro-alcoholic gel or hydrogel composition. In a preferred embodiment, the hair care composition has a viscosity from 100 000 to 150 000 mPa·s, preferably from 1 000 to 100 000 mPa·s, more preferably from 2 000 to 50 000 mPa·s and very preferably from 5 000 to 30 000 mPa·s (25° C., Brookfield RVT, T-C spindle at 20 revolutions per minute).

In certain embodiments, the composition is a shampoo composition. Optionally the shampoo composition comprises from 0.5 wt % to 30 wt %, preferably from 1.0 wt % to 15 wt %, even more preferably from 2.0 wt % to 10 wt % of at least one surfactant. Surfactants are listed above. In certain embodiments, the surfactant is selected from the group consisting of sodium laureth sulfate, sodium lauryl sulfate, ammonium lauryl sulfate, sodium lauroyl sarcosinate, sodium methyl cocoyl taurate, cocamidopropyl betaine, sodium cocoyl glutamate, lauryl glucoside, cocoyl methyl glucamide, and combinations thereof.

Optionally the shampoo composition comprises from 0.1 wt % to 10 wt %, preferably from 0.5 wt % to 7.5 wt %, even more preferably from 1.0 wt % to 5.0 wt % of at least one cationic polymer. Cationic polymers are listed above. In certain embodiments, the cationic polymer is selected from the group consisting of polyquaternium-10, guar hydroxypropyltrimonium chloride, polyquaternium-7, polyquaternium-6, and combinations thereof.

Optionally the shampoo composition comprises from 0.1 wt % to 15 wt %, preferably from 0.5 wt % to 10 wt %, even more preferably from 1.0 wt % to 5.0 wt % of at least one pearlizing agent. Pearlizing agents are listed above. In certain embodiments, the cationic polymer is selected from the group consisting of ethylene glycol distearates and/or polyethylene glycol distearates with 3 glycol units on average, and combinations thereof.

Optionally the shampoo composition comprises from 0.001 wt % to 5.0 wt %, preferably from 0.05 wt % to 3.0 wt %, even more preferably from 0.1 wt % to 1.0 wt % of at least one electrolyte. Electrolytes are listed above. In certain embodiments, the electrolyte is selected from the group consisting of sodium chloride, magnesium chloride, sodium citrate, sodium acetate, and combinations thereof.

Optionally the shampoo composition comprises from 0.01 wt % to 15 wt %, preferably from 0.1 wt % to 10 wt %, even more preferably from 0.5 wt % to 5.0 wt % of at least one viscosity modifier or thickening agent. Viscosity modifier or thickening agents are listed above. In certain embodiments, the viscosity modifier or thickening agent is selected from the group consisting of carbomers, acrylates copolymers, xanthan gum, laureth-3 or 4, cocamide DEA, coco glucosides, stearyl alcohol, and combinations thereof.

Optionally the shampoo composition comprises from 0.01 wt % to 5.0 wt %, preferably from 0.1 wt % to 3.0 wt %, even more preferably from 0.4 wt % to 1.0 wt % of at least one preservative or preservation boosting ingredient. Preservatives or preservation boosting ingredients are listed above.

Optionally the shampoo composition comprises from 0.01 wt % to 20 wt %, preferably from 0.1 wt % to 10 wt %, even more preferably from 0.5 wt % to 5.0 wt % of at least one further auxiliary. Auxiliaries are listed above.

In certain embodiments, the composition is a hair conditioning and/or hair and/or scalp treatment composition such as leave-in and rinse-off conditioners, masks, lotions, combing creams, detangling creams, anti-frizz liquids, hair serums, scalp serums, color protection creams.

Optionally the hair conditioning and/or hair and/or scalp treatment composition comprises from 0.1 wt % to 15 wt %, preferably from 0.5 wt % to 10 wt %, even more preferably from 1.0 wt % to 5.0 wt % of at least one emulsifier, coemulsifier and/or solubilizer. Emulsifiers, coemulsifiers and/or solubilizers are listed above. In certain embodiments, the emulsifier, coemulsifier and/or solubilizer is selected from the group consisting of cetearyl alcohol, cetrimonium chloride, behentrimonium chloride, steartrimonium chloride, cetyl alcohol, stearyl alcohol, stearic acid, isostearamidopropyl dimethylamine, and combinations thereof.

Optionally the hair conditioning and/or hair and/or scalp treatment composition comprises from 0.01 wt % to 20 wt %, preferably from 0.05 wt % to 10 wt %, even more preferably from 0.1 wt % to 5.0 wt % of at least one oily substance. Oily substances are listed above. In certain embodiments, the oily substance is selected from the group consisting of dimethicone, squalene, amodimethicone, argan oil, jojoba oil, cyclopentasiloxane, mineral oil, castor oil, shea butter, and combinations thereof.

Optionally the hair conditioning and/or hair and/or scalp treatment composition comprises from 0.1 wt % to 10 wt %, preferably from 0.5 wt % to 7.5 wt %, even more preferably from 1.0 wt % to 5.0 wt % of at least one cationic polymer. Cationic polymers are listed above. In certain embodiments, the cationic polymer is selected from the group consisting of polyquaternium-10, guar hydroxypropyltrimonium chloride, polyquaternium-7, polyquaternium-6, and combinations thereof.

Optionally the hair conditioning and/or hair and/or scalp treatment composition comprises from 0.001 wt % to 5.0 wt %, preferably from 0.01 wt % to 3.0 wt %, even more preferably from 0.1 wt % to 2.0 wt % of at least one biogenic active substance. Biogenic active substances are listed above. In certain embodiments, the biogenic active substance is selected from the group aloe collagen hydrolysates, bisabolol, allantoin, hydrolyzed wheat protein, hydrolyzed silk, hydrolyzed keratin, amino acids and its derivatives, glycoproteins, and combinations thereof.

Optionally the hair conditioning and/or hair and/or scalp treatment composition comprises from 0.01 wt % to 5.0 wt %, preferably from 0.1 wt % to 3.0 wt %, even more preferably from 0.4 wt % to 1.0 wt % of at least one preservative or preservation boosting ingredient. Preservatives or preservation boosting ingredients are listed above. In certain embodiments, the preservative or preservation boosting ingredient is selected from the group consisting of sodium benzoate, methylparaben, phenoxyethanol, methylisothiazolinone, DMDM hydantoin, methylchloroisothiazolinone, zinc pyrithione, and combinations thereof.

Optionally the hair conditioning and/or hair and/or scalp treatment composition comprises from 0.01 wt % to 20 wt %, preferably from 0.1 wt % to 10 wt %, even more preferably from 0.5 wt % to 5.0 wt % of at least one further auxiliary. Auxiliaries are listed above

In certain embodiments, the composition is a hair styling composition such as mousses, gels, sprays and waxes.

Optionally the hair styling composition comprises from 0.1 wt % to 10 wt %, preferably from 0.5 wt % to 7.5 wt %, even more preferably from 1.0 wt % to 5.0 wt % of at least one film former. Film formers are listed above. In certain embodiments, the film former is selected from the group consisting of PVP, VPNA copolymer, styrene/acrylates copolymer, acrylates copolymer, butyl ester of PVM/MA copolymer, hydroxyethylcellulose, chitosan, polyquaternium-10, polypropylsilsesquioxane, polyurethane-64, and combinations thereof.

Optionally the hair styling composition comprises from 0.1 wt % to 10 wt %, preferably from 0.5 wt % to 7.5 wt %, even more preferably from 1.0 wt % to 5.0 wt % of at least one cationic polymer. Cationic polymers are listed above. In certain embodiments, the cationic polymer is selected from the group consisting of polyquaternium-10, guar hydroxypropyltrimonium chloride, polyquaternium-7, polyquaternium-6, and combinations thereof.

Optionally the hair styling composition comprises from 0.5 wt % to 60 wt %, preferably from 1.0 wt % to 50 wt %, even more preferably from 2.0 wt % to 40 wt % of at least one propellant. Propellants are listed above. In certain embodiments, the propellant is selected from the group consisting of nitrogen, carbon dioxide, pentane, n-butane, iso-butane, propane, and combinations thereof.

Optionally the hair styling composition comprises from 0.01 wt % to 5.0 wt %, preferably from 0.1 wt % to 3.0 wt %, even more preferably from 0.4 wt % to 1.0 wt % of at least one preservative or preservation boosting ingredient. Preservatives or preservation boosting ingredients are listed above. In certain embodiments, the preservative or preservation boosting ingredient is selected from the group consisting of sodium benzoate, methylparaben, phenoxyethanol, methylisothiazolinone, DMDM hydantoin, methylchloroisothiazolinone, zinc pyrithione, and combinations thereof.

Optionally the hair styling composition comprises from 0.01 wt % to 20 wt %, preferably from 0.1 wt % to 10.0 wt %, even more preferably from 0.5 wt % to 5.0 wt % of at least one further auxiliary. Auxiliaries are listed above

In certain embodiments, the composition is a mouthwash composition. Optionally mouthwash composition comprises from 0.001 wt % to 3.0 wt %, preferably from 0.01 wt % to 2.0 wt %, even more preferably from 0.05 wt % to 1.0 wt % of at least one flavouring agent. Flavouring agents are listed above. In certain embodiments, the flavouring agent is selected from the group consisting of stevioside, xylite, sodium saccharin, sorbitol, methyl salicylate, menthol, eucalyptol, thymol, and combinations thereof.

Optionally the mouthwash composition comprises from from 0.1 wt % to 6.0 wt %, preferably from 0.2 wt % to 4.0 wt %, even more preferably from 0.3 wt % to 2.0 wt % of at least one solubilizer. Solubilizers are listed above. In certain embodiments, the solubilizer is selected from the group consisting of PEG-40 hydrogenated castor oil, polysorbate 20, poloxamer 407, and combinations thereof.

Optionally the mouthwash composition comprises from 0.001 wt % to 5.0 wt %, preferably from 0.05 wt % to 3.0 wt %, even more preferably from 0.1 wt % to 1.0 wt % of at least one electrolyte. Electrolytes are listed above. In certain embodiments, the electrolyte is selected from the group consisting of sodium fluoride, sodium monofluorophosphate, stannous fluoride, and combinations thereof.

Optionally the mouthwash composition comprises from 0.01 wt % to 5.0 wt %, preferably from 0.1 wt % to 3.0 wt %, even more preferably from 0.4 wt % to 1.0 wt % of at least one preservative or preservation boosting ingredient. Preservatives or preservation boosting ingredients are listed above. In certain embodiments, the preservative or preservation boosting ingredient is selected from the group consisting of sodium benzoate, benzoic acid, chlorhexidine, and combinations thereof.

Optionally the mouthwash composition comprises from 0.01 wt % to 20 wt %, preferably from 0.1 wt % to 10 wt %, even more preferably from 0.5 wt % to 5.0 wt % of at least one further auxiliary. Auxiliaries are listed above. In a preferred embodiment, the mouthwash composition has a pH value from 2.0 to 10.0, preferably from 3.0 to 9.0, even more preferably from 4.0 to 8.0.

### Fourth Aspect

A fourth aspect relates to a method of caring for keratinous material, comprising:
(1) Applying the cosmetic composition as otherwise described herein (e.g., according to the third aspect) or the antimicrobial composition as otherwise described herein (e.g., according to the first aspect onto keratinous material); and
(2) Allowing the composition to remain on the keratinous material for at least 1 hour.

In certain embodiments, the method comprises applying the cosmetic composition as otherwise described herein onto keratinous material); and allowing the composition to remain on the keratinous material for at least 1 hour.

In certain embodiments, the method as otherwise described herein does not comprise a rinsing step (e.g., by refraining from rinsing the treated skin for at least 1 hour, at least 2 hours, at least 4 hours, or at least 6 hours).

### Fifth Aspect

A fifth aspect relates to the use of the antimicrobial combination composition of the first aspect, in preserving cosmetic compositions.

### EXAMPLES

The examples which follow are intended to illustrate the subject matter of the invention without restricting it thereto.

Several antimicrobial combinations compositions were formulated to pH of 5.5. These compositions were tested for their ability to inhibit in vitro the growth of a microbial strain. The results of these studies are provided in Table 1. “Green” indicates a substantial inhibition, “Yellow” indicates an intermediate inhibition, and “Red” indicates little to no inhibition.

TABLE 1
Example compositions and their antimicrobial activity

| | Pseudomonas | Staphylococcus | Echerichia | Candida | Aspergillus |
|---|---|---|---|---|---|
| | aeruginosa | aureus | coli | albicans | brasilliensis |
| Blank | Green | Yellow | Red | Red | Red |
| 0.5% Velsan | Green | Green | Green | Green | Red |
| ethylhexylglycerin | | | | | |
| 0.8% Velsan | Green | Green | Green | Green | Red |
| ethylhexylglycerin | | | | | |
| 1.0% Velsan | Green | Green | Green | Green | Red |
| ethylhexylglycerin | | | | | |
| 1.5% Velsan | Green | Green | Green | Green | Red |
| ethylhexylglycerin | | | | | |
| 0.5% Octylglycerin | Green | Green | Green | Green | Yellow |
| 0.8% Octylglycerin | Green | Green | Green | Green | Green |
| 1.0% Octylglycerin | Green | Green | Green | Green | Yellow |
| 1.5% Octylglycerin | Green | Green | Green | Green | Yellow |
| 0.5% Octylglycerin + | Green | Green | Green | Green | Green |
| naringenin | | | | | |
| 0.8% Octylglycerin + | Green | Green | Green | Green | Green |
| naringenin | | | | | |
| 1.0% Octylglycerin + | Green | Green | Green | Green | Green |
| naringenin | | | | | |
| 1.5% Octylglycerin + | Green | Green | Green | Green | Green |
| naringenin | | | | | |

Various exemplary embodiments of the disclosure include, but are not limited to:

Embodiment 1 provides an antimicrobial combination composition comprising:
(a) one or more glycerol derivatives selected from the group consisting of: 3-[(2-ethylhexyl)oxy]-1,2-propanediol, 3-[(n-octyl)oxy]-1,2-propanediol, and 3-[(2-octyl)oxy]-1,2-propanediol; and
(b) one or more bicyclic compounds selected from the group consisting of: 5,7-dihydroxy-2-(4-hydroxyphenyl)chroman-4-one (naringenin), 2-(3,4-dihydroxyphenyl)-3,5,7-trihydroxychromen-4-one (quercetin), 5,7-dihydroxy-2-(3-hydroxy-4-methoxyphenyl)chroman-4-one (hesperetin), and 5,7-dihydroxy-2-(3,4-dihydroxyphenyl)-chroman-4-one (eriodyctiol).

Embodiment 2 provides the antimicrobial composition of embodiment 2, wherein the glycerol derivative is selected from the group consisting of: 3-[(n-octyl)oxy]-1,2-propanediol, and 3-[(2-octyl)oxy]-1,2-propanediol; or the one or more glycerol derivatives is 3-[(n-octyl)oxy]-1,2-propanediol and 3-[(2-octyl)oxy]-1,2-propanediol; or the one or more glycerol derivatives is 3-[(n-octyl)oxy]-1,2-propanediol; or the one or more glycerol derivatives is 3-[(2-octyl)oxy]-1,2-propanediol; or the one or more glycerol derivatives is 3-[(2-ethylhexyl)oxy]-1,2-propanediol; or the glycerol derivative may be selected from the compounds of formula (I) provided in International Patent Publication WO 2018/125734.

Embodiment 3 provides the antimicrobial composition according to embodiment 1 and 2, wherein the antimicrobial combination composition is a concentrate or a working mixture.

Embodiment 4 provides the antimicrobial composition according to any one of embodiments 1-3, wherein the glycerol derivative of the disclosure is provided at a purity of at least 99.99%, or at least 99.90%, or at least 99%, or at least 98%, or at least 94%, prior to its introduction into the antimicrobial combination composition.

Embodiment 5 provides the antimicrobial composition according to any one of embodiments 1-4, wherein the bicyclic compound is selected from the group consisting of: naringenin, hesperetin, and hesperetin; or the one of more bicyclic compounds is naringenin; or the one of more bicyclic compounds is quercetin; or the one of more bicyclic compounds is hesperetin; or the one of more bicyclic compounds is eriodyctiol; or the one of more bicyclic compounds is naringenin and quercetin; or the one of more bicyclic compounds is naringenin and hesperetin; or the one of more bicyclic compounds is naringenin and eriodyctiol; or the bicyclic compound may be selected from the compounds of formula (II) provided in International Patent Publication WO 2018/125734.

Embodiment 6 provides the composition according to any one of embodiments 1-5, wherein the bicyclic compound of the disclosure is provided at a purity of at least 99.99%, or at least 99.90%, or at least 99%, or at least 98%, or at least 94%, or at least 92%, or at least 90%, or at least 89%, or at least 87%, or at least 85% prior to its introduction into the antimicrobial combination composition.

Embodiment 7 provides the antimicrobial composition according to any one of embodiments 1-6, wherein the one or more bicyclic compounds is present in an amount in the range of about 50 ppm to about 50000 ppm (equivalent to 0.005 wt % to 5 wt %), based on the total weight of the one or more glycerol derivatives; or in an amount in the range of about 100 ppm to about 10000 ppm, or about 200 ppm to about 5000 ppm, or about 500 ppm to about 1000 ppm, or about 300 ppm to about 900 ppm, based on the total weight of the one or more glycerol derivatives.

Embodiment 8 provides the antimicrobial composition according to any one of embodiments 1-7, wherein the one or more bicyclic compounds is present in an amount less than 5 wt. %, or less than 3 wt. %, or less than 1 wt. %, or even less than 0.5 wt. %, based on the total weight of the one or more glycerol derivatives; or the one or more bicyclic compounds is naringenin (5,7-dihydroxy-2-(4-hydroxyphenyl)chroman-4-one) present in an amount less than 5 wt. %, or less than 3 wt. %, or less than 1 wt. %, or even less than 0.5 wt. %, based on the total weight of the one or more glycerol derivatives.

Embodiment 9 provides the antimicrobial composition according to any one of embodiments 1-8, wherein (a) one or more glycerol derivatives selected from the group consisting of: 3-[(n-octyl)oxy]-1,2-propanediol, and 3-[(2-octyl)oxy]-1,2-propanediol; and (b) one or more bicyclic compounds is naringenin.

Embodiment 10 provides the antimicrobial composition according to any one of embodiments 1-8, wherein (a) one or more glycerol derivatives selected from the group consisting of 3-[(2-ethylhexyl)oxy]-1,2-propanediol; and (b) one or more bicyclic compounds is naringenin.

Embodiment 11 provides the antimicrobial composition according to any one of embodiments 1-8, wherein (a) one or more glycerol derivatives is 3-[(n-octyl)oxy]-1,2-propanediol; and (b) one or more bicyclic compounds is naringenin.

Embodiment 12 provides the antimicrobial composition according to any one of embodiments 1-8, wherein (a) one or more glycerol derivatives is 3-[(2-octyl)oxy]-1,2-propanediol; and (b) one or more bicyclic compounds is naringenin.

Embodiment 13 provides the antimicrobial composition according to any one of embodiments 1-8, wherein (a) one or more glycerol derivatives selected from the group consisting of: 3-[(n-octyl)oxy]-1,2-propanediol, and 3-[(2-octyl)oxy]-1,2-propanediol; and (b) one or more bicyclic compounds is quercetin.

Embodiment 14 provides the antimicrobial composition according to any one of embodiments 1-8, wherein (a) one or more glycerol derivatives is 3-[(2-ethylhexyl)oxy]-1,2-propanediol; and (b) one or more bicyclic compounds is quercetin.

Embodiment 15 provides the antimicrobial composition according to any one of embodiments 1-8, wherein (a) one or more glycerol derivatives is 3-[(n-octyl)oxy]-1,2-propanediol; and (b) one or more bicyclic compounds is quercetin.

Embodiment 16 provides the antimicrobial composition according to any one of embodiments 1-8, wherein (a) one or more glycerol derivatives is 3-[(2-octyl)oxy]-1,2-propanediol; and (b) one or more bicyclic compounds is quercetin.

Embodiment 17 provides the antimicrobial composition according to any one of embodiments 1-8, wherein (a) one or more glycerol derivatives selected from the group consisting of: 3-[(n-octyl)oxy]-1,2-propanediol, and 3-[(2-octyl)oxy]-1,2-propanediol; and (b) one or more bicyclic compounds is hesperetin.

Embodiment 18 provides the antimicrobial composition according to any one of embodiments 1-8, wherein (a) one or more glycerol derivatives is 3-[(2-ethylhexyl)oxy]-1,2-propanediol; and (b) one or more bicyclic compounds is hesperetin.

Embodiment 19 provides the antimicrobial composition according to any one of embodiments 1-8, wherein (a) one or more glycerol derivatives is 3-[(n-octyl)oxy]-1,2-propanediol; and (b) one or more bicyclic compounds is hesperetin.

Embodiment 20 provides the antimicrobial composition according to any one of embodiments 1-8, wherein (a) one or more glycerol derivatives is 3-[(2-octyl)oxy]-1,2-propanediol; and (b) one or more bicyclic compounds is hesperetin.

Embodiment 21 provides the antimicrobial composition according to any one of embodiments 1-8, wherein (a) one or more glycerol derivatives selected from the group consisting of: 3-[(n-octyl)oxy]-1,2-propanediol, and 3-[(2-octyl)oxy]-1,2-propanediol; and (b) one or more bicyclic compounds is eriodyctiol.

Embodiment 22 provides the antimicrobial composition according to any one of embodiments 1-8, wherein (a) one or more glycerol derivatives is 3-[(2-ethylhexyl)oxy]-1,2-propanediol; and (b) one or more bicyclic compounds is eriodyctiol.

Embodiment 23 provides the antimicrobial composition according to any one of embodiments 1-8, wherein (a) one or more glycerol derivatives is 3-[(n-octyl)oxy]-1,2-propanediol; and (b) one or more bicyclic compounds is eriodyctiol.

Embodiment 24 provides the antimicrobial composition according to any one of embodiments 1-8, wherein (a) one or more glycerol derivatives is 3-[(2-octyl)oxy]-1,2-propanediol; and (b) one or more bicyclic compounds is eriodyctiol.

Embodiment 25 provides the antimicrobial composition according to any one of embodiments 1-24, in the form of a concentrate (e.g., that can be further used in a cosmetic formulation or a pharmaceutical formulation).

Embodiment 26 provides the antimicrobial composition according to any one of embodiments 1-24, in the form of a working solution.

Embodiment 27 provides the antimicrobial composition according to embodiment 26, wherein the antimicrobial combination composition is further diluted to concentration of 10 wt % to 60 wt % in order to provide working solution.

Embodiment 28 provides the antimicrobial composition according to embodiment 26 or 27, wherein the antimicrobial combination composition is further diluted with an additive (e.g., a diluent or an excipient).

Embodiment 29 provides the antimicrobial composition according to embodiment 28, wherein the additive is water, alcohol(s), polyol(s), or a mixture thereof.

Embodiment 30 provides the antimicrobial composition according to any one of embodiments 1-29, which is substantially free or completely free of one or more peroxides (e.g., less than 1000 ppm, or less than 100 ppm, or less than 10 ppm, or even less than 1 ppm of peroxide(s)).

Embodiment 31 provides the antimicrobial composition according to any one of embodiments 1-30, wherein the composition is part of a deodorant, skincare product, sunscreen, baby product, cosmetic, aftershave, disinfectant, antiseptic, washing lotion, in-shower lotion, and hair treatment composition (e.g., leave-in or rinse out composition).

Embodiment 32 provides the antimicrobial composition according to any one of embodiments 1-31 for use as a medicament.

Embodiment 33 provides the antimicrobial composition according to any one of embodiments 1-31 for use in (prophylactically) treating infections on the human or animal body.

Embodiment 34 provides a cosmetic composition comprising the antimicrobial combination composition according to any of embodiments 1-30; specifically, embodiment 34 provides a cosmetic composition comprising:
(a) one or more glycerol derivatives selected from the group consisting of: 3-[(2-ethylhexyl)oxy]-1,2-propanediol, 3-[(n-octyl)oxy]-1,2-propanediol, and 3-[(2-octyl)oxy]-1,2-propanediol; and
(b) one or more bicyclic compounds selected from the group consisting of: 5,7-dihydroxy-2-(4-hydroxyphenyl)chroman-4-one (naringenin), 2-(3,4-dihydroxyphenyl)-3,5,7-trihydroxychromen-4-one (quercetin), 5,7-dihydroxy-2-(3-hydroxy-4-methoxyphenyl)chroman-4-one (hesperetin), and 5,7-dihydroxy-2-(3,4-dihydroxyphenyl)-chroman-4-one (eriodyctiol).

Embodiment 35 provides the cosmetic composition of embodiment 34 having pH value in the range of 2.0 to 7.0, or 3.0 to 7.0, or 4.5 to 7.0, or pH 4 to pH 6.5, or pH 4 to pH 6, or pH 4.5 to pH 6.5, or pH 4.5 to pH 6, or pH 5.3 to pH 5.7, or pH 5 to pH 6, or pH of about 5.5.

Embodiment 36 provides the cosmetic composition of embodiment 34 or 35 for leave-on skin care use.

Embodiment 37 provides the cosmetic composition of any of embodiments 34-36, wherein the antimicrobial combination composition is present in an amount of up to about 60% by weight, based on the total weight of the cosmetic composition; or in the range of 1 wt % to 60 wt %, or 10 wt % to 60 wt %, or 20 wt % to 60 wt %, or 30 wt % to 60 wt %, or 40 wt % to 60 wt %,or 50 wt % to 60 wt %, or 1 wt % to 40 wt %, or 10 wt % to 40 wt %, or 20 wt % to 40 wt %, or 30 wt % to 40 wt %, or 1 wt % to 30 wt %, or 10 wt % to 30 wt %, or 20 wt % to 30 wt %, or 1 wt % to 20 wt %, or 10 wt % to 20 wt %, or 15 wt % to 20 wt %, or 1 wt % to 15 wt %, or 1 wt % to 10 wt %, or 1 wt % to 5 wt % of the antimicrobial combination composition, based on the total weight of the cosmetic composition.

Embodiment 37-a provides the cosmetic composition of any of embodiments 34-36, wherein the amount of the one or more glycerol derivatives present in the cosmetic composition is in the range of about 0.0005 wt % to about 5 wt %; e.g., in the range of about 0.001 wt % to about 5 wt %, or from about 0.01 wt % to about 5 wt %, or from about 0.1 wt % to about 5 wt %, or from about 1 wt % to about 5 wt %, or 0.0005 wt % to about 1 wt %, or from about 0.001 wt % to about 1 wt %, or from about 0.01 wt % to about 1 wt %, or from about 0.1 wt % to about 1 wt %, or from about 0.5 wt % to about 1 wt %, or 0.0005 wt % to about 0.5 wt %, or from about 0.001 wt % to about 0.5 wt %, or from about 0.01 wt % to about 0.5 wt %, or from about 0.1 wt % to about 0.5 wt %, or from about 0.5 wt % to about 0.5 wt %, or 0.0005 wt % to about 0.1 wt %, or from about 0.001 wt % to about 0.1 wt %, or from about 0.01 wt % to about 0.1 wt %, about 0.05 wt % to about 5 wt %, or from about 0.1 wt % to about 1 wt %, or from about 0.1 wt % to about 0.6 wt %, or from about 0.1 wt % to about 0.5 wt %, or from about 0.1 wt % to about 0.4 wt %, or from about 0.2 wt % to about 1 wt %, or from about 0.2 wt % to about 0.6 wt %, or from about 0.2 wt % to about 0.5 wt %, or from about 0.2 wt % to about 0.4 wt %, or from about 0.4 wt % to about 1 wt %, or from about 0.4 wt % to about 0.6 wt %, or from about 0.4 wt % to about 0.5 wt %, or about 0.3 wt %, or about 0.5%, based on the total weight of the cosmetic composition.

Embodiment 37-b provides the cosmetic composition of any of embodiments 34-37a, wherein the amount of the one or more bicyclic compounds present in the cosmetic composition is in the range of about 50 ppm to about 50000 ppm (equivalent to 0.005 wt % to 5 wt %), based on the total weight of the one or more glycerol derivatives present in the cosmetic composition; e.g., in the range of about 100 ppm to about 10000 ppm, or about 200 ppm to about 5000 ppm, or about 500 ppm to about 1000 ppm, or about 300 ppm to about 900 ppm, based on the total weight of the one or more glycerol derivatives present in the cosmetic composition.

Embodiment 37-c provides the cosmetic composition of any of embodiments 34-37b, wherein the amount of the one or more bicyclic compounds present in the cosmetic composition includes an additional amount of the one or more bicyclic compounds (i.e., in addition to those provided with respect to the antimicrobial combination composition).

Embodiment 38 provides the cosmetic composition of any of embodiments 34-37c, further comprising one or more additives selected from surfactants and auxiliary agents.

Embodiment 39 provides the cosmetic composition of embodiment 38, wherein the cosmetic composition comprises one or more surfactants.

Embodiment 40 provides the cosmetic composition of embodiment 38 or 39, wherein the one or more surfactants is selected from the group consisting of anionic surfactants, cationic surfactants, non-ionic surfactants, zwitterionic surfactants and/or amphoteric surfactants, all as provided herein.

Embodiment 41 provides the cosmetic composition of any of embodiments 38-40, wherein the one or more surfactants in present in an amount of from 0.01 wt % to 70 wt %, from 0.1 wt % to 40%, from 1 wt % to 30%, from 2 wt % to 20 wt %, based on the total weight of the cosmetic composition.

Embodiment 42 provides the cosmetic composition of any of embodiments 38-41 further comprising a fatty acyl isethionate as otherwise described herein (e.g., a level of from 1 to 10 wt %, or from 2 to 8 wt %, or from 2.5 to 7.5 wt % based on the total weight of the composition)

Embodiment 43 provides the cosmetic composition of any of embodiments 38-42, wherein the one or more surfactant includes one or more of acylglycinate surfactant (e.g., such as the compound of formula (Y)), glutamate surfactant (e.g., such as the compound of formula (Z)), for example in each in an amount of from 0.01 wt % to 30 wt %, or 1 wt % to 25 wt %, or from 5 wt % to 20 wt %, or from 12 wt % to 18 wt % based on the total weight of the composition.

Embodiment 44 provides the cosmetic composition of any of embodiments 38-43, wherein the one or more surfactaint includes one or more nonionic surfactants, such as alkyl ethoxylates, alkyl polyglycosides (APGs), ethoxylated fatty alcohols, fatty acids, fatty acid glycerides or alkylphenols, fatty alcohol ethoxylates (alkylpolyethylene glycols), alkylphenol polyethylene glycols, alkylmercaptan polyethylene glycols, fatty amine ethoxylates (alkylaminopolyethylene glycols), fatty acid ethoxylates (acylpolyethylene glycols), polypropylene glycol ethoxylates (Pluronicsl, fatty acid alkylol amides, (fatty acid amide polyethylene glycols), N-alkyl-, N-alkoxypolyhydroxy-fatty acid amide, sucrose esters, sorbitol esters, polyglycol ethers, ans sugar-derived non-ionic surfactants as described herein (e.g., such as the compound of formula (X)).

Embodiment 45 provides the cosmetic composition of embodiments 44, wherein the cosmetic composition comprises from 1 wt % to 20 wt %, more preferably from 2 wt % to 10 wt %, even more preferably from 3 wt % to 7 wt % non-ionic surfactant.

Embodiment 46 provides the cosmetic composition of any of embodiments 38-45, wherein the one or more surfactants includes an amphoteric or zwitterionic surfactant(s), e.g., in an amount ranging from 0.5 wt % to about 8 wt %, preferably from 1 wt % to 4 wt % of the total composition.

Embodiment 47 provides the cosmetic composition of embodiments 46, wherein the amphoteric surfactants are selected from the group consisting of N—(C₁₂-C₁₈)-alkyl-β-aminopropionates and N—(C₁₂-C₁₈)-alkyl-β-iminodipropionates as alkali metal salts and mono-, di-, and trialkylammonium salts; N-acylaminoalkyl-N,N-dimethylacetobetaine, preferably N—(C₈-C₁₈)-acylaminopropyl-N,N-dimethylacetobetaine, (C₁₂-C₁₈)-alkyl-dimethylsulfopropylbetaine, amphosurfactants based on imidazoline (trade name: Miranol®, Steinapon®), preferably the sodium salt of 1-((3-carboxymethyloxyethyl)-1-(carboxymethyl)-2-laurylimidazolinium; amine oxide, e.g., (C₁₂-C₁₈)-alkyl-dimethylamine oxide, fatty acid amidoalkyldimethylamine oxide, and mixtures thereof.

Embodiment 48 provides the cosmetic composition of any of embodiments 38-47, wherein the one or more surfactants includes a betaine surfactant, such as cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and laurylbis(2-hydroxypropyl)alphacarboxyethylbetaine, cocodimethylsulfopropylbetaine, stearyldimethylsulfopropylbetaine, lauryldimethyl-sulfoethylbetaine, laurylbis(2-hydroxyethyl)sulfopropylbetaine, carboxyl derivatives of imidazole, the C₈- to C₁₈-alkyldimethylammonium acetates, the C₈- to C₁₈-alkyldimethylcarbonylmethylammonium salts, coconut fatty acid amidopropylbetaine, N-coconut fatty acid amidoethyl-N-[2-(carboxymethoxy)ethyl]glycerol (CTFA name: Cocoamphocarboxyglycinate), and mixtures thereof.

Embodiment 49 provides the cosmetic composition of any of embodiments 38-48, wherein the composition further comprises a surfactant system (e.g., the surfactant system comprises at least one surfactant selected from the group consisting of lauryl sulfate, laureth sulfate, cocoamidopropylbetaine, sodium cocoylglutamate, lauroamphoacetate, and mixtures thereof; or comprises sodium laureth sulphate, sodium lauryl sulphate, and optionally cocamidopropyl betaine; or comprises sodium laureth sulphate, Potassium Cocyl Glutamate, and cocamidopropyl betaine.)

Embodiment 50 provides the cosmetic composition of any of embodiments 38-49, wherein the composition comprises one or more auxiliary agents, e.g., cosmetically acceptable auxiliary agents.

Embodiment 51 provides the cosmetic composition of embodiment 50, wherein the one or more auxiliary agents is selected from the group consisting of oily substances, waxes, emulsifiers, coemulsifiers, solubilizers, cationic polymers, film formers, superfatting agents, refatting agents, foam stabilizers, stabilizers, active biogenic substances, preservatives, preservation boosting ingredients, anti-fungal substances, anti-dandruff agents, dyes or pigments, particulate substances, opacifiers, abrasives, absorbents, anticaking agents, bulking agents, pearlizing agents, direct dyes, perfumes or fragrances, carriers, solvents or diluents, propellants, functional acids, active ingredients, skin-brightening agents, self-tanning agents, exfoliants, enzymes, anti-acne agents, deodorants and anti-perspirants, viscosity modifiers, thickening and gelling agents, pH adjusting agents, buffering agents, anti-oxidants, chelants, astringents, sunscreens, sun protection agents, UV filters, skin conditioning agents, emollients, humectants, occlusive agents, pediculocides, anti-foaming agents, flavouring agents, electrolytes, oxidizing agents and reducing agents, all as described herein.

Embodiment 52 provides the cosmetic composition of embodiment 50 or 51, wherein the one or more auxiliary agents includes an oily substance as described herein, e.g., from 0.001 wt % to 60 wt %, preferably from 0.05 wt % to 50 wt %, even more preferably from 0.1 wt % to 40 wt % based on the total weight of the composition

Embodiment 53 provides the cosmetic composition of any one of embodiments 50-52, wherein the one or more auxiliary agents includes a wax as described herein, e.g., from 0.001 wt % to 30 wt %, preferably from 0.05 wt % to 20 wt %, even more preferably from 0.1 wt % to 10 wt % based on the total weight of the composition.

Embodiment 54 provides the cosmetic composition of any one of embodiments 50-53, wherein the one or more auxiliary agents is selected from an emulsifier, coemulsifier, and solubilizer as otherwise described herein, e.g., from 0.1 wt % to 20 wt %, preferably from 0.5 wt % to 10 wt %, even more preferably from 1.0 wt % to 5.0 wt % based on the total weight of the composition.

Embodiment 55 provides the cosmetic composition of embodiment 54, wherein the emulsifier, coemulsifier, and solubilizer is selected from the group consisting of glyceryl stearate, cetearyl alcohol, polysorbate 20, stearic acid, cetearyl glucoside, PEG-40 hydrogenated castor oil, cetyl phosphate, steareth-2, ceteth-10 phosphate, trilaureth-4 phosphate, polyglyceryl-2 sesquiisostearate, cetyl PEG/PPG-10/1 dimethicone, cetrimonium chloride, and combinations thereof.

Embodiment 56 provides the cosmetic composition of any one of embodiments 50-55, wherein the one or more auxiliary agents is selected from a cationic polymer as described herein, a film former as otherwise described herein, a superfatting agent and/or a refatting agent as otherwise described herein, e.g., from 0.1 wt % to 10 wt %, preferably from 0.5 wt % to 7.5 wt %, preferably from 1.0 wt % to 5.0 wt %, even more preferably from 0.5 wt % to 3.0 wt %, based on the total weight of the composition.

Embodiment 57 provides the cosmetic composition of any one of embodiments 50-56, wherein the one or more auxiliary agents includes a stabilizer as otherwise described herein, e.g., from 0.01 wt % to 10 wt %, preferably from 0.5 wt % to 8.0 wt %, even more preferably from 1.0 wt % to 5.0 wt %, based on the total weight of the composition.

Embodiment 58 provides the cosmetic composition of any one of embodiments 50-57, wherein the one or more auxiliary agents includes a biogenic substance as otherwise described herein, e.g., from 0.001 wt % to 5.0 wt %, preferably from 0.01 wt % to 3.0 wt %, even more preferably from 0.1 wt % to 2.0 wt %, based on the total weight of the composition.

Embodiment 59 provides the cosmetic composition of any one of embodiments 50-58, wherein the one or more auxiliary agents is selected from a preservative, preservation boosting ingredient, anti-fungal agent, and anti-dandruff agent, all as described herein. For example, the composition may include 0.01 to 5.0 wt %, particularly preferably from 0.05 wt % to 1.0 wt % of preservative; and/or from 0.1 wt % to 1.0 wt % of anti-fungal agent; and/or from 0.1% to 4.0%, preferably from 0.1% to 3.0%, more preferably from 0.3% to 2.0% anti-dandruff agent.

Embodiment 60 provides the cosmetic composition of any one of embodiments 38-59, wherein the composition further includes from 0.1 wt % to 5.0 wt % of additional antimicrobial agents (such as chlorhexidine)

Embodiment 61 provides the cosmetic composition of any one of embodiments 50-60, wherein the one or more auxiliary agents is selected from a dye or pigment, a particulate substance, a pearlizing agent, a direct dye, and a perfume or fragrance ingredient. For example, the composition may include from 0.01 wt % to 20 wt %, preferably from 0.05 wt % to 15 wt %, even more preferably from 0.5 wt % to 10 wt % of a particulate substance; and/or from 0.1 wt % to 15 wt %, preferably from 0.5 wt % to 10 wt %, even more preferably from 1.0 wt % to 5.0 wt % of a pearlizing agent; and/or from 0.1 wt % to 15 wt %, preferably from 0.1 wt % to 10 wt %, even more preferably from 0.5 wt % to 8.0 wt % of a direct dye; and/or from 0.01 wt % to 3.0 wt %, preferably from 0.05 wt % to 2.0 wt %, even more preferably from 0.1 wt % to 1.0 wt % of a perfume or fragrance.

Embodiment 62 provides the cosmetic composition of any one of embodiments 50-60, wherein the one or more auxiliary agents includes a carrier, solvent, and/or diluent.

Embodiment 63 provides the cosmetic composition of embodiment 62, wherein the solvent comprises water and/or alcohol as otherwise described hereing (e.g., water-miscible or water-soluble alcohol such as C₁-C₅ alkyl monohydric alcohols or polyhydric alcohols); or the solvent is water.

Embodiment 64 provides the cosmetic composition of embodiment 62 or 63, wherein the solvent comprises plant oil, honey, plant-derived sugar compositions, and mixtures thereof.

Embodiment 65 provides the cosmetic composition of any one of embodiments 62-64, wherien the cosmetic composition comprises from 0.5 wt % to 90 wt %, preferably from 1.0 wt % to 80 wt %, even more preferably from 5.0 wt % to 70 wt % of at least one carrier, solvent, and/or diluent.

Embodiment 66 provides the cosmetic composition of any one of embodiments 50-65, wherein the one or more auxiliary agents includes a propellant as described herein, e.g., from 0.5 wt % to 60 wt %, preferably from 1.0 wt % to 50 wt %, even more preferably from 2.0 wt % to 40 wt %, based on the total weight of the composition.

Embodiment 67 provides the cosmetic composition of any one of embodiments 50-66, wherein the one or more auxiliary agents includes a functional acid as described herein and/or an active ingredient as described herein, e.g., from 0.05 wt % to 15 wt %, preferably from 0.1 wt % to 10 wt %, even more preferably from 0.5 wt % to 5.0 wt %, based on the total weight of the composition.

Embodiment 68 provides the cosmetic composition of any one of embodiments 50-67, wherein the one or more auxiliary agents includes a deodorant as described herein and/or an anti-perspirants as described herein. For example, the cosmetic composition comprises from 0.001 wt % to 10 wt %, or from 0.01 wt % to 9.0 wt %, or from 0.05 wt % to 8.0 wt %, or from 0.1 wt % to 5.0 wt % of a deodorant; and from 0.001 wt % to 10 wt %, or from 0.01 wt % to 9.0 wt %, or from 0.05 wt % to 8.0 wt %, or from 0.1 wt % to 5.0 wt % of an antiperspirant.

Embodiment 69 provides the cosmetic composition of any one of embodiments 50-68, wherein the one or more auxiliary agents includes a viscosity modifier and/or thickening agent and/or gelling agent, all as described herein, e.g., from 0.01 wt % to 15 wt %, preferably from 0.1 wt % to 10 wt %, even more preferably from 0.5 wt % to 5.0 wt %, based on the total weight of the composition.

Embodiment 70 provides the cosmetic composition of any one of embodiments 50-69, wherein the one or more auxiliary agents includes an alkalizing agent as described herein and/or pH adjusting agent as described herein; e.g., from 0.001 wt % to 5.0 wt %, preferably from 0.01 wt % to 3.0 wt %, even more preferably from 0.1 wt % to 1.0 wt %, based on the total weight of the composition.

Embodiment 71 provides the cosmetic composition of any one of embodiments 50-70, wherein the one or more auxiliary agents includes an anti-oxidant as otherwise described herein; e.g., from 0.001 wt % to 10 wt %, preferably from 0.05 wt % to 5.0 wt %, even more preferably from 0.1 wt % to 3.0 wt %, most preferably from 0.05 wt % to 1.0 wt %, based on the total weight of the composition.

Embodiment 72 provides the cosmetic composition of any one of embodiments 50-71, wherein the one or more auxiliary agents includes a chelant as otherwise described herein; e.g., from 0.01 wt % to 2.0 wt %, preferably from 0.05 wt % to 1.5 wt %, even more preferably from 0.1 wt % to 1.0 wt %, most preferably from 0.05 wt % to 1.0 wt %, based on the total weight of the composition.

Embodiment 73 provides the cosmetic composition of any one of embodiments 50-72, wherein the one or more auxiliary agents includes an astringent as otherwise described herein; e.g., from 0.001 wt % to 10 wt %, or from 0.01 wt % to 9.0 wt %, or from 0.05 wt % to 8.0 wt %, or from 0.1 wt % to 5.0 wt %, based on the total weight of the composition.

Embodiment 74 provides the cosmetic composition of any one of embodiments 50-73, wherein the one or more auxiliary agents includes a sun protection agent and/or UV filter as otherwise described herein; e.g., 0.001 wt % to 30 wt %, preferably from 0.05 wt % to 20 wt %, even more preferably from 0.1 wt % to 10 wt %, most preferably from 0.05 wt % to 5.0 wt %, based on the total weight of the composition.

Embodiment 75 provides the cosmetic composition of any one of embodiments 50-74, wherein the one or more auxiliary agents includes a skin conditioning agent as otherwise described herein; e.g., from 0.001 wt % to 5.0 wt %, preferably from 0.05 wt % to 3.0 wt %, even more preferably from 0.1 wt % to 1.0 wt %, based on the total weight of the composition.

Embodiment 76 provides the cosmetic composition of any one of embodiments 50-75, wherein the one or more auxiliary agents includes an anti-foaming agent as otherwise described herein; e.g., from 0.01 wt % to 5.0 wt %, preferably from 0.1 wt % to 3.0 wt %, even more preferably from 0.5 wt % to 2.0 wt %, based on the total weight of the composition.

Embodiment 77 provides the cosmetic composition of any one of embodiments 50-76, wherein the one or more auxiliary agents includes a flavouring agent as otherwise described herein and/or an electrolyte as otherwise described herein; e.g., from 0.001 wt % to 5.0 wt %, or from 0.001 wt % to 3.0 wt %, or from 0.05 wt % to 3.0 wt %, or from 0.01 wt % to 2.0 wt %, or from 0.1 wt % to 1.0 wt %, or from 0.05 wt % to 1.0 wt %, based on the total weight of the composition.

Embodiment 78 provides the cosmetic composition of any one of embodiments 50-77, wherein the one or more auxiliary agents includes an oxidizing agent as otherwise described herein or a reducing agent as otherwise described herein; e.g., from 0.001 wt % to 10 wt %, preferably from 0.05 wt % to 7.0 wt %, even more preferably from 0.1 wt % to 5.0 wt %, based on the total weight of the composition.

Embodiment 79 provides the cosmetic composition of any one of embodiments 34-78, wherein the composition has a viscosity of from 0 cPs to 20,000 cPs; or from 0.1 cPs to 10,000 cPs, or from 1 cPs to 5,000 cPs, or from 5 cPs to 3,500 cPs; or from 0 cPs to 1,000 cPs.

Embodiment 80 provides the cosmetic composition of any one of embodiments 34-79, wherein the composition further comprises a viscosity-modifying substance (such as a thickening polymer.)

Embodiment 81 provides the cosmetic composition of any one of embodiments 34-79, wherein the composition is in liquid form.

Embodiment 82 provides the cosmetic composition of any one of embodiments 34-79, wherein the composition is in solid form; e.g., in powdered or granulated form.

Embodiment 83 provides the cosmetic composition of any one of embodiments 34-82, wherein the composition is selected from the group consisting of shampoo, conditioner (e.g., leave-in or rinse out), body wash, facial cleanser, face mask, bubble bath, intimate wash, bath oil, cleansing milk, micellar water, make-up remover, cleansing wipes, hair mask, perfume, liquid soap, shaving soap, shaving foam, cleansing foam, day cream, anti-ageing cream, body milk, body lotion, in-shower body lotion (i.e., no rinse body lotion), body mousse, face serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm (bb) cream, eyeliner, night cream, eye brow gel, highlighter, lip stain, hand sanitizer, hair oil, nail varnish remover, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, body scrub, cellulite treatment, bar soap, cuticle cream, lip balm, hair treatment, eye shadow, bath additive, body mist, eau de toilette, mouthwash, toothpaste, lubricating gel, moisturizer, serum, toner, aqua sorbet, cream gel, styling mousse, dry shampoo, lip stick, lip gloss, hydro-alcoholic gel, body oil, shower milk, illuminator, lip crayon, hair spray, combing cream, and sunblock.

Embodiment 84 provides the cosmetic composition of any one of embodiments 34-83, wherein the composition is for use on skin, such as for use on the face, the neck, the body and/or around the eye area; e.g., the composition is an emulsion or gel, preferably an oil-in-water (o/w), cream gel, hydro-alcoholic gel or hydrogel composition.

Embodiment 85 provides the cosmetic composition of any one of embodiments 34-83, wherein the composition is a body or face care composition such as face creams, neck creams, body lotions, body milks, face serums, blemish balm creams, hand creams, foot creams, body butters, lip creams, eye creams, after-sun lotions, make-up removing lotions or body mists, diaper creams or baby lotions; and for example, optionally comprises from 0.1 wt % to 15 wt %, preferably from 0.5 wt % to 10 wt %, even more preferably from 1.0 wt % to 5.0 wt % of at least one emulsifier, coemulsifier and/or solubilizer; and/or optionally comprises from 0.01 wt % to 40 wt %, preferably from 0.05 wt % to 30 wt %, even more preferably from 0.1 wt % to 20 wt % of at least one oily substance; and/or optionally comprises from 0.01 wt % to 20 wt %, preferably from 0.05 wt % to 10 wt %, even more preferably from 0.1 wt % to 5.0 wt % of at least one wax; and/or optionally comprises from 0.01 wt % to 15 wt %, preferably from 0.1 wt % to 10 wt %, even more preferably from 0.3 wt % to 5.0 wt % of at least one viscosity modifier or thickening and/or gelling agent; and/or optionally comprises from 0.001 wt % to 5.0 wt %, preferably from 0.05 wt % to 3.0 wt %, even more preferably from 0.1 wt % to 1.0 wt % of at least one skin conditioning agent; and/or optionally comprises from 0.001 wt % to 10 wt %, preferably from 0.05 wt % to 5.0 wt %, even more preferably from 0.1 wt % to 3.0 wt %, most preferably from 0.05 wt % to 1.0 wt % of at least one antioxidant; and/or comprises from 0.001 wt % to 5.0 wt %, preferably from 0.01 wt % to 3.0 wt %, even more preferably from 0.1 wt % to 2.0 wt % of at least one biogenic active substance; and/or optionally comprises from 0.01 wt % to 4.0 wt %, preferably from 0.1 wt % to 3.0 wt %, even more preferably from 0.4 wt % to 1.0 wt % of at least one preservative or preservation boosting ingredient; and/or optionally comprises from 0.01 wt % to 3.0 wt %, preferably from 0.05 wt % to 2.0 wt %, even more preferably from 0.1 wt % to 1.0 wt % of at least one perfume or fragrance ingredient; and/or optionally comprises from 0.01 wt % to 20 wt %, preferably from 0.1 wt % to 10 wt %, even more preferably from 0.5 wt % to 5.0 wt % of at least one further auxiliary.

Embodiment 86 provides the cosmetic composition of embodiments 85, wherein the composition has a pH value from 2.0 to 10.0, preferably from 3.0 to 9.0, even more preferably from 4.0 to 8.0 and very preferably from 5.0 to 7.0.

Embodiment 87 provides the cosmetic composition of any one of embodiments 34-83, wherein the composition is a body or face care composition such as body washes, face washes, micellar waters or gels, body scrubs, face peeling, facial exfoliators, liquid soaps, bath additives, bubble baths, shower creams or milks, shower foams and face masks; and for example, optionally comprises from 0.5 wt % to 25 wt %, preferably from 1.0 wt % to 20 wt %, even more preferably from 2.0 wt % to 15 wt % of at least one surfactant; and/or optionally comprises from 0.01 wt % to 15 wt %, preferably from 0.1 wt % to 10 wt %, even more preferably from 0.5 wt % to 5.0 wt % of at least one viscosity modifier or thickening agent; and/or optionally comprises from 0.01 wt % to 15 wt %, preferably from 0.05 wt % to 10 wt %, even more preferably from 0.5 wt % to 5.0 wt % of at least one particulate substance; and/or optionally comprises from 0.001 wt % to 30 wt %, preferably from 0.05 wt % to 20 wt %, even more preferably from 0.1 wt % to 10 wt % of at least one oily substance; and/or optionally comprises from 0.05 wt % to 15 wt %, preferably from 0.1 wt % to 10 wt %, even more preferably from 0.5 wt % to 5.0 wt % of at least one functional acid or/and an active ingredient; and/or optionally comprises from 0.001 wt % to 5.0 wt %, preferably from 0.05 wt % to 3.0 wt %, even more preferably from 0.1 wt % to 1.0 wt % of at least one electrolyte; and/or optionally comprises from 0.01 wt % to 5.0 wt %, preferably from 0.1 wt % to 3.0 wt %, even more preferably from 0.4 wt % to 1.0 wt % of at least one preservative or preservation boosting ingredient; and/or optionally comprises from 0.01 wt % to 20 wt %, preferably from 0.1 wt % to 10 wt %, even more preferably from 0.5 wt % to 5.0 wt % of at least one further auxiliary.

Embodiment 88 provides the cosmetic composition of embodiments 87, wherein the composition has a pH value from 2.0 to 10.0, preferably from 3.0 to 8.0, even more preferably from 4.0 to 7.0.

Embodiment 89 provides the cosmetic composition of any one of embodiments 34-83, wherein the composition is a sun care composition such as sun sprays, sun milks, sun lotions, sun gels; and for example, optionally comprises from 0.1 wt % to 15 wt %, preferably from 0.5 wt % to 10 wt %, even more preferably from 1.0 wt % to 5.0 wt % of at least one emulsifier, coemulsifier and/or solubilizer; and/or optionally comprises from 0.001 wt % to 50 wt %, preferably from 0.05 wt % to 40 wt %, even more preferably from 0.1 wt % to 30 wt % of at least one oily substance; and/or optionally comprises from 0.001 wt % to 30 wt %, preferably from 0.05 wt % to 20 wt %, even more preferably from 0.1 wt % to 10 wt %, most preferably from 0.05 wt % to 5.0 wt % of at least one sun protection agent and/or UV filter; and/or optionally comprises from 0.1 wt % to 10 wt %, preferably from 0.5 wt % to 7.5 wt %, even more preferably from 1.0 wt % to 5.0 wt % of at least one film former; and/or optionally comprises from 0.01 wt % to 5.0 wt %, preferably from 0.1 wt % to 3.0 wt %, even more preferably from 0.4 wt % to 1.0 wt % of at least one preservative or preservation boosting ingredient; and/or optionally comprises from 0.01 wt % to 20 wt %, preferably from 0.1 wt % to 10 wt %, even more preferably from 0.5 wt % to 5.0 wt % of at least one further auxiliary.

Embodiment 90 provides the cosmetic composition of embodiments 89, wherein the composition has a pH value from 3.0 to 9.0, preferably from 4.0 to 8.0, even more preferably from 5.0 to 7.0.

Embodiment 91 provides the cosmetic composition of any one of embodiments 34-83, wherein the composition is a face toner; and for example, optionally comprises from 0.001 wt % to 5.0 wt %, preferably from 0.05 wt % to 3.0 wt %, even more preferably from 0.1 wt % to 1.0 wt % of at least one skin conditioning agent; and/or optionally comprises from 0.01 wt % to 15 wt %, preferably from 0.1 wt % to 10 wt %, even more preferably from 0.5 wt % to 5.0 wt % of at least one viscosity modifier or thickening agent; and/or optionally comprises from 0.01 wt % to 5.0 wt %, preferably from 0.1 wt % to 3.0 wt %, even more preferably from 0.4 wt % to 1.0 wt % of at least one preservative or preservation boosting ingredient; and/or optionally comprises from 0.01 wt % to 20 wt %, preferably from 0.1 wt % to 10.0 wt %, even more preferably from 0.5 wt % to 5.0 wt % of at least one further auxiliary.

Embodiment 92 provides the cosmetic composition of embodiments 91, wherein the composition has a pH value from 3.0 to 9.0, preferably from 4.0 to 8.0, even more preferably from 5.0 to 7.0.

Embodiment 93 provides the cosmetic composition of any one of embodiments 34-83, wherein the composition is a bar soap or syndet composition; and for example, optionally comprises from 1.0 wt % to 50 wt %, preferably from 2.0 wt % to 30 wt %, even more preferably from 5.0 wt % to 20 wt % of at least one surfactant; and/or optionally comprises from 0.01 wt % to 20 wt %, preferably from 0.05 wt % to 15 wt %, even more preferably from 0.5 wt % to 10 wt % of at least one particulate substance; and/or optionally comprises from 0.001 wt % to 5.0 wt %, preferably from 0.05 wt % to 3.0 wt %, even more preferably from 0.1 wt % to 1.0 wt % of at least one electrolyte; and/or optionally comprises from 0.01 wt % to 20 wt %, preferably from 0.1 wt % to 10 wt %, even more preferably from 0.5 wt % to 5.0 wt % of at least one further auxiliary.

Embodiment 94 provides the cosmetic composition of embodiments 93, wherein the composition has a pH value from 3.0 to 13.0, preferably from 4.0 to 12.0, even more preferably from 5.0 to 10.0.

Embodiment 95 provides the cosmetic composition of any one of embodiments 34-83, wherein the composition is suitable for wet wipee; and for example, optionally comprises from 0.1 wt % to 10 wt %, preferably from 0.3 wt % to 5.0 wt %, even more preferably from 0.5 wt % to 3.0 wt % of at least one emulsifier, coemulsifier and/or solubilizer; and/or optionally comprises from 0.001 wt % to 30 wt %, preferably from 0.05 wt % to 20 wt %, even more preferably from 0.1 wt % to 10 wt % of at least one oily substance; and/or optionally comprises from 0.01 wt % to 5.0 wt %, preferably from 0.1 wt % to 3.0 wt %, even more preferably from 0.4 wt % to 1.0 wt % of at least one preservative or preservation boosting ingredient; and/or optionally comprises from 0.01 wt % to 20 wt %, preferably from 0.1 wt % to 10 wt %, even more preferably from 0.5 wt % to 5.0 wt % of at least one further auxiliary.

Embodiment 96 provides the cosmetic composition of embodiments 95, wherein the composition has a pH value from 2.0 to 10, preferably from 3.0 to 9.0, even more preferably from 4.0 to 8.0.

Embodiment 97 provides the cosmetic composition of any one of embodiments 34-83, wherein the composition is a deodorizing and/or antiperspirant composition, for example, in the form of a cream, a roll-on, a solid, an aerosol or a gel; and for example, optionally comprises from 0.001 wt % to 10 wt %, or from 0.01 wt % to 9.0 wt %, or from 0.05 wt % to 8.0 wt %, or from 0.1 wt % to 5.0 wt % of at least one antiperspirant and/or deodorizing agent; and/or optionally comprises 0.01 wt % to 3.0 wt %, preferably from 0.05 wt % to 2.0 wt %, even more preferably from 0.1 wt % to 1.0 wt % of at least one perfume or fragrance ingredient; and/or optionally comprises from 0.5 wt % to 60 wt %, preferably from 1.0 wt % to 50 wt %, even more preferably from 2.0 wt % to 40 wt % of at least one propellant; and/or optionally comprises from 0.01 wt % to 20 wt %, preferably from 0.1 wt % to 10 wt %, even more preferably from 0.5 wt % to 5.0 wt % of at least one further auxiliary.

Embodiment 98 provides the cosmetic composition of embodiments 97, wherein the composition has a pH value from 2.0 to 8.0, preferably from 3.0 to 7.0, even more preferably from 4.0 to 6.0.

Embodiment 99 provides the cosmetic composition of any one of embodiments 34-83, wherein the composition is a shaving composition; and for example, optionally comprises from 0.1 wt % to 10 wt %, preferably from 0.3 wt % to 5.0 wt %, even more preferably from 0.5 wt % to 3.0 wt % of at least one emulsifier, coemulsifier and/or solubilizer; and/or optionally comprises from 0.001 wt % to 5.0 wt %, preferably from 0.05 wt % to 3.0 wt %, even more preferably from 0.1 wt % to 1.0 wt % of at least one skin conditioning agent; and/or optionally comprises from 0.5 wt % to 60 wt %, preferably from 1.0 wt % to 50 wt %, even more preferably from 2.0 wt % to 40 wt % of at least one propellant; and/or optionally comprises from 0.1 wt % to 20 wt %, preferably from 0.3 wt % to 10 wt %, even more preferably from 0.5 wt % to 5.0 wt % of at least one surfactant; and/or optionally comprises from 0.01 wt % to 20 wt %, preferably from 0.1 wt % to 10 wt %, even more preferably from 0.5 wt % to 5.0 wt % of at least one further auxiliary.

Embodiment 100 provides the cosmetic composition of embodiments 99, wherein the composition has a pH value from 2.0 to 8.0, preferably from 3.0 to 7.0, even more preferably from 4.0 to 6.0.

Embodiment 101 provides the cosmetic composition of any one of embodiments 34-83, wherein the composition is for use on hair and/or scalp; e.g., the composition is an emulsion or gel, preferably an oil-in-water (o/w), cream gel, hydro-alcoholic gel or hydrogel composition.

Embodiment 102 provides the cosmetic composition of embodiments 101, wherein the hair care composition has a viscosity from 100 000 to 150 000 mPa·s, preferably from 1 000 to 100 000 mPa·s, more preferably from 2 000 to 50 000 mPa·s and very preferably from 5 000 to 30 000 mPa·s (25° C., Brookfield RVT, T-C spindle at 20 revolutions per minute).

Embodiment 103 provides the cosmetic composition of embodiments 101 or 102, wherein the hair care composition is a shampoo; and for example, optionally comprises from 0.5 wt % to 30 wt %, preferably from 1.0 wt % to 15 wt %, even more preferably from 2.0 wt % to 10 wt % of at least one surfactant; and/or optionally comprises from 0.1 wt % to 10 wt %, preferably from 0.5 wt % to 7.5 wt %, even more preferably from 1.0 wt % to 5.0 wt % of at least one cationic polymer; and/or optionally comprises from 0.1 wt % to 15 wt %, preferably from 0.5 wt % to 10 wt %, even more preferably from 1.0 wt % to 5.0 wt % of at least one pearlizing agent; and/or optionally comprises from 0.001 wt % to 5.0 wt %, preferably from 0.05 wt % to 3.0 wt %, even more preferably from 0.1 wt % to 1.0 wt % of at least one electrolyte; and/or optionally comprises from 0.01 wt % to 15 wt %, preferably from 0.1 wt % to 10 wt %, even more preferably from 0.5 wt % to 5.0 wt % of at least one viscosity modifier or thickening agent; and/or optionally comprises from 0.01 wt % to 5.0 wt %, preferably from 0.1 wt % to 3.0 wt %, even more preferably from 0.4 wt % to 1.0 wt % of at least one preservative or preservation boosting ingredient; and/or optionally comprises from 0.01 wt % to 20 wt %, preferably from 0.1 wt % to 10 wt %, even more preferably from 0.5 wt % to 5.0 wt % of at least one further auxiliary.

Embodiment 104 provides the cosmetic composition of embodiments 101 or 102, wherein the hair care composition is a hair conditioning and/or hair and/or scalp treatment composition such as leave-in and rinse-off conditioners, masks, lotions, combing creams, detangling creams, anti-frizz liquids, hair serums, scalp serums, color protection creams; and for example, optionally comprises from 0.1 wt % to 15 wt %, preferably from 0.5 wt % to 10 wt %, even more preferably from 1.0 wt % to 5.0 wt % of at least one emulsifier, coemulsifier and/or solubilizer; and/or optionally comprises from 0.01 wt % to 20 wt %, preferably from 0.05 wt % to 10 wt %, even more preferably from 0.1 wt % to 5.0 wt % of at least one oily substance; and/or optionally comprises from 0.1 wt % to 10 wt %, preferably from 0.5 wt % to 7.5 wt %, even more preferably from 1.0 wt % to 5.0 wt % of at least one cationic polymer; and/or optionally comprises from 0.001 wt % to 5.0 wt %, preferably from 0.01 wt % to 3.0 wt %, even more preferably from 0.1 wt % to 2.0 wt % of at least one biogenic active substance; and/or optionally comprises from 0.01 wt % to 5.0 wt %, preferably from 0.1 wt % to 3.0 wt %, even more preferably from 0.4 wt % to 1.0 wt % of at least one preservative or preservation boosting ingredient; and/or optionally comprises from 0.01 wt % to 20 wt %, preferably from 0.1 wt % to 10 wt %, even more preferably from 0.5 wt % to 5.0 wt % of at least one further auxiliary.

Embodiment 105 provides the cosmetic composition of embodiments 101 or 102, wherein the hair care composition is a hair styling composition such as mousses, gels, sprays and waxes; and for example, optionally comprises from 0.1 wt % to 10 wt %, preferably from 0.5 wt % to 7.5 wt %, even more preferably from 1.0 wt % to 5.0 wt % of at least one film former; and/or optionally comprises from 0.1 wt % to 10 wt %, preferably from 0.5 wt % to 7.5 wt %, even more preferably from 1.0 wt % to 5.0 wt % of at least one cationic polymer; and/or optionally comprises from 0.5 wt % to 60 wt %, preferably from 1.0 wt % to 50 wt %, even more preferably from 2.0 wt % to 40 wt % of at least one propellant; and/or optionally comprises from 0.01 wt % to 5.0 wt %, preferably from 0.1 wt % to 3.0 wt %, even more preferably from 0.4 wt % to 1.0 wt % of at least one preservative or preservation boosting ingredient; and/or optionally comprises from 0.01 wt % to 20 wt %, preferably from 0.1 wt % to 10.0 wt %, even more preferably from 0.5 wt % to 5.0 wt % of at least one further auxiliary.

Embodiment 106 provides the cosmetic composition of any one of embodiments 34-83, wherein the composition is a mouthwash composition; and for example, optionally comprises from 0.001 wt % to 3.0 wt %, preferably from 0.01 wt % to 2.0 wt %, even more preferably from 0.05 wt % to 1.0 wt % of at least one flavouring agent; and/or optionally comprises from 0.1 wt % to 6.0 wt %, preferably from 0.2 wt % to 4.0 wt %, even more preferably from 0.3 wt % to 2.0 wt % of at least one solubilizer; and/or optionally comprises from 0.001 wt % to 5.0 wt %, preferably from 0.05 wt % to 3.0 wt %, even more preferably from 0.1 wt % to 1.0 wt % of at least one electrolyte; and/or optionally comprises from 0.01 wt % to 5.0 wt %, preferably from 0.1 wt % to 3.0 wt %, even more preferably from 0.4 wt % to 1.0 wt % of at least one preservative or preservation boosting ingredient; and/or optionally comprises from 0.01 wt % to 20 wt %, preferably from 0.1 wt % to 10 wt %, even more preferably from 0.5 wt % to 5.0 wt % of at least one further auxiliary.

Embodiment 107 provides the cosmetic composition of embodiments 106, wherein the composition has a pH value from 2.0 to 10.0, preferably from 3.0 to 9.0, even more preferably from 4.0 to 8.0.

Embodiment 108 provides a method of caring for keratinous material, comprising:
(1) Applying the cosmetic composition according to any one of embodiments 34-107 or the antimicrobial combination composition according any one of embodiments 1-30 onto keratinous material; and
(2) Allowing the composition to remain on the keratinous material for at least 1 hour.

Embodiment 109 provides a method according to embodiment 108, wherein the method comprises applying the cosmetic composition according to any one of embodiments 34-107 onto keratinous material; and allowing the composition to remain on the keratinous material for at least 1 hour.

Embodiment 120 provides a method according to embodiment 108 or 109, wherein the method does not comprise a rinsing step (e.g., by refraining from rinsing the treated skin for at least 1 hour, at least 2 hours, at least 4 hours, or at least 6 hours).

Embodiment 121 provides use of the antimicrobial combination composition of any of embodiments 1-30, in preserving cosmetic compositions.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A cosmetic composition for leave-on skin care use, the cosmetic composition having a pH in the range of 4-6, the cosmetic composition comprising:
(a) one or more glycerol derivatives selected from the group consisting of: 3-[(2-ethylhexyl)oxy]-1,2-propanediol, 3-[(n-octyl)oxy]-1,2-propanediol, and 3-[(2-octyl)oxy]-1,2-propanediol; and
(b) one or more bicyclic compounds selected from the group consisting of: 5,7-dihydroxy-2-(4-hydroxyphenyl)chroman-4-one, 2-(3,4-dihydroxyphenyl)-3,5,7-trihydroxychromen-4-one, 5,7-dihydroxy-2-(3-hydroxy-4-methoxyphenyl)chroman-4-one, and 5,7-dihydroxy-2-(3,4-dihydroxyphenyl)-chroman-4-one.

2. The cosmetic composition according to claim 1, wherein the composition comprises less than 5 wt. % of the one or more of the bicyclic compounds, based on the total weight of the one or more glycerol derivatives.

3. The cosmetic composition according to claim 1, wherein the composition has a pH within the range of 5.3 to 5.7.

4. The cosmetic composition according to claim 1, wherein the one or more glycerol derivatives includes 3-[(2-ethyl hexyl)oxy]-1,2-propanediol.

5. The cosmetic composition according to claim 1, wherein the one or more glycerol derivatives includes 3-[(n-octyl)oxy]-1,2-propanediol.

6. The cosmetic composition according to claim 1, wherein the one or more glycerol derivatives includes 3-[(2-octyl)oxy]-1,2-propanediol.

7. The cosmetic composition according to claim 1, wherein the one or more bicyclic compounds includes 5,7-dihydroxy-2-(4-hydroxyphenyl)chroman-4-one.

8. The cosmetic composition according to claim 1, wherein the one or more bicyclic compounds includes 2-(3,4-dihydroxyphenyl)-3,5,7-trihydroxychromen-4-one.

9. The cosmetic composition according to claim 1, wherein the one or more bicyclic compounds includes 5,7-dihydroxy-2-(3-hydroxy-4-methoxyphenyl)chroman-4-one.

10. The cosmetic composition according to claim 1, wherein the one or more bicyclic compounds includes 5,7-dihydroxy-2-(3,4-dihydroxyphenyl)-chroman-4-one.

11. A method of caring for skin, comprising:
a. applying the cosmetic composition according to claim 1 onto skin; and
b. allowing the composition to remain on the skin for at least 1 hour.

12. The method according to claim 11, wherein the method does not comprise a rinsing step.

13. The method according to claim 11, wherein the method includes refraining from rinsing the treated skin for at least 2 hours.

14. The cosmetic composition according to claim 1, wherein the one or more bicyclic compounds is present in an amount in the range of about 50 ppm to about 50000 ppm, based on the total weight of the one or more glycerol derivatives present in the cosmetic composition.

15. The cosmetic composition according to claim 1, wherein the one or more glycerol derivatives is present in an amount in the range of about 0.0005 wt % to about 5 wt % based on the total weight of the cosmetic composition.

16. The cosmetic composition according to claim 1, further comprising one or more surfactants.
